(19) United States
(12) Patent Application Publication
(10) Pub. No.: US 2024/0392363 A1
(43) Pub. Date: Nov. 28, 2024

(54) METHODS FOR DISCRIMINATING BETWEEN FETAL AND MATERNAL EVENTS IN NON-INVASIVE PRENATAL TEST SAMPLES

(71) Applicants: Illumina, Inc., San Diego, CA (US); Verinata Health, Inc., Redwood City, CA (US)

(72) Inventors: Victoria COREY, Carlsbad, CA (US); Mike MEHAN, San Marcos, CA (US); Sung KIM, San Diego, CA (US)

(21) Appl. No.: 18/670,057

(22) Filed: May 21, 2024

Related U.S. Application Data
(60) Provisional application No. 63/469,152, filed on May 26, 2023.

(51) Int. Cl. C12Q 1/6869

(52) U.S. Cl. CPC C12Q 1/6869

(57) ABSTRACT

The technology relates in part to methods for determining copy number variations (CNVs) known or suspected to be associated with a variety of medical conditions. In some aspects, the technology relates to determining CNVs of fetuses using maternal samples comprising maternal and fetal cell free DNA. In some aspects, the technology relates to determining CNVs known or suspected to be associated with a variety of medical conditions. In some aspects, methods to improve the sensitivity and/or specificity of sequence data analysis by deriving a fragment size parameter are provided. In some aspects, information from fragments of different sizes is used to evaluate copy number variations. In some aspects, information from fragments of different sizes is used to discriminate between fetal and maternal CNVs. In some aspects, the technology relates to systems and computer program products for evaluation of CNVs of sequences of interest.

### RELATED PATENT APPLICATION(S)

This patent application claims the benefit of U.S. provisional patent application No. 63/469,152 filed on May 26, 2023, entitled METHODS FOR DISCRIMINATING BETWEEN FETAL AND MATERNAL EVENTS IN NON-INVASIVE PRENATAL TEST SAMPLES, naming Victoria Corey et al. as inventors, and designated by attorney docket no. ILM-1002PROV. The entire content of the foregoing patent application is incorporated herein by reference for all purposes, including all text, tables and drawings.

### FIELD

The technology relates in part to methods for determining copy number variations (CNVs) known or suspected to be associated with a variety of medical conditions. In some aspects, the technology relates to determining CNVs of fetuses using maternal samples comprising maternal and fetal cell free DNA. In some aspects, the technology relates to determining CNVs known or suspected to be associated with a variety of medical conditions. In some aspects, methods to improve the sensitivity and/or specificity of sequence data analysis by deriving a fragment size parameter are provided. In some aspects, information from fragments of different sizes is used to evaluate copy number variations. In some aspects, information from fragments of different sizes is used to discriminate between fetal and maternal CNVs. In some aspects, the technology relates to systems and computer program products for evaluation of CNVs of sequences of interest.

### BACKGROUND

One of the critical endeavors in human medical research is the discovery of genetic abnormalities that produce adverse health consequences. In many cases, specific genes and/or critical diagnostic markers have been identified in portions of the genome that are present at abnormal copy numbers. For example, in prenatal diagnosis, extra or missing copies of whole chromosomes are frequently occurring genetic lesions. In cancer, deletion or multiplication of copies of whole chromosomes or chromosomal segments, and higher-level amplifications of specific regions of the genome, are common occurrences.

Most information about copy number variations (CNVs) has been provided by cytogenetic resolution that has permitted recognition of structural abnormalities. Conventional procedures for genetic screening and biological dosimetry have utilized invasive procedures, e.g., amniocentesis, cordocentesis, or chorionic villus sampling (CVS), to obtain cells for the analysis of karyotypes. Recognizing the need for more rapid testing methods that do not require cell culture, fluorescence in situ hybridization (FISH), quantitative fluorescence PCR (QF-PCR) and array-Comparative Genomic Hybridization (array-CGH) have been developed as molecular-cytogenetic methods for the analysis of copy number variations.

The advent of technologies that allow for sequencing entire genomes in relatively short time, and the discovery of circulating cell-free DNA (cfDNA) have provided the opportunity to compare genetic material originating from one chromosome to be compared to that of another without the risks associated with invasive sampling methods, which provides a tool to diagnose various kinds of copy number variations of genetic sequences of interest.

Limitations of existing methods in noninvasive prenatal diagnostics, which include insufficient sensitivity stemming from the limited levels of cfDNA, and the sequencing bias of the technology stemming from the inherent nature of genomic information, underlie the continuing need for noninvasive methods that would provide any or all of the specificity, sensitivity, and applicability, to reliably diagnose copy number changes in a variety of clinical settings. It has been shown that the average lengths of the fetal cfDNA fragments are shorter than the maternal cfDNA fragments in the plasma of pregnant women. This difference between maternal and fetal cfDNA is exploited in the implementation herein to determine CNV and/or fetal fraction. Embodiments disclosed herein fulfill some of the above needs. Some embodiments provide high analytical sensitivity and specificity for noninvasive prenatal diagnostics and diagnoses of a variety of diseases.

### SUMMARY

Provided in certain aspects are methods for determining presence, absence, or no call of a copy number variation (CNV) of a nucleic acid sequence of interest in a test sample comprising cell-free nucleic acid fragments originating from two or more genomes, the method comprising: (a) receiving sequence reads obtained by sequencing the cell-free nucleic acid fragments in the test sample; (b) aligning the sequence reads of the cell-free nucleic acid fragments or aligning fragments containing the sequence reads to bins of a reference genome comprising the sequence of interest, thereby providing test sequence tags, where the reference genome is divided into a plurality of bins; (c) measuring fragment lengths of at least some of the cell-free nucleic acid fragments present in the test sample; (d) for the sequence of interest: (i) generating a first test sequence tag quantification for fragments within a first selected fragment length range; and (ii) generating a second test sequence tag quantification for fragments within a second selected fragment length range, where the first selected fragment length range and the second selected fragment length range are different; and (e) determining the presence, absence, or no call of a copy number variation in the sequence of interest according to the first test sequence tag quantification and the second test sequence tag quantification.

Also provided in certain aspects are systems comprising one or more microprocessors and memory, which memory comprises instructions executable by the one or more microprocessors and which memory comprises sequence reads obtained by sequencing cell-free nucleic acid fragments originating from two or more genomes in a test sample, and where the instructions executable by the one or more microprocessors are configured to: (a) align the sequence reads of the cell-free nucleic acid fragments or align fragments containing the sequence reads to bins of a reference genome comprising a sequence of interest, thereby providing test sequence tags, where the reference genome is divided into a plurality of bins; (b) measure fragment lengths of at least some of the cell-free nucleic acid fragments present in the test sample; (c) for the sequence of interest: (i) generate a first test sequence tag quantification for fragments within a first selected fragment length range; and (ii) generate a second test sequence tag quantification for fragments within a second selected fragment length range, where the first selected fragment length range and the second selected fragment length range are different; and (d) determine a presence, absence, or no call of a copy number variation in the sequence of interest according to the first test sequence tag quantification and the second test sequence tag quantification.

Also provided in certain aspects are machines comprising one or more microprocessors and memory, which memory comprises instructions executable by the one or more microprocessors and which memory comprises sequence reads obtained by sequencing cell-free nucleic acid fragments originating from two or more genomes in a test sample, and where the instructions executable by the one or more microprocessors are configured to: (a) align the sequence reads of the cell-free nucleic acid fragments or align fragments containing the sequence reads to bins of a reference genome comprising a sequence of interest, thereby providing test sequence tags, where the reference genome is divided into a plurality of bins; (b) measure fragment lengths of at least some of the cell-free nucleic acid fragments present in the test sample; (c) for the sequence of interest: (i) generate a first test sequence tag quantification for fragments within a first selected fragment length range; and (ii) generate a second test sequence tag quantification for fragments within a second selected fragment length range, where the first selected fragment length range and the second selected fragment length range are different; and (d) determine a presence, absence, or no call of a copy number variation in the sequence of interest according to the first test sequence tag quantification and the second test sequence tag quantification.

Also provided in certain aspects are non-transitory computer-readable storage media with an executable program stored thereon, where the program instructs a microprocessor to perform the following: (a) access sequence reads obtained by sequencing cell-free nucleic acid fragments originating from two or more genomes in a test sample; (b) align the sequence reads of the cell-free nucleic acid fragments or align fragments containing the sequence reads to bins of a reference genome comprising a sequence of interest, thereby providing test sequence tags, where the reference genome is divided into a plurality of bins; (c) measure fragment lengths of at least some of the cell-free nucleic acid fragments present in the test sample; (d) for the sequence of interest: (i) generate a first test sequence tag quantification for fragments within a first selected fragment length range; and (ii) generate a second test sequence tag quantification for fragments within a second selected fragment length range, where the first selected fragment length range and the second selected fragment length range are different; and (e) determine a presence, absence, or no call of a copy number variation in the sequence of interest according to the first test sequence tag quantification and the second test sequence tag quantification.

Certain implementations are described further in the following description, examples and claims, and in the drawings.

### INCORPORATION BY REFERENCE

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIGS. 1A-1C show fetal fraction vs. event signals for chromosome X in fetal XX samples.

FIGS. 2A-2C show fetal fraction vs. event signals for chromosome X in fetal XY samples.

FIGS. 3A-3C show short versus long read shifts in fetal samples.

FIG. 4 shows chromosome X shift in fetal females with maternal XXX.

### DETAILED DESCRIPTION

### Definitions

Unless otherwise indicated, the practice of the methods and systems disclosed herein involves techniques and apparatus commonly used in molecular biology, microbiology, protein purification, protein engineering, protein and DNA sequencing, and recombinant DNA fields, which are within the skill of the art. Such techniques and apparatus are known to those of skill in the art and are described in numerous texts and reference works (See e.g., Sambrook et al., “Molecular Cloning: A Laboratory Manual,” Third Edition (Cold Spring Harbor), [2001]); and Ausubel et al., “Current Protocols in Molecular Biology” [1987]).

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the specification as a whole. It is to be understood that this disclosure is not limited to the particular methodology, protocols, and reagents described, as these may vary, depending upon the context they are used by those of skill in the art. As used herein, the singular terms “a,” “an,” and “the” include the plural reference unless the context clearly indicates otherwise.

Unless otherwise indicated, nucleic acids are written left to right in 5′ to 3′ orientation and amino acid sequences are written left to right in amino to carboxy orientation, respectively.

The term “parameter” is used herein represents a physical feature whose value or other characteristic has an impact a relevant condition such as copy number variation. In some cases, the term parameter is used with reference to a variable that affects the output of a mathematical relation or model, which variable may be an independent variable (i.e., an input to the model) or an intermediate variable based on one or more independent variables. Depending on the scope of a model, an output of one model may become an input of another model, thereby becoming a parameter to the other model.

The term “fragment size parameter” refers to a parameter that relates to the size or length of a fragment or a collection of fragments such nucleic acid fragments; e.g., a cfDNA fragments obtained from a bodily fluid. A fragment size or size range may be a characteristic of a genome or a portion thereof when the genome produces nucleic acid fragments enriched in or having a higher concentration of the size or size range relative to nucleic acid fragments from another genome or another portion of the same genome.

The term “weighting” refers to modifying a quantity such as a parameter or variable using one or more values or functions, which are considered the “weight.” In certain embodiments, a parameter or variable is multiplied by the weight. In other embodiments, a parameter or variable is modified exponentially. In some embodiments, a function may be a linear or non-linear function. Examples of applicable non-linear functions include, but are not limited to Heaviside step functions, box-car functions, stair-case functions, or sigmoidal functions. Weighting an original parameter or variable may systematically increase or decrease the value of the weighted variable. In various embodiments, weighting may result in positive, non-negative, or negative values.

The term “copy number variation” (CNV) herein refers to variation in the number of copies of a nucleic acid sequence present in a test sample in comparison with the copy number of the nucleic acid sequence present in a reference sample. In certain embodiments, a nucleic acid sequence is 1 kb or larger. In some cases, a nucleic acid sequence is a whole chromosome or significant portion thereof. A “copy number variant” refers to a sequence of nucleic acid in which copy-number differences are found by comparison of a nucleic acid sequence of interest in test sample with an expected level of the nucleic acid sequence of interest. For example, the level of a nucleic acid sequence of interest in a test sample is compared to that present in a qualified sample. Copy number variants/variations include deletions, including microdeletions, insertions, including microinsertions, duplications, microduplications, multiplications, and translocations. CNVs encompass chromosomal aneuploidies and partial aneuploidies.

The term “aneuploidy” herein refers to an imbalance of genetic material caused by a loss or gain of a whole chromosome, or part of a chromosome.

The terms “chromosomal aneuploidy” and “complete chromosomal aneuploidy” herein refer to an imbalance of genetic material caused by a loss or gain of a whole chromosome, and includes germline aneuploidy and mosaic aneuploidy.

The terms “partial aneuploidy” and “partial chromosomal aneuploidy” herein refer to an imbalance of genetic material caused by a loss or gain of part of a chromosome, e.g., partial monosomy and partial trisomy, and encompasses imbalances resulting from translocations, deletions and insertions.

The term “plurality” refers to more than one element. For example, the term is used herein in reference to a number of nucleic acid molecules or sequence tags that are sufficient to identify significant differences in copy number variations in test samples and qualified samples using the methods disclosed herein. In some embodiments, at least about 3×10⁶ sequence tags of between about 20 and 40 bp are obtained for each test sample. In some embodiments, each test sample provides data for at least about 5×10⁶, 8×10⁶, 10×10⁶, 15×10⁶, 20×10⁶, 30×10⁶, 40×10⁶, or 50×10⁶ sequence tags, each sequence tag comprising between about 20 and 40 bp.

The term “paired end reads” refers to reads from paired end sequencing that obtains one read from each end of a nucleic acid fragment. Paired end sequencing may involve fragmenting strands of polynucleotides into short sequences called inserts. Fragmentation is optional or unnecessary for relatively short polynucleotides such as cell free DNA molecules.

The terms “polynucleotide,” “nucleic acid” and “nucleic acid molecules” are used interchangeably and refer to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The nucleotides include sequences of any form of nucleic acid, including, but not limited to RNA and DNA molecules such as cfDNA molecules. The term “polynucleotide” includes, without limitation, single- and double-stranded polynucleotide.

The term “test sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments, the sample comprises at least one nucleic acid sequence whose copy number is suspected of having undergone variation. Such samples include, but are not limited to sputum/oral fluid, amniotic fluid, blood, a blood fraction, or fine needle biopsy samples (e.g., surgical biopsy, fine needle biopsy, and the like), urine, peritoneal fluid, pleural fluid, and the like. Although the sample is often taken from a human subject (e.g., patient), the assays can be used to detect copy number variations (CNVs) in samples from any mammal, including, but not limited to dogs, cats, horses, goats, sheep, cattle, pigs, and the like. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, and the like. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “qualified sample” or “unaffected sample” herein refers to a sample comprising a mixture of nucleic acids that are present in a known copy number to which the nucleic acids in a test sample are to be compared, and it is a sample that is normal, i.e., not aneuploid, for the nucleic acid sequence of interest. In some embodiments, qualified samples are used as unaffected training samples of a training set to derive sequence profiles. In certain embodiments, qualified samples are used for identifying one or more normalizing chromosomes or segments for a chromosome under consideration. For example, qualified samples may be used for identifying a normalizing chromosome for chromosome 21. In such case, the qualified sample is a sample that is not a trisomy 21 sample. Another example involves using only females as qualifying samples for chromosome X. Qualified samples may also be employed for other purposes such as determining thresholds for calling affected samples, determining expected coverage quantities for different regions of a genome, and the like.

The term “training set” herein refers to a set of training samples that can comprise affected and/or unaffected samples and are used to develop a model for analyzing test samples. In some embodiments, the training set includes unaffected samples. In these embodiments, thresholds for determining CNVs are established using training sets of samples that are unaffected for the copy number variation of interest. The unaffected samples in a training set may be used as the qualified samples to identify normalizing sequences, e.g., normalizing chromosomes, and the chromosome doses of unaffected samples are used to set the thresholds for each of the sequences, e.g., chromosomes, of interest. In some embodiments, the training set includes affected samples. The affected samples in a training set can be used to verify that affected test samples can be easily differentiated from unaffected samples.

A training set is also a statistical sample in a population of interest, which statistical sample is not to be confused with a biological sample. A statistical sample often comprises multiple individuals, data of which individuals are used to determine one or more quantitative values of interest generalizable to the population. The statistical sample is a subset of individuals in the population of interest. The individuals may be persons, animals, tissues, cells, other biological samples (i.e., a statistical sample may include multiple biological samples), and other individual entities providing data points for statistical analysis.

Usually, a training set is used in conjunction with a validation set. The term “validation set” is used to refer to a set of individuals in a statistical sample, data of which individuals are used to validate or evaluate the quantitative values of interest determined using a training set.

“Evaluation of copy number” is used herein in reference to the statistical evaluation of the status of a genetic sequence related to the copy number of the sequence. For example, in some embodiments, the evaluation comprises the determination of the presence or absence of a genetic sequence. In some embodiments, the evaluation comprises the determination of the partial or complete aneuploidy of a genetic sequence. In other embodiments, the evaluation comprises discrimination between two or more samples based on the copy number of a genetic sequence. In some embodiments, the evaluation comprises statistical analyses, e.g., normalization and comparison, based on the copy number of the genetic sequence.

The term “qualified nucleic acid” is used interchangeably with “qualified sequence,” which is a sequence against which the amount of a sequence or nucleic acid of interest is compared. A qualified sequence is one present in a biological sample preferably at a known representation, i.e., the amount of a qualified sequence is known. Generally, a qualified sequence is the sequence present in a “qualified sample.” A “qualified sequence of interest” is a qualified sequence for which the amount is known in a qualified sample, and is a sequence that is associated with a difference of a sequence of interest between a control subject and an individual with a medical condition.

The term “sequence of interest” or “nucleic acid sequence of interest” herein refers to a nucleic acid sequence that is associated with a difference in sequence representation between healthy and diseased individuals. A sequence of interest can be a sequence on a chromosome that is misrepresented, i.e., over- or under-represented, in a disease or genetic condition. A sequence of interest may be a portion of a chromosome, i.e., chromosome segment, or a whole chromosome. For example, a sequence of interest can be a chromosome that is over-represented in an aneuploidy condition, or a gene encoding a tumor-suppressor that is under-represented in a cancer. A sequence of interest may comprise a chromosome or part thereof tested for presence, absence, or no call of a CNV. A sequence of interest may comprise a genomic or chromosome region tested for presence, absence, or no call of a microduplication or microdeletion. Sequences of interest include sequences that are over- or under-represented in the total population, or a subpopulation of cells of a subject. A “qualified sequence of interest” is a sequence of interest in a qualified sample. A “test sequence of interest” is a sequence of interest in a test sample.

The term “normalizing sequence” herein refers to a sequence that is used to normalize the number of sequence tags mapped to a sequence of interest associated with the normalizing sequence. In some embodiments, a normalizing sequence comprises a robust chromosome. A “robust chromosome” is one that is unlikely to be aneuploid. In some cases involving the human chromosome, a robust chromosome is any chromosome other than the X chromosome, Y chromosome, chromosome 13, chromosome 18, and chromosome 21. In some embodiments, the normalizing sequence displays a variability in the number of sequence tags that are mapped to it among samples and sequencing runs that approximates the variability of the sequence of interest for which it is used as a normalizing parameter. The normalizing sequence can differentiate an affected sample from one or more unaffected samples. In some implementations, the normalizing sequence best or effectively differentiates, when compared to other potential normalizing sequences such as other chromosomes, an affected sample from one or more unaffected samples. In some embodiments, the variability of the normalizing sequence is calculated as the variability in the chromosome dose for the sequence of interest across samples and sequencing runs. In some embodiments, normalizing sequences are identified in a set of unaffected samples.

A “normalizing chromosome,” “normalizing denominator chromosome,” or “normalizing chromosome sequence” is an example of a “normalizing sequence.” A “normalizing chromosome sequence” can be composed of a single chromosome or of a group of chromosomes. In some embodiments, a normalizing sequence comprises two or more robust chromosomes. In certain embodiments, the robust chromosomes are all autosomal chromosomes other than chromosomes, X, Y, 13, 18, and 21. A “normalizing segment” is another example of a “normalizing sequence.” A “normalizing segment sequence” can be composed of a single segment of a chromosome or it can be composed of two or more segments of the same or of different chromosomes. In certain embodiments, a normalizing sequence is intended to normalize for variability such as process-related, interchromosomal (intra-run), and inter-sequencing (inter-run) variability.

The term “differentiability” herein refers to a characteristic of a normalizing chromosome that enables one to distinguish one or more unaffected, i.e., normal, samples from one or more affected, i.e., aneuploid, samples. A normalizing chromosome displaying the greatest “differentiability” is a chromosome or group of chromosomes that provides the greatest statistical difference between the distribution of chromosome doses for a chromosome of interest in a set of qualified samples and the chromosome dose for the same chromosome of interest in the corresponding chromosome in the one or more affected samples.

The term “variability” herein refers to another characteristic of a normalizing chromosome that enables one to distinguish one or more unaffected, i.e., normal, samples from one or more affected, i.e., aneuploid, samples. The variability of a normalizing chromosome, which is measured in a set of qualified samples, refers to the variability in the number of sequence tags that are mapped to it that approximates the variability in the number of sequence tags that are mapped to a chromosome of interest for which it serves as a normalizing parameter.

The term “sequence tag density” herein refers to the number of sequence reads that are mapped to a reference genome sequence, e.g., the sequence tag density for chromosome 21 is the number of sequence reads generated by the sequencing method that are mapped to chromosome 21 of the reference genome.

The term “sequence tag density ratio” herein refers to the ratio of the number of sequence tags that are mapped to a chromosome of the reference genome, e.g., chromosome 21, to the length of the reference genome chromosome.

The term “sequence dose” herein refers to a parameter that relates the number of sequence tags or another parameter identified for a sequence of interest and the number of sequence tags or the other parameter identified for the normalizing sequence. In some cases, the sequence dose is the ratio of the sequence tag coverage or the other parameter for a sequence of interest to the sequence tag coverage or the other parameter for a normalizing sequence. In some cases, the sequence dose refers to a parameter that relates the sequence tag density of a sequence of interest to the sequence tag density of a normalizing sequence. A “test sequence dose” is a parameter that relates the sequence tag density or the other parameter of a sequence of interest, e.g., chromosome 21, to that of a normalizing sequence, e.g., chromosome 9, determined in a test sample. Similarly, a “qualified sequence dose” is a parameter that relates the sequence tag density or the other parameter of a sequence of interest to that of a normalizing sequence determined in a qualified sample.

The term “coverage” refers to the abundance of sequence tags mapped to a defined sequence. Coverage can be quantitatively indicated by sequence tag density (or count of sequence tags), sequence tag density ratio, normalized coverage amount, adjusted coverage values, and the like. In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run.

In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

The term “coverage quantity” refers to a modification of raw coverage and often represents the relative quantity of sequence tags (sometimes called counts) in a region of a genome such as a bin. A coverage quantity may be obtained by normalizing, adjusting and/or correcting the raw coverage or count for a region of the genome. For example, a normalized coverage quantity for a region may be obtained by dividing the sequence tag count mapped to the region by the total number sequence tags mapped to the entire genome. Normalized coverage quantity allows comparison of coverage of a bin across different samples, which may have different depths of sequencing. It differs from sequence dose in that the latter is typically obtained by dividing by the tag count mapped to a subset of the entire genome. The subset is one or more normalizing segments or chromosomes. Coverage quantities, whether or not normalized, may be corrected for global profile variation from region to region on the genome, G-C fraction variations, outliers in robust chromosomes, and the like.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “parameter” herein refers to a numerical value that characterizes a property of a system. Frequently, a parameter numerically characterizes a quantitative data set and/or a numerical relationship between quantitative data sets. For example, a ratio (or function of a ratio) between the number of sequence tags mapped to a chromosome and the length of the chromosome to which the tags are mapped, is a parameter.

The terms “threshold value” and “qualified threshold value” herein refer to any number that is used as a cutoff to characterize a sample such as a test sample containing a nucleic acid from an organism suspected of having a medical condition. The threshold may be compared to a parameter value to determine whether a sample giving rise to such parameter value suggests that the organism has the medical condition. In certain embodiments, a qualified threshold value is calculated using a qualifying data set and serves as a limit of diagnosis of a copy number variation, e.g., an aneuploidy, in an organism. If a threshold is exceeded by results obtained from methods disclosed herein, a subject can be diagnosed with a copy number variation, e.g., trisomy 21. Appropriate threshold values for the methods described herein can be identified by analyzing normalized values (e.g., chromosome doses, NCVs or NSVs) calculated for a training set of samples. Threshold values can be identified using qualified (i.e., unaffected) samples in a training set which comprises both qualified (i.e., unaffected) samples and affected samples. The samples in the training set known to have chromosomal aneuploidies (i.e., the affected samples) can be used to confirm that the chosen thresholds are useful in differentiating affected from unaffected samples in a test set. The choice of a threshold is dependent on the level of confidence that the user wishes to have to make the classification. In some embodiments, the training set used to identify appropriate threshold values comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, or more qualified samples. It may be advantageous to use larger sets of qualified samples to improve the diagnostic utility of the threshold values.

The term “bin” refers to a segment of a sequence or a segment of a genome. In some embodiments, bins are contiguous with one another within the genome or chromosome. Each bin may define a sequence of nucleotides in a reference genome. Sizes of the bin may be 1 kb, 100 kb, 1 Mb, or greater, depending on the analysis required by particular applications and sequence tag density. In addition to their positions within a reference sequence, bins may have other characteristics such as sample coverage and sequence structure characteristics such as G-C fraction.

The term “normalized value” herein refers to a numerical value that relates the number of sequence tags identified for the sequence (e.g., chromosome or chromosome segment) of interest to the number of sequence tags identified for a normalizing sequence (e.g., normalizing chromosome or normalizing chromosome segment). For example, a “normalized value” can be a chromosome dose as described elsewhere herein, or it can be an NCV, or it can be an NSV as described elsewhere herein.

The term “read” refers to a sequence obtained from a portion of a nucleic acid sample. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

The term “genomic read” is used in reference to a read of any segments in the entire genome of an individual.

The term “sequence tag” is herein used interchangeably with the term “mapped sequence tag” or mapped sequence read” to refer to a sequence read that has been specifically assigned, i.e., mapped, to a larger sequence, e.g., a reference genome, by alignment. Mapped sequence tags are uniquely mapped to a reference genome, i.e., they are assigned to a single location to the reference genome. Unless otherwise specified, tags that map to the same sequence on a reference sequence are counted once. Tags may be provided as data structures or other assemblages of data. In certain embodiments, a tag contains a read sequence and associated information for that read such as the location of the sequence in the genome, e.g., the position on a chromosome. In certain embodiments, the location is specified for a positive strand orientation. A tag may be defined to allow a limited amount of mismatch in aligning to a reference genome. In some embodiments, tags that can be mapped to more than one location on a reference genome, i.e., tags that do not map uniquely, may not be included in the analysis.

The term “non-redundant sequence tag” refers to sequence tags that do not map to the same site, which is counted for the purpose of determining normalized chromosome values (NCVs) in some embodiments. Sometimes multiple sequence reads are aligned to the same locations on a reference genome, yielding redundant or duplicated sequence tags. In some embodiments, duplicate sequence tags that map to the same position are omitted or counted as one “non-redundant sequence tag” for the purpose of determining NCVs. In some embodiments, non-redundant sequence tags aligned to non-excluded sites are counted to yield “non-excluded-site counts” (NES counts) for determining NCVs.

The term “site” refers to a unique position (i.e., chromosome ID, chromosome position and orientation) on a reference genome. In some embodiments, a site may provide a position for a residue, a sequence tag, or a segment on a sequence.

“Excluded sites” are sites found in regions of a reference genome that have been excluded for the purpose of counting sequence tags. In some embodiments, excluded sites are found in regions of chromosomes that contain repetitive sequences, e.g., centromeres and telomeres, and regions of chromosomes that are common to more than one chromosome, e.g., regions present on the Y-chromosome that are also present on the X chromosome.

“Non-excluded sites” (NESs) are sites that are not excluded in a reference genome for the purpose of counting sequence tags.

“Non-excluded-site counts” (NES counts) are the numbers of sequence tags that are mapped to NESs on a reference genome. In some embodiments, NES counts are the numbers of non-redundant sequence tags mapped to NESs. In some embodiments, coverage and related parameters such normalized coverage quantities, global profile removed coverage quantities, and chromosome dose are based on NES counts. In one example, a chromosome dose is calculated as the ratio of the NES count for a chromosome of interest to the count for a normalizing chromosome.

Normalized chromosome value (NCV) relates coverage of a test sample to coverages of a set of training/qualified samples. In some embodiments, NCV is based on chromosome dose. In some embodiments, NCV relates to the difference between the chromosome dose of a chromosome of interest in a test sample and the mean of the corresponding chromosome dose in a set of qualified samples as, and can be calculated as:

$${NCV}_{ij} = \frac{{x}_{ij} - {\hat{μ}}_{j}}{{\hat{σ}}_{j}} \qquad Equation 1$$

where \hat{μ}ⱼ and \hat{σ}ⱼ are the estimated mean and standard deviation, respectively, for the j-th chromosome dose in a set of qualified samples, and xᵢⱼ is the observed j-th chromosome ratio (dose) for test sample i.

In some embodiments, NCV can be calculated “on the fly” by relating the chromosome dose of a chromosome of interest in a test sample to the median of the corresponding chromosome dose in multiplexed samples sequenced on the same flow cells as:

$${NCV}_{ij} = \frac{{x}_{ij} - {M}_{j}}{{\hat{σ}}_{j}} \qquad Equation 2$$

where Mⱼ is the estimated median for the j-th chromosome dose in a set of multiplexed samples sequenced on the same flow cell; \hat{σ}ⱼ is the standard deviation for the j-th chromosome dose in one or more sets of multiplexed samples sequenced on one or more flow cells, and xᵢⱼ is the observed j-th chromosome dose for test sample i. In this embodiment, test sample i is one of the multiplexed samples sequenced on the same flow cell from which Mⱼ is determined.

For example, for chromosome of interest 21 in test sample A, which is sequenced as one of 64 multiplexed samples on one flow cell, the NCV for chromosome 21 in test sample A is calculated as the dose of chromosome 21 in sample A minus the median of the dose for chromosome 21 determined in the 64 multiplexed samples, divided by the standard deviation of the dose for chromosome 21 determined for the 64 multiplexed samples on flow cell 1, or of additional flow cells.

As used herein, the terms “aligned,” “alignment,” or “aligning” refer to the process of comparing a read or tag to a reference sequence and thereby determining whether the reference sequence contains the read sequence. If the reference sequence contains the read, the read may be mapped to the reference sequence or, in certain embodiments, to a particular location in the reference sequence. In some cases, alignment simply tells whether or not a read is a member of a particular reference sequence (i.e., whether the read is present or absent in the reference sequence). For example, the alignment of a read to the reference sequence for human chromosome 13 will tell whether the read is present in the reference sequence for chromosome 13. A tool that provides this information may be called a set membership tester. In some cases, an alignment additionally indicates a location in the reference sequence where the read or tag maps to. For example, if the reference sequence is the whole human genome sequence, an alignment may indicate that a read is present on chromosome 13, and may further indicate that the read is on a particular strand and/or site of chromosome 13.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. One example of an algorithm from aligning sequences is the Efficient Local Alignment of Nucleotide Data (ELAND) computer program distributed as part of the Illumina Genomics Analysis pipeline. Alternatively, a Bloom filter or similar set membership tester may be employed to align reads to reference genomes. See U.S. Patent Application No. 61/552,374 filed Oct. 27, 2011 which is incorporated herein by reference in its entirety. The matching of a sequence read in aligning can be a 100% sequence match or less than 100% (non-perfect match).

The term “mapping” used herein refers to specifically assigning a sequence read to a larger sequence, e.g., a reference genome, by alignment.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. A “genome” refers to the complete genetic information of an organism or virus, expressed in nucleic acid sequences.

In various embodiments, the reference sequence is significantly larger than the reads that are aligned to it. For example, it may be at least about 100 times larger, or at least about 1000 times larger, or at least about 10,000 times larger, or at least about 10⁵ times larger, or at least about 10⁶ times larger, or at least about 10⁷ times larger.

In one example, the reference sequence is that of a full-length human genome. Such sequences may be referred to as genomic reference sequences. In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), and the like, of any species.

In various embodiments, the reference sequence is a consensus sequence or other combination derived from multiple individuals. However, in certain applications, the reference sequence may be taken from a particular individual.

The term “clinically-relevant sequence” herein refers to a nucleic acid sequence that is known or is suspected to be associated or implicated with a genetic or disease condition. Determining the absence or presence of a clinically-relevant sequence can be useful in determining a diagnosis or confirming a diagnosis of a medical condition, or providing a prognosis for the development of a disease.

The term “derived” when used in the context of a nucleic acid or a mixture of nucleic acids, herein refers to the means whereby the nucleic acid(s) are obtained from the source from which they originate. For example, in one embodiment, a mixture of nucleic acids that is derived from two different genomes means that the nucleic acids, e.g., cfDNA, were naturally released by cells through naturally occurring processes such as necrosis or apoptosis. In another embodiment, a mixture of nucleic acids that is derived from two different genomes means that the nucleic acids were extracted from two different types of cells from a subject.

The term “based on” when used in the context of obtaining a specific quantitative value, herein refers to using another quantity as input to calculate the specific quantitative value as an output.

The term “patient sample” herein refers to a biological sample obtained from a patient, i.e., a recipient of medical attention, care or treatment. The patient sample can be any of the samples described herein. In certain embodiments, the patient sample is obtained by non-invasive procedures, e.g., peripheral blood sample or a stool sample. The methods described herein need not be limited to humans. Thus, various veterinary applications are contemplated in which case the patient sample may be a sample from a non-human mammal (e.g., a feline, a porcine, an equine, a bovine, and the like).

The term “mixed sample” or “heterogeneous sample” herein refers to a sample containing a mixture of nucleic acids, which are derived from different genomes or sources. For example, mixed heterogeneous nucleic acid can include, but is not limited to, (i) fetal derived and maternal derived nucleic acid, (ii) cancer and non-cancer nucleic acid, (iii) pathogen and host nucleic acid, and more generally, (iv) mutated and wild-type nucleic acid or (v) majority and minority nucleic acid species. A sample may be heterogeneous because more than one cell type is present, such as a fetal cell and a maternal cell, a cancer and non-cancer cell, or a pathogenic and host cell. In some embodiments, a minority nucleic acid species and a majority nucleic acid species is present. Extracellular or cell-free nucleic acid can include different nucleic acid species, and therefore may be referred to herein as “heterogeneous” in certain embodiments.

The term “maternal sample” herein refers to a biological sample obtained from a pregnant subject, e.g., a woman.

The term “biological fluid” herein refers to a liquid taken from a biological source and includes, for example, blood, serum, plasma, sputum, lavage fluid, cerebrospinal fluid, urine, semen, sweat, tears, saliva, and the like. As used herein, the terms “blood,” “plasma” and “serum” expressly encompass fractions or processed portions thereof. Similarly, where a sample is taken from a biopsy, swab, smear, and the like, the “sample” expressly encompasses a processed fraction or portion derived from the biopsy, swab, smear, and the like.

The terms “maternal nucleic acids” and “fetal nucleic acids” herein refer to the nucleic acids of a pregnant female subject and the nucleic acids of the fetus being carried by the pregnant female, respectively.

As used herein, the term “corresponding to” sometimes refers to a nucleic acid sequence, e.g., a gene or a chromosome, that is present in the genome of different subjects, and which does not necessarily have the same sequence in all genomes, but serves to provide the identity rather than the genetic information of a sequence of interest, e.g., a gene or chromosome.

As used herein, the term “fetal fraction” refers to the fraction of fetal nucleic acids present in a sample comprising fetal and maternal nucleic acid. Fetal fraction is often used to characterize the cfDNA in a mother's blood.

As used herein, the term “tumor fraction” “cancer fraction” refers to the fraction of tumor nucleic acids present in a sample comprising tumor and host nucleic acid. Tumor fraction may be used to characterize circulating tumor DNA (ctDNA) in a patient's blood.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

As used herein, the term “polynucleotide length” refers to the absolute number of nucleotides in a sequence or in a region of a reference genome. The term “chromosome length” refers to the known length of the chromosome given in base pairs, e.g., provided in the NCBI36/hg18 assembly of the human chromosome found at |genome|.|ucsc|.|edu/cgi-bin/hgTracks?hgsid=167155613&chromInfoPage=on the World Wide Web.

The term “subject” herein refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

The term “condition” herein refers to “medical condition” as a broad term that includes all diseases and disorders, but can include injuries and normal health situations, such as pregnancy, that might affect a person's health, benefit from medical assistance, or have implications for medical treatments.

The term “complete” when used in reference to a chromosomal aneuploidy herein refers to a gain or loss of an entire chromosome.

The term “partial” when used in reference to a chromosomal aneuploidy herein refers to a gain or loss of a portion, i.e., segment, of a chromosome.

The term “mosaic” herein refers to denote the presence of two populations of cells with different karyotypes in one individual who has developed from a single fertilized egg. Mosaicism may result from a mutation during development which is propagated to only a subset of the adult cells.

The term “non-mosaic” herein refers to an organism, e.g., a human fetus, composed of cells of one karyotype.

The term “sensitivity” as used herein refers to the probability that a test result will be positive when the condition of interest is present. It may be calculated as the number of true positives divided by the sum of true positives and false negatives.

The term “specificity” as used herein refers to the probability that a test result will be negative when the condition of interest is absent. It may be calculated as the number of true negatives divided by the sum of true negatives and false positives.

The term “enrich” herein refers to the process of amplifying polymorphic target nucleic acids contained in a portion of a maternal sample, and combining the amplified product with the remainder of the maternal sample from which the portion was removed. For example, the remainder of the maternal sample can be the original maternal sample.

The term “original maternal sample” herein refers to a non-enriched biological sample obtained from a pregnant subject, e.g., a woman, who serves as the source from which a portion is removed to amplify polymorphic target nucleic acids. The “original sample” can be any sample obtained from a pregnant subject, and the processed fractions thereof, e.g., a purified cfDNA sample extracted from a maternal plasma sample.

The term “primer,” as used herein refers to an isolated oligonucleotide that is capable of acting as a point of initiation of synthesis when placed under conditions inductive to synthesis of an extension product (e.g., the conditions include nucleotides, an inducing agent such as DNA polymerase, and a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design.

### Introduction and Context

Copy number variations (CNVs) in the human genome can significantly influence human diversity and predisposition to diseases. Such diseases include, but are not limited to cancer, infectious and autoimmune diseases, diseases of the nervous system, metabolic and/or cardiovascular diseases, and the like.

CNVs may contribute to genetic disease through different mechanisms, resulting in either imbalance of gene dosage or gene disruption in most cases. In addition to their direct correlation with genetic disorders, CNVs can mediate phenotypic changes that can be deleterious. In certain cases, an increased burden of rare or de novo CNVs are present in complex disorders such as Autism, ADHD, and schizophrenia as compared to normal controls, highlighting the potential pathogenicity of rare or unique CNVs. CNVs generally arise from genomic rearrangements, primarily owing to deletion, duplication, insertion, and unbalanced translocation events.

NIPT (non-invasive prenatal testing) based on NGS data has been successfully implemented to detect CNVs in a fetus. Current methodologies involve sequencing maternal samples using short reads (25 bp-36 bp), aligning to the genome, computing and normalizing sub-chromosomal coverage, and finally evaluating over-representation of target chromosomes (e.g., 13/18/21/X/Y) compared to the expected normalized coverage associated with a normal diploid genome. Thus, traditional NIPT assays and analysis rely on counts or coverage to evaluate the likelihood of fetal aneuploidy.

Since maternal plasma samples represent a mixture of maternal and fetal cfDNA, the success of any given NIPT method often depends on its sensitivity to detect copy number changes in low fetal fraction samples. For counting based methods, their sensitivity is typically determined by (a) sequencing depth and (b) ability of data normalization to reduce technical variance. cfDNA fragments of fetal origin generally are shorter, on average, than those of maternal origin. This disclosure provides analytical methodology for NIPT and other applications by deriving fragment size information from, e.g., paired-end reads, and using this information in an analysis pipeline. Improved analytical sensitivity and/or specificity provides the ability to apply NIPT methods at reduced coverage (e.g., reduced sequencing depth) which enables the use of the technology for lower-cost testing of average risk pregnancies. Additionally, certain test samples may include CNVs from the mother, which can interfere with accurately diagnosing a CNV in a fetus (e.g., sometimes resulting in a false positive diagnosis for a fetus). Methods provided herein can discriminate between fetal and maternal CNVs, resulting in greater diagnostic accuracy.

Methods, apparatus, and systems are disclosed herein for determining copy number and copy number variations (CNVs) (i.e., determining presence, absence, or no call of CNVs) of different sequences of interest in a test sample that comprises a mixture of nucleic acids derived from two or more different genomes, and which are known or are suspected to differ in the amount of one or more sequence of interest. A “no call” result generally refers to a CNV that cannot be determined as present or absent with a certain degree of confidence. Copy number variations determined by the methods and apparatus disclosed herein include gains or losses of entire chromosomes, alterations involving very large chromosomal segments that are microscopically visible, and an abundance of sub-microscopic copy number variation of DNA segments ranging from single nucleotide, to kilobases (kb), to megabases (Mb) in size.

In some embodiments, methods are provided for determining copy number variations (CNVs) of fetuses using maternal samples containing maternal and fetal cell free DNA. Some implementations use fragment length (or fragment size) of cfDNA to improve sensitivity and specificity for fetal aneuploidy detection from cfDNA in maternal plasma. In some embodiments, both fragment size and coverage are utilized to enhance fetal aneuploidy detection. In some embodiments, both fragment size and coverage are utilized to discriminate between fetal and maternal aneuploidies.

Some embodiments disclosed provide methods to derive parameters with high signal to noise ratio from cell free nucleic acid fragments, for determining various genetic conditions related to copy number and CNVs, with improved sensitivity, specificity, selectivity, and/or efficiency relative to conventional methods. The parameters may include, but are not limited to, coverage, fragment size weighted coverage, fraction or ratio of coverage in a defined range, allele inheritance, fetal fraction estimates obtained from coverage information, etc. The depicted process has been found particularly effective at improving the signal in samples having relatively low fractions of DNA from a genome under consideration (e.g., a genome of a fetus). An example of such sample is a maternal blood sample from an individual pregnant with one or more fetuses.

In some embodiments, high analytical sensitivities and specificities can be achieved with a simple library preparation using very low cfDNA input that does not require PCR amplification. A PCR free method simplifies the workflow, improves the turn-around time and eliminates biases that are inherent with PCR methods. In some embodiments, the detection of fetal aneuploidy from maternal plasma can be made more robust and efficient than conventional methods, requiring fewer unique cfDNA fragments. In combination, improved analytical sensitivity and specificity is achieved with a very fast turnaround time at a significantly lower number of cfDNA fragments. This potentially allows NIPT to be carried out at significantly lower costs to facilitate application in the general obstetric population.

In various implementations, PCR-free library preparation is possible with the disclosed methods. Some implementations eliminate inherent biases of PCR methods, reduced assay complexity, reduce required sequencing depth (2.5× lower), provide faster turnaround time, e.g., turn around in one day, enable in-process fetal fraction (FF) measurement, and facilitate discrimination between maternal and fetal/placental cfDNA using fragment size information.

CNV detection according to sequence read quantifications and fragment lengths Provided herein are methods for detecting copy number variation in a test sample. In particular, provided herein are methods for determining presence, absence, or no call of a copy number variation (CNV) of a nucleic acid sequence of interest in a test sample comprising cell-free nucleic acid fragments originating from two or more genomes. In some embodiments, two or more genomes comprise a maternal genome and a fetal genome. In such embodiments, a test sample may be from a pregnant subject (i.e., a pregnant subject bearing a fetus). In some embodiments, two or more genomes comprise a host genome and a tumor genome. In such embodiments, a test sample may be from a cancer patient or a subject suspected of having cancer.

In some embodiments, length is measured for one or more nucleic acid fragments in a test sample (e.g., one or more cell-free nucleic acid fragments; one or more circulating cell-free nucleic acid fragments). In some embodiments, a sequence-based fragment length measurement is used. For example, nucleic acid fragment length may be measured using a paired-end sequencing platform, which generates paired-end sequence reads. Such platforms involve sequencing of both ends of a nucleic acid fragment. Generally, the sequences corresponding to both ends of the nucleic acid fragment can be aligned or mapped to a reference genome (e.g., a reference human genome). In certain embodiments, both ends are sequenced at a read length that is sufficient to align or map, individually for each fragment end, to a reference genome. In certain embodiments, all or a portion of the sequence reads can be aligned or mapped to a reference genome without mismatch. In some embodiments, each read is aligned or mapped independently. In some embodiments, information from both sequence reads (i.e., from each end of a nucleic acid fragment) is factored in the alignment/mapping process. The length of a nucleic acid fragment can be measured, for example, by calculating the difference between genomic coordinates assigned to each aligned/mapped paired-end read. In other words, the length of a nucleic acid fragment can be measured (e.g., deduced or inferred) by aligning/mapping two or more reads derived from the nucleic acid fragment (e.g., a paired-end read) to a reference genome. For paired-end reads derived from a nucleic acid fragment, for example, reads can be aligned/mapped to a reference genome, the length of the genomic sequence between the aligned/mapped reads can be determined, and the total of the two read lengths and the length of the genomic sequence between the reads is equal to the length of the nucleic acid fragment. In some embodiments, the length of a nucleic acid fragment is measured directly from the length of a read derived from the fragment (e.g., single-end read).

In certain applications of a method described herein, nucleic acid fragment length can be measured using any method in the art suitable for determining nucleic acid fragment length, such as, for example, a mass sensitive process (e.g., mass spectrometry (e.g., matrix-assisted laser desorption ionization (MALDI) mass spectrometry and electrospray (ES) mass spectrometry), electrophoresis (e.g., capillary electrophoresis), microscopy (scanning tunneling microscopy, atomic force microscopy), and measuring length using a nanopore. In some applications, fragment length may be determined by measuring the length of a probe that hybridizes to the fragment.

Methods herein may comprise generating one or more sequence tag quantifications (e.g., test sequence tag quantifications) for a sequence of interest. A sequence tag quantification for a sequence of interest generally refers to a representative count, or derivative thereof, for a sequence of interest. In some embodiments, a method herein comprises, for a sequence of interest, generating a first test sequence tag quantification for fragments within a first selected fragment length range and generating a second test sequence tag quantification for fragments within a second selected fragment length range. Accordingly, methods described herein may comprise categorizing nucleic acid fragment lengths according to a first selected fragment length range and a second selected fragment length range. Generally, a first selected fragment length range and a second selected fragment length range are different. Fragments having lengths within a first selected fragment length range may be referred to herein as short fragments. Fragments having lengths within a second selected fragment length range may be referred to herein as long fragments or all fragments.

In some embodiments, a first selected fragment length range is about 1 base to about 140 bases and the second selected fragment length range includes all fragment lengths. In some embodiments, a first selected fragment length range is about 1 base to about 150 bases and the second selected fragment length range includes all fragment lengths. In some embodiments, a first selected fragment length range is about 1 base to about 160 bases and the second selected fragment length range includes all fragment lengths.

In some embodiments, a first selected fragment length range is about 1 base to about 140 bases and the second selected fragment length range is about 141 bases and above. In some embodiments, a first selected fragment length range is about 1 base to about 150 bases and the second selected fragment length range is about 151 bases and above. In some embodiments, a first selected fragment length range is about 1 base to about 160 bases and the second selected fragment length range is about 161 bases and above.

In some embodiments, the first selected fragment length range is about 60 bases to about 170 bases. In some embodiments, the first selected fragment length range is about 70 bases to about 160 bases. In some embodiments, the first selected fragment length range is about 75 bases to about 155 bases. In some embodiments, the first selected fragment length range is about 80 bases to about 150 bases. In some embodiments, the second selected fragment length range is about 131 bases to about 400 bases. In some embodiments, the second selected fragment length range is about 141 bases to about 350 bases. In some embodiments, the second selected fragment length range is about 146 bases to about 325 bases. In some embodiments, the second selected fragment length range is about 151 bases to about 300 bases. In some embodiments, the first selected fragment length range is about 80 bases to about 150 bases and the second selected fragment length range is about 151 bases to about 300 bases.

In some embodiments, generating a first test sequence tag quantification comprises determining a number of sequence tags aligning to one or more bins in a sequence of interest, where the sequence tags are from fragments within a first selected fragment length range. In some embodiments, generating a first test sequence tag quantification comprises determining a number of sequence tags aligning to each bin in a sequence of interest, where the sequence tags are from fragments within a first selected fragment length range. In some embodiments, generating a second test sequence tag quantification comprises determining a number of sequence tags aligning to one or more bins in a sequence of interest, where the sequence tags are from fragments within a second selected fragment length range. In some embodiments, generating a second test sequence tag quantification comprises determining a number of sequence tags aligning to each bin in a sequence of interest, where the sequence tags are from fragments within a second selected fragment length range. The term “each bin” may refer to each and every bin in a sequence of interest, or may refer to each of certain or select bins in a sequence of interest.

In some embodiments, a number of sequence tags aligning to one or more bins in a sequence of interest is further processed. For example, generating a first and/or second test sequence tag quantification may comprise determining a measure of central tendency for the numbers of sequence tags aligning to the bins in the sequence of interest. A measure of central tendency may include a mean, average, median, or mode, for example. In some embodiments, a measure of central tendency is a mean. Accordingly, in some embodiments, a mean bin count for a sequence of interest is determined.

In some embodiments, a number of sequence tags aligning to one or more bins in a sequence of interest is normalized. Normalization may account for bin-to-bin variations. Typically, normalization accounts for bin-to-bin variations due to factors other than copy number variation. Any suitable normalization process described herein or known in the art may be used to normalize a number of sequence tags. In some embodiments, generating a first and/or second test sequence tag quantification further comprises normalizing a number of sequence tags aligning to each bin, thereby generating a normalized number of sequence tags aligning to each bin.

In some embodiments, a normalized number of sequence tags aligning to one or more bins in a sequence of interest is further processed. For example, generating a first and/or second test sequence tag quantification may comprise determining a measure of central tendency for the normalized numbers of sequence tags aligning to the bins in the sequence of interest. As noted above, a measure of central tendency may include a mean, average, median, or mode, for example. In some embodiments, a measure of central tendency is a mean. Accordingly, in some embodiments, a mean of normalized bin counts for a sequence of interest is determined.

In some embodiments, a first and/or second test sequence tag quantification comprises a shift from a fixed value or constant. A shift may be expressed as a subtraction of a bin count, a normalized bin count, a mean bin count, a mean of normalized bin counts, or the like, from a fixed value. A fixed value may be any suitable value (e.g., 0, 1, 2, 3, 4, 5 . . . etc.). In some embodiments, a fixed value is 1. In some embodiments, an absolute value of a shift from a fixed value is determined. Accordingly, in some embodiments, a first and/or second test sequence tag quantification comprises an absolute value of a shift from a fixed value.

In some embodiments, a first test sequence tag quantification is determined according to the following:

$$\vert 1 - {μ}_{first} \vert \qquad Equation 3$$

where μ_{first} is the mean of normalized numbers of sequence tags aligning to bins in a sequence of interest, where the sequence tags are from fragments within a first selected fragment length range.

In some embodiments, a second test sequence tag quantification is determined according to the following:

$$\vert 1 - {μ}_{second} \vert \qquad Equation 4$$

where μ_{second} is the mean of normalized numbers of sequence tags aligning to the bins in a sequence of interest, where the sequence tags are from fragments within a second selected fragment length range.

In some embodiments, a method herein comprises generating a ratio and determining a ratio value. In some embodiments, a method herein comprises generating a ratio and determining a ratio value for test sequence tag quantifications for a sequence of interest. In particular, methods herein may comprise generating a ratio of a first test sequence tag quantification to a second test sequence tag quantification (i.e., for a sequence of interest) and determining a ratio value. Accordingly, in some embodiments, a first test sequence tag quantification and a second test sequence tag quantification are generated and then a ratio of the first test sequence tag quantification and the second test sequence tag quantification is generated. Methods herein thus typically comprise generating a ratio of test sequence tag quantifications at the level of a sequence of interest and not at the individual bin level. In some embodiments, a method herein includes the following steps in sequential order: 1) obtain a first set of bin counts for bins in a sequence of interest according to sequence reads from fragments within a first selected fragment length range and obtain a second set of bin counts for bins in a sequence of interest according to sequence reads from fragments within a second selected fragment length range, 2) optionally normalize the bin counts for each bin for each set, 3) obtain the mean (or other measure of central tendency) of the bin counts or normalized bin counts for the sequence of interest for each set, and 4) generate a ratio of the mean bin counts, mean of the normalized bin counts, or a derivative thereof (e.g., shift from a fixed value, absolute value of a shift from a fixed value) for the first and second sets.

In some embodiments, a ratio value is determined according to the following:

$$ratio value = \frac{\vert 1 - {μ}_{first} \vert}{\vert 1 - {μ}_{second} \vert}$$

where μ_{first} is the mean of normalized numbers of sequence tags aligning to bins in a sequence of interest, where the sequence tags are from fragments within a first selected fragment length range; and μ_{second} is the mean of normalized numbers of sequence tags aligning to bins in a sequence of interest, where the sequence tags are from fragments within a second selected fragment length range.

Methods herein may comprise determining the presence, absence, or no call of a copy number variation in a sequence of interest. In particular, methods may comprise determining the presence, absence, or no call of a copy number variation in a sequence of interest according to a first test sequence tag quantification and a second test sequence tag quantification. In some embodiments, determining the presence, absence, or no call of a copy number variation in a sequence of interest is according to a ratio value of a first test sequence tag quantification to a second test sequence tag quantification.

In some embodiments, the presence, absence, or no call of a copy number variation is determined according to a decision boundary. A decision boundary may separate affected and unaffected samples above/below a boundary, left/right of a boundary, or inside/outside of a boundary. A decision boundary may be linear or non-linear. In some embodiments, the presence, absence, or no call of a copy number variation is determined according to a ratio value threshold. In some embodiments, a method herein comprises determining the presence of a copy number variation based, at least in part, on a determination that, for a test sample, the ratio value is greater than or equal to a ratio value threshold. In some embodiments, a method herein comprises determining the absence or no call of a copy number variation based, at least in part, on a determination that, for a test sample, the ratio value is less than a ratio value threshold.

A ratio value threshold may be determined, in certain instances, according to a training set or control set of samples with a known presence or absence of a CNV. A threshold that best separates affected samples from non-affected samples may be chosen. For example, a ratio value threshold may be any suitable value above 1 and up to 2 or more. In some embodiments, a ratio value threshold is about 1.1, 1.2, 1.3, 1.4, 1.5, 1.6, 1.7, 1.8, 1.9, or 2. In some embodiments, a ratio value threshold is between 1.1 and 1.3. In some embodiments, a ratio value threshold is 1.2.

In some embodiments, a method herein comprises determining a fraction of fragments originating from a first genome in the two or more genomes for a test sample. In some embodiments, a fraction of fragments originating from a first genome is a fetal fraction. In some embodiments, a fraction of fragments originating from a first genome is a tumor fraction. In some embodiments, a fraction of fragments originating from a first genome is determined according to a first test sequence tag quantification for fragments within a first selected fragment length range. In some embodiments, a fraction of fragments originating from a first genome is determined according to a normalized first test sequence tag quantification for fragments within a first selected fragment length range. In some embodiments, a fraction of fragments originating from a first genome corresponds to a shift of a first test sequence tag quantification from a fixed value. In some embodiments, a fraction of fragments originating from a first genome corresponds to an absolute value of a shift of a first test sequence tag quantification from a fixed value. In some embodiments, a fraction of fragments originating from a first genome corresponds to a shift of a normalized first test sequence tag quantification from a fixed value. In some embodiments, a fraction of fragments originating from a first genome corresponds to an absolute value of a shift of a normalized first test sequence tag quantification from a fixed value. A fixed value may be any suitable value (e.g., 0, 1, 2, 3, 4, 5 . . . etc.). In some embodiments, a fixed value is 1. In some embodiments, a first sequence tag quantification comprises of measure of central tendency. In some embodiments, a measure of central tendency is a mean. In some embodiments, a fraction of fragments originating from a first genome is determined according to a model (e.g., a liner model) as described herein and/or one or more model parameters as described herein (e.g., a model built from a training set of samples with known fragment fractions and measured sequence tag quantifications).

In some embodiments, a method herein comprises determining whether one or both genomes in the two or more genomes for a test sample comprise a CNV. For example, when the first genome comprises fetal DNA and the second genome comprises maternal DNA, a method herein comprises determining whether the fetus and the mother both carry the CNV. A determination that both genomes comprise the same CNV, may be based, in part, on a first test sequence tag quantification and a second test sequence tag quantification. When a first test sequence tag quantification (i.e., for fragments within a first selected fragment length range) is substantially equal to a second test sequence tag quantification (i.e., for fragments within a second selected fragment length range), it may be determined that the both genomes comprise the CNV (e.g., both the fetus and the mother carry the CNV). In some embodiments, when a first normalized test sequence tag quantification (i.e., for fragments within a first selected fragment length range) is substantially equal to a second normalized test sequence tag quantification (i.e., for fragments within a second selected fragment length range), it may be determined that the both genomes comprise the CNV (e.g., both the fetus and the mother carry the CNV). In some embodiments, when a shift of a first test sequence tag quantification (i.e., for fragments within a first selected fragment length range) from a fixed value is substantially equal to a shift of a second test sequence tag quantification (i.e., for fragments within a second selected fragment length range) from a fixed value, it may be determined that the both genomes comprise the CNV (e.g., both the fetus and the mother carry the CNV). In some embodiments, when a shift of a first normalized test sequence tag quantification (i.e., for fragments within a first selected fragment length range) from a fixed value is substantially equal to a shift of a second normalized test sequence tag quantification (i.e., for fragments within a second selected fragment length range) from a fixed value, it may be determined that the both genomes comprise the CNV (e.g., both the fetus and the mother carry the CNV). In some embodiments, when an absolute value of a shift of a first test sequence tag quantification (i.e., for fragments within a first selected fragment length range) from a fixed value is substantially equal to an absolute value of a shift of a second test sequence tag quantification (i.e., for fragments within a second selected fragment length range) from a fixed value, it may be determined that the both genomes comprise the CNV (e.g., both the fetus and the mother carry the CNV). In some embodiments, when an absolute value of a shift of a first normalized test sequence tag quantification (i.e., for fragments within a first selected fragment length range) from a fixed value is substantially equal to an absolute value of a shift of a second normalized test sequence tag quantification (i.e., for fragments within a second selected fragment length range) from a fixed value, it may be determined that the both genomes comprise the CNV (e.g., both the fetus and the mother carry the CNV). A fixed value may be any suitable value (e.g., 0, 1, 2, 3, 4, 5 . . . etc.). In some embodiments, a fixed value is 1. In some embodiments, a first sequence tag quantification and/or a second sequence tag quantification comprise of measure of central tendency. In some embodiments, a measure of central tendency is a mean.

In some embodiments, a determination that both genomes comprise the CNV, is based, in part, on a ratio of a first test sequence tag quantification (i.e., for fragments within a first selected fragment length range) to a second test sequence tag quantification (i.e., for fragments within a second selected fragment length range). In some embodiments, a determination that both genomes comprise the CNV, is based, in part, on a ratio of a first normalized test sequence tag quantification to a second normalized test sequence tag quantification. In some embodiments, a determination that both genomes comprise the CNV, is based, in part, on a ratio of a shift of a first test sequence tag quantification from a fixed value to a shift of a second test sequence tag quantification from a fixed value. In some embodiments, a determination that both genomes comprise the CNV, is based, in part, on a ratio of a shift of a first normalized test sequence tag quantification from a fixed value to a shift of a second normalized test sequence tag quantification from a fixed value. In some embodiments, a determination that both genomes comprise the CNV, is based, in part, on a ratio of an absolute value of a shift of a first test sequence tag quantification from a fixed value to an absolute value of a shift of a second test sequence tag quantification from a fixed value. In some embodiments, a determination that both genomes comprise the CNV, is based, in part, on a ratio of an absolute value of a shift of a first normalized test sequence tag quantification from a fixed value to an absolute value of a shift of a second normalized test sequence tag quantification from a fixed value. A fixed value may be any suitable value (e.g., 0, 1, 2, 3, 4, 5 . . . etc.). In some embodiments, a fixed value is 1. In some embodiments, a first sequence tag quantification comprises of measure of central tendency. In some embodiments, a measure of central tendency is a mean. In some embodiments, a determination that both genomes comprise the CNV, is based, in part, on a ratio value (e.g., determined from a ratio described above). In some embodiments, the ratio value is about 1.

In some embodiments, a method herein comprises determining presence or absence of a mosaicism for a genome in the two or more genomes for a test sample. In some embodiments, a method herein comprises determining a level of a mosaicism for a genome in the two or more genomes for a test sample. In some embodiments, the mosaicism is a maternal mosaicism. In some embodiments, the presence, absence, and/or level of maternal mosaicism is determined, in part, according to a second test sequence tag quantification (i.e., for fragments within a second selected fragment length range). In some embodiments, the presence, absence, and/or level of maternal mosaicism is determined, in part, according to a second normalized test sequence tag quantification (i.e., for fragments within a second selected fragment length range). In some embodiments, the presence, absence, and/or level of maternal mosaicism is determined, in part, according to a shift of a second test sequence tag quantification (i.e., for fragments within a second selected fragment length range) from a fixed value. In some embodiments, the presence, absence, and/or level of maternal mosaicism is determined, in part, according to a shift of a second normalized test sequence tag quantification (i.e., for fragments within a second selected fragment length range) from a fixed value. In some embodiments, the presence, absence, and/or level of maternal mosaicism is determined, in part, according to an absolute value of a shift of a second test sequence tag quantification (i.e., for fragments within a second selected fragment length range) from a fixed value. In some embodiments, the presence, absence, and/or level of maternal mosaicism is determined, in part, according to an absolute value of a shift of a normalized second test sequence tag quantification (i.e., for fragments within a second selected fragment length range) from a fixed value. A fixed value may be any suitable value (e.g., 0, 1, 2, 3, 4, 5 . . . etc.). In some embodiments, a fixed value is 1. In some embodiments, a second sequence tag quantification comprises of measure of central tendency. In some embodiments, a measure of central tendency is a mean. In some embodiments, a maternal mosaicism is detected when a test sequence tag quantification, or derivative thereof (e.g., normalized quantification, mean quantification, mean normalized quantification, shift from a fixed value, and/or absolute value thereof), deviates from a test sequence tag quantification from a sample having no maternal mosaicism. In some embodiments, a level of a maternal mosaicism is determined when a test sequence tag quantification, or derivative thereof (e.g., normalized quantification, mean quantification, mean normalized quantification, shift from a fixed value, and/or absolute value thereof), according to a level of deviation from a test sequence tag quantification from a sample having no maternal mosaicism. In some embodiments, the presence, absence, and/or level of maternal mosaicism is determined, in part, according to a model (e.g., a liner model) as described herein and/or one or more model parameters as described herein (e.g., a model built from a training set of samples with known mosaicisms and measured sequence tag quantifications).

### Nucleic Acid

Methods herein are generally directed to the analysis of nucleic acid. The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain instances. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. The term “gene” refers to the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons). Deoxyribonucleotides include deoxyadenosine, deoxycytidine, deoxyguanosine and deoxythymidine. For RNA, the base thymine is replaced with uracil. A template nucleic acid may be prepared using a nucleic acid obtained from a subject as a template.

Nucleic acids can include extracellular nucleic acid in certain embodiments. The term “extracellular nucleic acid” as used herein can refer to nucleic acid isolated from a source having substantially no cells and also is referred to as “cell-free” nucleic acid, “circulating cell-free nucleic acid” (e.g., CCF fragments) and/or “cell-free circulating nucleic acid”. Extracellular nucleic acid can be present in and obtained from blood (e.g., from the blood of a pregnant subject). Extracellular nucleic acid often includes no detectable cells and may contain cellular elements or cellular remnants. Non-limiting examples of acellular sources for extracellular nucleic acid are blood, blood plasma, blood serum, and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species, and therefore is referred to herein as “heterogeneous” in certain embodiments. For example, blood serum or plasma from a person having cancer can include nucleic acid from cancer cells and nucleic acid from non-cancer cells. In another example, blood serum or plasma from a pregnant subject can include maternal nucleic acid and fetal nucleic acid. In some instances, fetal nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is fetal nucleic acid). In some embodiments, the majority of fetal nucleic acid in nucleic acid is of a length of about 500 base pairs or less (e.g., about 80, 85, 90, 91, 92, 93, 94, 95, 96, 97, 98, 99 or 100% of fetal nucleic acid is of a length of about 500 base pairs or less). In some embodiments, the majority of fetal nucleic acid in nucleic acid is of a length of about 250 base pairs or less (e.g., about 80, 85, 90, 91, 92, 93, 94, 95, 96, 97, 98, 99 or 100% of fetal nucleic acid is of a length of about 250 base pairs or less). In some embodiments, the majority of fetal nucleic acid in nucleic acid is of a length of about 200 base pairs or less (e.g., about 80, 85, 90, 91, 92, 93, 94, 95, 96, 97, 98, 99 or 100% of fetal nucleic acid is of a length of about 200 base pairs or less). In some embodiments, the majority of fetal nucleic acid in nucleic acid is of a length of about 150 base pairs or less (e.g., about 80, 85, 90, 91, 92, 93, 94, 95, 96, 97, 98, 99 or 100% of fetal nucleic acid is of a length of about 150 base pairs or less). In some embodiments, the majority of fetal nucleic acid in nucleic acid is of a length of about 100 base pairs or less (e.g., about 80, 85, 90, 91, 92, 93, 94, 95, 96, 97, 98, 99 or 100% of fetal nucleic acid is of a length of about 100 base pairs or less). In some embodiments, the majority of fetal nucleic acid in nucleic acid is of a length of about 50 base pairs or less (e.g., about 80, 85, 90, 91, 92, 93, 94, 95, 96, 97, 98, 99 or 100% of fetal nucleic acid is of a length of about 50 base pairs or less). In some embodiments, the majority of fetal nucleic acid in nucleic acid is of a length of about 25 base pairs or less (e.g., about 80, 85, 90, 91, 92, 93, 94, 95, 96, 97, 98, 99 or 100% of fetal nucleic acid is of a length of about 25 base pairs or less).

Nucleic acid may be single or double stranded. Single stranded DNA, for example, can be generated by denaturing double stranded DNA by heating or by treatment with alkali, for example.

In certain embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

### Samples

Samples that are used for determining a CNV, e.g., chromosomal aneuploidy, partial aneuploidy, and the like, can include samples taken from any cell, tissue, or organ in which copy number variations for one or more sequences of interest are to be determined. Desirably, the samples contain nucleic acids that are that are present in cells and/or nucleic acids that are “cell-free” (e.g., cfDNA).

In some embodiments, it is advantageous to obtain cell-free nucleic acids, e.g., cell-free DNA (cfDNA). Cell-free nucleic acids, including cell-free DNA, can be obtained by various methods known in the art from biological samples including but not limited to plasma, serum, and urine (see, e.g., Fan et al., Proc Natl Acad Sci 105:16266-16271 [2008]; Koide et al., Prenatal Diagnosis 25:604-607 [2005]; Chen et al., Nature Med. 2: 1033-1035 [1996]; Lo et al., Lancet 350: 485-487 [1997]; Botezatu et al., Clin Chem. 46: 1078-1084, 2000; and Su et al., J Mol. Diagn. 6: 101-107 [2004]). To separate cell-free DNA from cells in a sample, various methods including, but not limited to fractionation, centrifugation (e.g., density gradient centrifugation), DNA-specific precipitation, or high-throughput cell sorting and/or other separation methods can be used. Commercially available kits for manual and automated separation of cfDNA are available (Roche Diagnostics, Indianapolis, IN; Qiagen, Valencia, CA; Macherey-Nagel, Duren, DE). Biological samples comprising cfDNA have been used in assays to determine the presence or absence of chromosomal abnormalities, e.g., trisomy 21, by sequencing assays that can detect chromosomal aneuploidies and/or various polymorphisms.

In various embodiments, the cfDNA present in the sample can be enriched specifically or non-specifically prior to use (e.g., prior to preparing a sequencing library). Non-specific enrichment of sample DNA refers to the whole genome amplification of the genomic DNA fragments of the sample that can be used to increase the level of the sample DNA prior to preparing a cfDNA sequencing library. Non-specific enrichment can be the selective enrichment of one of the two genomes present in a sample that comprises more than one genome. For example, non-specific enrichment can be selective of the fetal genome in a maternal sample, which can be obtained by suitable methods to increase the relative proportion of fetal to maternal DNA in a sample. Alternatively, non-specific enrichment can be the non-selective amplification of both genomes present in the sample. For example, non-specific amplification can be of fetal and maternal DNA in a sample comprising a mixture of DNA from the fetal and maternal genomes. Methods for whole genome amplification are known in the art. Degenerate oligonucleotide-primed PCR (DOP), primer extension PCR technique (PEP) and multiple displacement amplification (MDA) are examples of whole genome amplification methods. In some embodiments, a sample comprising a mixture of cfDNA from different genomes is un-enriched for cfDNA of the genomes present in the mixture. In other embodiments, a sample comprising a mixture of cfDNA from different genomes is non-specifically enriched for any one of the genomes present in the sample.

The sample comprising the nucleic acid(s) to which the methods described herein are applied typically comprises a biological sample (“test sample”), e.g., as described above. In some embodiments, the nucleic acid(s) to be screened for one or more CNVs is purified or isolated by any of a number of well-known methods.

Accordingly, in certain embodiments a sample comprises or consists of a purified or isolated polynucleotide, or it can comprise samples such as a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, sputum, ear flow, saliva or feces. In certain embodiments, the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, and the like, the “sample” expressly encompasses a processed fraction or portion derived from the biopsy, swab, smear, and the like.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent (e.g., HIV), and the like.

In one illustrative, but non-limiting embodiment, the sample is a maternal sample that is obtained from a pregnant female, for example a pregnant woman. In this instance, the sample can be analyzed using the methods described herein to provide a prenatal diagnosis of potential chromosomal abnormalities in the fetus. The maternal sample can be a tissue sample, a biological fluid sample, or a cell sample. A biological fluid includes, as non-limiting examples, blood, plasma, serum, sweat, tears, sputum, urine, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, and leukophoresis samples.

In another illustrative, but non-limiting embodiment, the maternal sample is a mixture of two or more biological samples, e.g., the biological sample can comprise two or more of a biological fluid sample, a tissue sample, and a cell culture sample. In some embodiments, the sample is a sample that is easily obtainable by non-invasive procedures, e.g., blood, plasma, serum, sweat, tears, sputum, urine, milk, sputum, ear flow, saliva and feces. In some embodiments, the biological sample is a peripheral blood sample, and/or the plasma and serum fractions thereof. In other embodiments, the biological sample is a swab or smear, a biopsy specimen, or a sample of a cell culture. As disclosed above, the terms “blood,” “plasma” and “serum” expressly encompass fractions or processed portions thereof. Similarly, where a sample is taken from a biopsy, swab, smear, and the like, the “sample” expressly encompasses a processed fraction or portion derived from the biopsy, swab, smear, and the like.

In certain embodiments, samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

Methods of isolating nucleic acids from biological sources are well known and will differ depending upon the nature of the source. One of skill in the art can readily isolate nucleic acid(s) from a source as needed for the method described herein. In some instances, it can be advantageous to fragment the nucleic acid molecules in the nucleic acid sample. Fragmentation can be random, or it can be specific, as achieved, for example, using restriction endonuclease digestion. Methods for random fragmentation are well known in the art, and include, for example, limited DNAse digestion, alkali treatment and physical shearing. In one embodiment, sample nucleic acids are obtained from as cfDNA, which is not subjected to fragmentation.

### Sequencing and Library Preparation

In some embodiments, nucleic acids (e.g., nucleic acid fragments, sample nucleic acid, test sample nucleic acid, cell-free nucleic acid, circulating cell-free nucleic acid) are sequenced. In some embodiments, a full or substantially full sequence is obtained and sometimes a partial sequence is obtained. In some embodiments, fragment length is determined using a sequencing method. In certain embodiments a non-targeted sequencing approach is used where most or all nucleic acids in a sample are sequenced, amplified and/or captured randomly. Certain aspects of sequencing and analysis processes are described hereafter.

In some embodiments, methods described herein utilize next generation sequencing technologies (NGS), that allow multiple samples to be sequenced individually as genomic molecules (i.e., singleplex sequencing) or as pooled samples comprising indexed genomic molecules (e.g., multiplex sequencing) on a single sequencing run. These methods can generate up to several hundred million reads of DNA sequences. In various embodiments, the sequences of genomic nucleic acids, and/or of indexed genomic nucleic acids can be determined using, for example, the Next Generation Sequencing Technologies (NGS) described herein. In various embodiments, analysis of the massive amount of sequence data obtained using NGS can be performed using one or more processors as described herein.

In various embodiments, the use of such sequencing technologies does not involve the preparation of sequencing libraries.

However, in certain embodiments, sequencing methods contemplated herein involve the preparation of sequencing libraries. A nucleic acid library (e.g., sequencing library) can be prepared by a suitable method as known in the art. A nucleic acid library can be prepared by a targeted or a non-targeted preparation process. In one illustrative approach, sequencing library preparation involves the production of a random collection of adapter-modified DNA fragments (e.g., polynucleotides) that are ready to be sequenced. Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originate in single-stranded form (e.g., ssDNA, RNA, and the like) and are converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), and the like), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

Preparation of sequencing libraries for some NGS sequencing platforms is facilitated by the use of polynucleotides comprising a specific range of fragment sizes. Preparation of such libraries typically involves the fragmentation of large polynucleotides (e.g., cellular genomic DNA) to obtain polynucleotides in the desired size range.

Fragmentation can be achieved by any of a number of methods known to those of skill in the art. For example, fragmentation can be achieved by mechanical means including, but not limited to nebulization, sonication and hydroshear. However mechanical fragmentation typically cleaves the DNA backbone at C—O, P—O and C—C bonds resulting in a heterogeneous mix of blunt and 3′- and 5′-overhanging ends with broken C—O, P—O and/C—C bonds which may need to be repaired as they may lack the requisite 5′-phosphate for the subsequent enzymatic reactions, e.g., ligation of sequencing adaptors, that are required for preparing DNA for sequencing.

In contrast, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3′-hydroxyl.

Standard protocols, e.g., protocols for sequencing using, for example, the Illumina platform as described elsewhere herein, instruct users to end-repair sample DNA, to purify the end-repaired products prior to dA-tailing, and to purify the dA-tailing products prior to the adaptor-ligating steps of the library preparation.

### Marker Nucleic Acids for Tracking and Verifying Sample Integrity

In various embodiments, verification of the integrity of the samples and sample tracking can be accomplished by sequencing mixtures of sample genomic nucleic acids, e.g., cfDNA, and accompanying marker nucleic acids that have been introduced into the samples, e.g., prior to processing.

Marker nucleic acids can be combined with the test sample (e.g., biological source sample) and subjected to processes that include, for example, one or more of the steps of fractionating the biological source sample, e.g., obtaining an essentially cell-free plasma fraction from a whole blood sample, purifying nucleic acids from a fractionated, e.g., plasma, or unfractionated biological source sample, e.g., a tissue sample, and sequencing. In some embodiments, sequencing comprises preparing a sequencing library. The sequence or combination of sequences of the marker molecules that are combined with a source sample is chosen to be unique to the source sample. In some embodiments, the unique marker molecules in a sample all have the same sequence. In other embodiments, the unique marker molecules in a sample are a plurality of sequences, e.g., a combination of two, three, four, five, six, seven, eight, nine, ten, fifteen, twenty, or more different sequences.

In one embodiment, the integrity of a sample can be verified using a plurality of marker nucleic acid molecules having identical sequences. Alternatively, the identity of a sample can be verified using a plurality of marker nucleic acid molecules that have at least two, at least three, at least four, at least five, at least six, at least seven, at least eight, at least nine, at least ten, at least 11, at least 12, at least 13, at least 14, at least 15, at least 16, at least 17 m, at least 18, at least 19, at least 20, at least 25, at least 30, at least 35, at least 40, at least 50, or more different sequences. Verification of the integrity of the plurality of biological samples, i.e., two or more biological samples, requires that each of the two or more samples be marked with marker nucleic acids that have sequences that are unique to each of the plurality of test sample that is being marked. For example, a first sample can be marked with a marker nucleic acid having sequence A, and a second sample can be marked with a marker nucleic acid having sequence B. Alternatively, a first sample can be marked with marker nucleic acid molecules all having sequence A, and a second sample can be marked with a mixture of sequences B and C, where sequences A, B and C are marker molecules having different sequences.

The marker nucleic acid(s) can be added to the sample at any stage of sample preparation that occurs prior to library preparation (if libraries are to be prepared) and sequencing. In one embodiment, marker molecules can be combined with an unprocessed source sample. For example, the marker nucleic acid can be provided in a collection tube that is used to collect a blood sample. Alternatively, the marker nucleic acids can be added to the blood sample following the blood draw. In one embodiment, the marker nucleic acid is added to the vessel that is used to collect a biological fluid sample, e.g., the marker nucleic acid(s) are added to a blood collection tube that is used to collect a blood sample. In another embodiment, the marker nucleic acid(s) are added to a fraction of the biological fluid sample. For example, the marker nucleic acid is added to the plasma and/or serum fraction of a blood sample, e.g., a maternal plasma sample. In yet another embodiment, the marker molecules are added to a purified sample, e.g., a sample of nucleic acids that have been purified from a biological sample. For example, the marker nucleic acid is added to a sample of purified maternal and fetal cfDNA. Similarly, the marker nucleic acids can be added to a biopsy specimen prior to processing the specimen. In some embodiments, the marker nucleic acids can be combined with a carrier that delivers the marker molecules into the cells of the biological sample. Cell-delivery carriers include pH-sensitive and cationic liposomes.

In various embodiments, the marker molecules have antigenomic sequences, that are sequences that are absent from the genome of the biological source sample. In an exemplary embodiment, the marker molecules that are used to verify the integrity of a human biological source sample have sequences that are absent from the human genome. In an alternative embodiment, the marker molecules have sequences that are absent from the source sample and from any one or more other known genomes. For example, the marker molecules that are used to verify the integrity of a human biological source sample have sequences that are absent from the human genome and from the mouse genome. The alternative allows for verifying the integrity of a test sample that comprises two or more genomes. For example, the integrity of a human cell-free DNA sample obtained from a subject affected by a pathogen, e.g., a bacterium, can be verified using marker molecules having sequences that are absent from both the human genome and the genome of the affecting bacterium. Sequences of genomes of numerous pathogens, e.g., bacteria, viruses, yeasts, fungi, protozoa and the like, are publicly available on the World Wide Web at ncbi.nlm.nih.gov/genomes. In another embodiment, marker molecules are nucleic acids that have sequences that are absent from any known genome. The sequences of marker molecules can be randomly generated algorithmically.

In various embodiments, the marker molecules can be naturally-occurring deoxyribonucleic acids (DNA), ribonucleic acids or artificial nucleic acid analogs (nucleic acid mimics) including peptide nucleic acids (PNA), morpholino nucleic acid, locked nucleic acids, glycol nucleic acids, and threose nucleic acids, which are distinguished from naturally-occurring DNA or RNA by changes to the backbone of the molecule or DNA mimics that do not have a phosphodiester backbone. The deoxyribonucleic acids can be from naturally-occurring genomes or can be generated in a laboratory through the use of enzymes or by solid phase chemical synthesis. Chemical methods can also be used to generate the DNA mimics that are not found in nature. Derivatives of DNA are that are available in which the phosphodiester linkage has been replaced but in which the deoxyribose is retained include but are not limited to DNA mimics having backbones formed by thioformacetal or a carboxamide linkage, which have been shown to be good structural DNA mimics. Other DNA mimics include morpholino derivatives and the peptide nucleic acids (PNA), which contain an N-(2-aminoethyl)glycine-based pseudopeptide backbone (Ann Rev Biophys Biomol Struct 24:167-183 [1995]). PNA is an extremely good structural mimic of DNA (or of ribonucleic acid [RNA]), and PNA oligomers are able to form very stable duplex structures with Watson-Crick complementary DNA and RNA (or PNA) oligomers, and they can also bind to targets in duplex DNA by helix invasion (Mol Biotechnol 26:233-248 [2004]. Another suitable structural mimic/analog of DNA analog that can be used as a marker molecule is phosphorothioate DNA in which one of the non-bridging oxygens is replaced by a sulfur. This modification reduces the action of endo- and exonucleases2 including 5′ to 3′ and 3′ to 5′ DNA POL 1 exonuclease, nucleases S1 and P1, RNases, serum nucleases and snake venom phosphodiesterase.

The length of the marker molecules can be distinct or indistinct from that of the sample nucleic acids, i.e., the length of the marker molecules can be similar to that of the sample genomic molecules, or it can be greater or smaller than that of the sample genomic molecules. The length of the marker molecules is measured by the number of nucleotide or nucleotide analog bases that constitute the marker molecule. Marker molecules having lengths that differ from those of the sample genomic molecules can be distinguished from source nucleic acids using separation methods known in the art. For example, differences in the length of the marker and sample nucleic acid molecules can be determined by electrophoretic separation, e.g., capillary electrophoresis. Size differentiation can be advantageous for quantifying and assessing the quality of the marker and sample nucleic acids. Preferably, the marker nucleic acids are shorter than the genomic nucleic acids, and of sufficient length to exclude them from being mapped to the genome of the sample. For example, as a 30-base human sequence is needed to uniquely map it to a human genome. Accordingly in certain embodiments, marker molecules used in sequencing bioassays of human samples should be at least 30 bp in length.

The choice of length of the marker molecule is determined primarily by the sequencing technology that is used to verify the integrity of a source sample. The length of the sample genomic nucleic acids being sequenced can also be considered. For example, some sequencing technologies employ clonal amplification of polynucleotides, which can require that the genomic polynucleotides that are to be clonally amplified be of a minimum length. For example, sequencing using the Illumina GAII sequence analyzer includes an in vitro clonal amplification by bridge PCR (also known as cluster amplification) of polynucleotides that have a minimum length of 110 bp, to which adaptors are ligated to provide a nucleic acid of at least 200 bp and less than 600 bp that can be clonally amplified and sequenced. In some embodiments, the length of the adaptor-ligated marker molecule is between about 200 bp and about 600 bp, between about 250 bp and 550 bp, between about 300 bp and 500 bp, or between about 350 and 450. In other embodiments, the length of the adaptor-ligated marker molecule is about 200 bp. For example, when sequencing fetal cfDNA that is present in a maternal sample, the length of the marker molecule can be chosen to be similar to that of fetal cfDNA molecules. Thus, in one embodiment, the length of the marker molecule used in an assay that comprises massively parallel sequencing of cfDNA in a maternal sample to determine the presence or absence of a fetal chromosomal aneuploidy, can be about 150 bp, about 160 bp, 170 bp, about 180 bp, about 190 bp or about 200 bp; preferably, the marker molecule is about 170 pp. Other sequencing approaches, e.g., SOLiD sequencing, Polony Sequencing and 454 sequencing use emulsion PCR to clonally amplify DNA molecules for sequencing, and each technology dictates the minimum and the maximum length of the molecules that are to be amplified. The length of marker molecules to be sequenced as clonally amplified nucleic acids can be up to about 600 bp. In some embodiments, the length of marker molecules to be sequenced can be greater than 600 bp.

Single molecule sequencing technologies, that do not employ clonal amplification of molecules, and are capable of sequencing nucleic acids over a very broad range of template lengths, in most situations do not require that the molecules to be sequenced be of any specific length. However, the yield of sequences per unit mass is dependent on the number of 3′ end hydroxyl groups, and thus having relatively short templates for sequencing is more efficient than having long templates. If starting with nucleic acids longer than 1000 nt, it is generally advisable to shear the nucleic acids to an average length of 100 to 200 nt so that more sequence information can be generated from the same mass of nucleic acids. Thus, the length of the marker molecule can range from tens of bases to thousands of bases. The length of marker molecules used for single molecule sequencing can be up to about 25 bp, up to about 50 bp, up to about 75 bp, up to about 100 bp, up to about 200 bp, up to about 300 bp, up to about 400 bp, up to about 500 bp, up to about 600 bp, up to about 700 bp, up to about 800 bp, up to about 900 bp, up to about 1000 bp, or more in length.

The length chosen for a marker molecule is also determined by the length of the genomic nucleic acid that is being sequenced. For example, cfDNA circulates in the human bloodstream as genomic fragments of cellular genomic DNA. Fetal cfDNA molecules found in the plasma of pregnant women are generally shorter than maternal cfDNA molecules (Chan et al., Clin Chem 50:8892 [2004]). Size fractionation of circulating fetal DNA has confirmed that the average length of circulating fetal DNA fragments is <300 bp, while maternal DNA has been estimated to be between about 0.5 and 1 Kb (Li et al., Clin Chem, 50: 1002-1011 [2004]). These findings are consistent with those of Fan et al., who determined using NGS that fetal cfDNA is rarely >340 bp (Fan et al., Clin Chem 56:1279-1286 [2010]). DNA isolated from urine with a standard silica-based method includes two fractions, high molecular weight DNA, which originates from shed cells, and low molecular weight (150-250 base pair) fraction of transrenal DNA (Tr-DNA) (Botezatu et al., Clin Chem. 46: 1078-1084, 2000; and Su et al., J Mol. Diagn. 6: 101-107, 2004). The application of newly developed technique for isolation of cell-free nucleic acids from body fluids to the isolation of transrenal nucleic acids has revealed the presence in urine of DNA and RNA fragments much shorter than 150 base pairs (U.S. Patent Application Publication No. 20080139801). In embodiments, where cfDNA is the genomic nucleic acid that is sequenced, marker molecules that are chosen can be up to about the length of the cfDNA. For example, the length of marker molecules used in maternal cfDNA samples to be sequenced as single nucleic acid molecules or as clonally amplified nucleic acids can be between about 100 bp and 600. In other embodiments, the sample genomic nucleic acids are fragments of larger molecules. For example, a sample genomic nucleic acid that is sequenced is fragmented cellular DNA. In embodiments, when fragmented cellular DNA is sequenced, the length of the marker molecules can be up to the length of the DNA fragments. In some embodiments, the length of the marker molecules is at least the minimum length required for mapping the sequence read uniquely to the appropriate reference genome. In other embodiments, the length of the marker molecule is the minimum length that is required to exclude the marker molecule from being mapped to the sample reference genome.

In addition, marker molecules can be used to verify samples that are not assayed by nucleic acid sequencing, and that can be verified by common bio-techniques other than sequencing, e.g., real-time PCR.

Sample Controls (e.g., in Process Positive Controls for Sequencing and/or Analysis)

In various embodiments, marker sequences introduced into the samples, e.g., as described above, can function as positive controls to verify the accuracy and efficacy of sequencing and subsequent processing and analysis.

Accordingly, compositions and method for providing an in-process positive control (IPC) for sequencing DNA in a sample are provided. In certain embodiments, positive controls are provided for sequencing cfDNA in a sample comprising a mixture of genomes are provided. An IPC can be used to relate baseline shifts in sequence information obtained from different sets of samples, e.g., samples that are sequenced at different times on different sequencing runs. Thus, for example, an IPC can relate the sequence information obtained for a maternal test sample to the sequence information obtained from a set of qualified samples that were sequenced at a different time.

Similarly, in the case of segment analysis, an IPC can relate the sequence information obtained from a subject for particular segment(s) to the sequence obtained from a set of qualified samples (of similar sequences) that were sequenced at a different time. In certain embodiments, an IPC can relate the sequence information obtained from a subject for particular cancer-related loci to the sequence information obtained from a set of qualified samples (e.g., from a known amplification/deletion, and the like).

In addition, IPCs can be used as markers to track sample(s) through the sequencing process. IPCs can also provide a qualitative positive sequence dose value, e.g., NCV, for one or more aneuploidies of chromosomes of interest, e.g., trisomy 21, trisomy 13, trisomy 18 to provide proper interpretation, and to ensure the dependability and accuracy of the data. In certain embodiments, IPCs can be created to comprise nucleic acids from male and female genomes to provide doses for chromosomes X and Y in a maternal sample to determine whether the fetus is male.

The type and the number of in-process controls depends on the type or nature of the test needed. For example, for a test requiring the sequencing of DNA from a sample comprising a mixture of genomes to determine whether a chromosomal aneuploidy exists, the in-process control can comprise DNA obtained from a sample known comprising the same chromosomal aneuploidy that is being tested. In some embodiments, the IPC includes DNA from a sample known to comprise an aneuploidy of a chromosome of interest. For example, the IPC for a test to determine the presence or absence of a fetal trisomy, e.g., trisomy 21, in a maternal sample comprises DNA obtained from an individual with trisomy 21. In some embodiments, the IPC comprises a mixture of DNA obtained from two or more individuals with different aneuploidies. For example, for a test to determine the presence or absence of trisomy 13, trisomy 18, trisomy 21, and monosomy X, the IPC comprises a combination of DNA samples obtained from pregnant women each carrying a fetus with one of the trisomies being tested. In addition to complete chromosomal aneuploidies, IPCs can be created to provide positive controls for tests to determine the presence or absence of partial aneuploidies.

An IPC that serves as the control for detecting a single aneuploidy can be created using a mixture of cellular genomic DNA obtained from two subjects, one being the contributor of the aneuploid genome. For example, an IPC that is created as a control for a test to determine a fetal trisomy, e.g., trisomy 21, can be created by combining genomic DNA from a male or female subject carrying the trisomic chromosome with genomic DNA with a female subject known not to carry the trisomic chromosome. Genomic DNA can be extracted from cells of both subjects, and sheared to provide fragments of between about 100-400 bp, between about 150-350 bp, or between about 200-300 bp to simulate the circulating cfDNA fragments in maternal samples. The proportion of fragmented DNA from the subject carrying the aneuploidy, e.g., trisomy 21, is chosen to simulate the proportion of circulating fetal cfDNA found in maternal samples to provide an IPC comprising a mixture of fragmented DNA comprising about 5%, about 10%, about 15%, about 20%, about 25%, about 30%, of DNA from the subject carrying the aneuploidy. The IPC can comprise DNA from different subjects each carrying a different aneuploidy. For example, the IPC can comprise about 80% of the unaffected female DNA, and the remaining 20% can be DNA from three different subjects each carrying a trisomic chromosome 21, a trisomic chromosome 13, and a trisomic chromosome 18. The mixture of fragmented DNA is prepared for sequencing. Processing of the mixture of fragmented DNA can comprise preparing a sequencing library, which can be sequenced using any massively parallel methods in singleplex or multiplex fashion. Stock solutions of the genomic IPC can be stored and used in multiple diagnostic tests.

Alternatively, the IPC can be created using cfDNA obtained from a mother known to carry a fetus with a known chromosomal aneuploidy. For example, cfDNA can be obtained from a pregnant woman carrying a fetus with trisomy 21. The cfDNA is extracted from the maternal sample, and cloned into a bacterial vector and grown in bacteria to provide an ongoing source of the IPC. The DNA can be extracted from the bacterial vector using restriction enzymes. Alternatively, the cloned cfDNA can be amplified by, e.g., PCR. The IPC DNA can be processed for sequencing in the same runs as the cfDNA from the test samples that are to be analyzed for the presence or absence of chromosomal aneuploidies.

While the creation of IPCs is described above with respect to trisomies, it will be appreciated that IPCs can be created to reflect other partial aneuploidies including for example, various segment amplification and/or deletions. Thus, for example, where various cancers are known to be associated with particular amplifications (e.g., breast cancer associated with 20Q13) IPCs can be created that incorporate those known amplifications.

### Sequencing Methods

As indicated above, the prepared samples (e.g., sequencing libraries) are sequenced as part of the procedure for identifying copy number variation(s). Any of a number of sequencing technologies can be utilized, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequences. In certain embodiments a non-targeted MPS approach is used where most or all nucleic acids in a sample are sequenced, amplified and/or captured randomly. With certain sequencing technologies (e.g., MPS sequencing methods), thousands to millions of nucleic acid (e.g. DNA) fragments can be sequenced in parallel.

Some sequencing technologies are available commercially, such as the sequencing-by-hybridization platform from Affymetrix Inc. (Sunnyvale, CA) and the sequencing-by-synthesis platforms from 454 Life Sciences (Bradford, CT), Illumina/Solexa (Hayward, CA) and Helicos Biosciences (Cambridge, MA), and the sequencing-by-ligation platform from Applied Biosystems (Foster City, CA), as described below. In addition to the single molecule sequencing performed using sequencing-by-synthesis of Helicos Biosciences, other single molecule sequencing technologies include, but are not limited to, the SMRT™ technology of Pacific Biosciences, the ION TORRENT™ technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM). Illustrative sequencing technologies are described in greater detail below.

In one illustrative, but non-limiting, embodiment, the methods described herein comprise obtaining sequence information for the nucleic acids in a test sample, e.g., cfDNA in a maternal sample, cfDNA or cellular DNA in a subject being screened for a cancer, and the like, using Illumina's sequencing-by-synthesis and reversible terminator-based sequencing chemistry (e.g., as described in Bentley et al., Nature 6:53-59 [2009]). Template DNA can be genomic DNA, e.g., cellular DNA or cfDNA. In some embodiments, genomic DNA from isolated cells is used as the template, and it is fragmented into lengths of several hundred base pairs. In other embodiments, cfDNA is used as the template, and fragmentation is not required as cfDNA exists as short fragments. For example, fetal cfDNA circulates in the bloodstream as fragments approximately 170 base pairs (bp) in length (Fan et al., Clin Chem 56:1279-1286 [2010]), and no fragmentation of the DNA is required prior to sequencing. Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchor oligos (not to be confused with the anchor/anchored reads in the analysis of repeat expansion). Under limiting-dilution conditions, adapter-modified, single-stranded template DNA is added to the flow cell and immobilized by hybridization to the anchor oligos. Attached DNA fragments are extended and bridge amplified to create an ultra-high density sequencing flow cell with hundreds of millions of clusters, each containing about 1,000 copies of the same template. In one embodiment, the randomly fragmented genomic DNA is amplified using PCR before it is subjected to cluster amplification. Alternatively, an amplification-free (e.g., PCR free) genomic library preparation is used, and the randomly fragmented genomic DNA is enriched using the cluster amplification alone (Kozarewa et al., Nature Methods 6:291-295 [2009]). The templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about tens to a few hundred base pairs are aligned against a reference genome and unique mapping of the short sequence reads to the reference genome are identified using specially developed data analysis pipeline software. After completion of the first read, the templates can be regenerated in situ to enable a second read from the opposite end of the fragments. Thus, either single-end or paired end sequencing of the DNA fragments can be used.

Various embodiments of the disclosure may use sequencing by synthesis that allows paired end sequencing. In some embodiments, the sequencing by synthesis platform by Illumina involves clustering fragments. Clustering is a process in which each fragment molecule is isothermally amplified. In some embodiments, as the example described here, the fragment has two different adaptors attached to the two ends of the fragment, the adaptors allowing the fragment to hybridize with the two different oligos on the surface of a flow cell lane. The fragment further includes or is connected to two index sequences at two ends of the fragment, which index sequences provide labels to identify different samples in multiplex sequencing. In some sequencing platforms, a fragment to be sequenced is also referred to as an insert.

In some implementations, a flow cell for clustering in the Illumina platform is a glass slide with lanes. Each lane is a glass channel coated with a lawn of two types of oligos. Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification, a strand folds over, and a second adapter region on a second end of the strand hybridizes with the second type of oligos on the flow cell surface. A polymerase generates a complimentary strand, forming a double-stranded bridge molecule. This double-stranded molecule is denatured resulting in two single-stranded molecules tethered to the flow cell through two different oligos. The process is then repeated over and over, and occurs simultaneously for millions of clusters resulting in clonal amplification of all the fragments. After bridge amplification, the reverse strands are cleaved and washed off, leaving only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters are sequenced in a massively parallel manner. At the completion of the first read, the read product is washed away.

In the next step of protocols involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flow cell. An index 2 sequence is read in the same manner as index 1. Then an index 2 read product is washed off at the completion of the step.

After reading two indices, read 2 initiates by using polymerases to extend the second flow cell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences. These contiguous sequences are aligned to the reference genome for variant identification.

The sequencing by synthesis example described above involves paired end reads, which is used in many of the embodiments of the disclosed methods. Paired end sequencing involves 2 reads from the two ends of a fragment. When a pair of reads are mapped to a reference sequence, the base-pair distance between the two reads can be determined, which distance can then be used to determine the length of the fragments from which the reads were obtained. In some instances, a fragment straddling two bins would have one of its pair-end read aligned to one bin, and another to an adjacent bin. This gets rarer as the bins get longer or the reads get shorter. Various methods may be used to account for the bin-membership of these fragments. For instance, they can be omitted in determining fragment size frequency of a bin; they can be counted for both of the adjacent bins; they can be assigned to the bin that encompasses the larger number of base pairs of the two bins; or they can be assigned to both bins with a weight related to portion of base pairs in each bin.

Paired end reads may use inserts of different lengths (i.e., different fragment sizes to be sequenced). As the default meaning in this disclosure, paired end reads are used to refer to reads obtained from various insert lengths. In some instances, to distinguish short-insert paired end reads from long-inserts paired end reads, the latter is also referred to as mate pair reads. In some embodiments involving mate pair reads, two biotin junction adaptors first are attached to two ends of a relatively long insert (e.g., several kb). The biotin junction adaptors then link the two ends of the insert to form a circularized molecule. A sub-fragment encompassing the biotin junction adaptors can then be obtained by further fragmenting the circularized molecule. The sub-fragment including the two ends of the original fragment in opposite sequence order can then be sequenced by the same procedure as for short-insert paired end sequencing described above. Further details of mate pair sequencing using an Illumina platform is shown in an online publication at the following URL, which is incorporated by reference by its entirety: res|.|illumina|.com/documents/products/technotes/technote_nextera_matepair_data_processing.

Additional information about paired end sequencing can be found in U.S. Pat. No. 7,601,499 and US Patent Publication No. 2012/0,053,063, which are incorporated by reference with regard to materials on paired end sequencing methods and apparatuses.

After sequencing of DNA fragments, sequence reads of predetermined length, e.g., 100 bp, are mapped or aligned to a known reference genome. The mapped or aligned reads and their corresponding locations on the reference sequence are also referred to as tags. In one embodiment, the reference genome sequence is the NCBI36/hg18 sequence, which is available on the world wide web at genome dot ucsc dot edu/cgi-bin/hgGateway?org=Human&db=hg18&hgsid=166260105). Alternatively, the reference genome sequence is the GRCh37/hg19, which is available on the world wide web at genome dot ucsc dot edu/cgi-bin/hgGateway. Other sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al., Genome Biology 10:R25.1-R25.10 [2009]), or ELAND (Illumina, Inc., San Diego, CA, USA). In one embodiment, one end of the clonally expanded copies of the plasma cfDNA molecules is sequenced and processed by bioinformatics alignment analysis for the Illumina Genome Analyzer, which uses the Efficient Large-Scale Alignment of Nucleotide Databases (ELAND) software.

In one illustrative, but non-limiting, embodiment, the methods described herein comprise obtaining sequence information for the nucleic acids in a test sample, e.g., cfDNA in a maternal sample, cfDNA or cellular DNA in a subject being screened for a cancer, and the like, using single molecule sequencing technology of the Helicos True Single Molecule Sequencing (tSMS) technology (e.g., as described in Harris T. D. et al., Science 320:106-109 [2008]). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In certain embodiments, the templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

In another illustrative, but non-limiting embodiment, the methods described herein comprise obtaining sequence information for the nucleic acids in the test sample, e.g., cfDNA in a maternal test sample, cfDNA or cellular DNA in a subject being screened for a cancer, and the like, using the 454 sequencing (Roche) (e.g., as described in Margulies, M. et al. Nature 437:376-380 [2005]). 454 sequencing typically involves two steps. In the first step, DNA is sheared into fragments of approximately 300-800 base pairs, and the fragments are blunt-ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (e.g., picoliter-sized wells). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is measured and analyzed.

In another illustrative, but non-limiting, embodiment, the methods described herein comprises obtaining sequence information for the nucleic acids in the test sample, e.g., cfDNA in a maternal test sample, cfDNA or cellular DNA in a subject being screened for a cancer, and the like, using the SOLiD™ technology (Applied Biosystems). In SOLiD™ sequencing-by-ligation, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In another illustrative, but non-limiting, embodiment, the methods described herein comprise obtaining sequence information for the nucleic acids in the test sample, e.g., cfDNA in a maternal test sample, cfDNA or cellular DNA in a subject being screened for a cancer, and the like, using the single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector comprises a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In another illustrative, but non-limiting embodiment, the methods described herein comprise obtaining sequence information for the nucleic acids in the test sample, e.g., cfDNA in a maternal test sample, cfDNA or cellular DNA in a subject being screened for a cancer, and the like, using nanopore sequencing (e.g., as described in Soni G V and Meller A. Clin Chem 53: 1996-2001 [2007]). Nanopore sequencing DNA analysis techniques are developed by a number of companies, including, for example, Oxford Nanopore Technologies (Oxford, United Kingdom), Sequenom, NABsys, and the like. Nanopore sequencing is a single-molecule sequencing technology whereby a single molecule of DNA is sequenced directly as it passes through a nanopore. A nanopore is a small hole, typically of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore provides a read of the DNA sequence.

In another illustrative, but non-limiting, embodiment, the methods described herein comprises obtaining sequence information for the nucleic acids in the test sample, e.g., cfDNA in a maternal test sample, cfDNA or cellular DNA in a subject being screened for a cancer, and the like, using the chemical-sensitive field effect transistor (chemFET) array (e.g., as described in U.S. Patent Application Publication No. 2009/0026082). In one example of this technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned as a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In another embodiment, the present method comprises obtaining sequence information for the nucleic acids in the test sample, e.g., cfDNA in a maternal test sample, using transmission electron microscopy (TEM). The method, termed Individual Molecule Placement Rapid Nano Transfer (IMPRNT), comprises utilizing single atom resolution transmission electron microscope imaging of high-molecular weight (150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA. The method is further described in PCT patent publication WO 2009/046445. The method allows for sequencing complete human genomes in less than ten minutes.

In another embodiment, the DNA sequencing technology is the Ion Torrent single molecule sequencing, which pairs semiconductor technology with a simple sequencing chemistry to directly translate chemically encoded information (A, C, G, T) into digital information (0, 1) on a semiconductor chip. In nature, when a nucleotide is incorporated into a strand of DNA by a polymerase, a hydrogen ion is released as a byproduct. Ion Torrent uses a high-density array of micro-machined wells to perform this biochemical process in a massively parallel way. Each well holds a different DNA molecule. Beneath the wells is an ion-sensitive layer and beneath that an ion sensor. When a nucleotide, for example a C, is added to a DNA template and is then incorporated into a strand of DNA, a hydrogen ion will be released. The charge from that ion will change the pH of the solution, which can be detected by Ion Torrent's ion sensor. The sequencer—essentially the world's smallest solid-state pH meter—calls the base, going directly from chemical information to digital information. The Ion personal Genome Machine (PGM™) sequencer then sequentially floods the chip with one nucleotide after another. If the next nucleotide that floods the chip is not a match. No voltage change will be recorded and no base will be called. If there are two identical bases on the DNA strand, the voltage will be double, and the chip will record two identical bases called. Direct detection allows recordation of nucleotide incorporation in seconds.

In another embodiment, the present method comprises obtaining sequence information for the nucleic acids in the test sample, e.g., cfDNA in a maternal test sample, using sequencing by hybridization. Sequencing-by-hybridization comprises contacting the plurality of polynucleotide sequences with a plurality of polynucleotide probes, where each of the plurality of polynucleotide probes can be optionally tethered to a substrate. The substrate might be flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be determined and used to identify the plurality of polynucleotide sequences within the sample.

In some embodiments of the methods described herein, the mapped sequence tags comprise sequence reads of about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. It is expected that technological advances will enable single-end reads of greater than 500 bp enabling for reads of greater than about 1000 bp when paired end reads are generated. In one embodiment, the mapped sequence tags comprise sequence reads that are 36 bp. Mapping of the sequence tags is achieved by comparing the sequence of the tag with the sequence of the reference to determine the chromosomal origin of the sequenced nucleic acid (e.g., cfDNA) molecule, and specific genetic sequence information is not needed. A small degree of mismatch (0-2 mismatches per sequence tag) may be allowed to account for minor polymorphisms that may exist between the reference genome and the genomes in the mixed sample.

A plurality of sequence tags is typically obtained per sample. In some embodiments, at least about 3×10⁶ sequence tags, at least about 5×10⁶ sequence tags, at least about 8×10⁶ sequence tags, at least about 10×10⁶ sequence tags, at least about 15×10⁶ sequence tags, at least about 20×10⁶ sequence tags, at least about 30×10⁶ sequence tags, at least about 40×10⁶ sequence tags, or at least about 50×10⁶ sequence tags comprising between 20 and 40 bp reads, e.g., 36 bp, are obtained from mapping the reads to the reference genome per sample. In one embodiment, all the sequence reads are mapped to all regions of the reference genome. In one embodiment, the tags that have been mapped to all regions, e.g., all chromosomes, of the reference genome are counted, and the CNV, i.e., the over- or under-representation of a sequence of interest, e.g., a chromosome or portion thereof, in the mixed DNA sample is determined. The method does not require differentiation between the two genomes.

The accuracy required for correctly determining whether a CNV, e.g., aneuploidy, is present or absent in a sample, is predicated on the variation of the number of sequence tags that map to the reference genome among samples within a sequencing run (inter-chromosomal variability), and the variation of the number of sequence tags that map to the reference genome in different sequencing runs (inter-sequencing variability). For example, the variations can be particularly pronounced for tags that map to GC-rich or GC-poor reference sequences. Other variations can result from using different protocols for the extraction and purification of the nucleic acids, the preparation of the sequencing libraries, and the use of different sequencing platforms. The present method uses sequence doses (chromosome doses, or segment doses) based on the knowledge of normalizing sequences (normalizing chromosome sequences or normalizing segment sequences), to intrinsically account for the accrued variability stemming from interchromosomal (intra-run), and inter-sequencing (inter-run) and platform-dependent variability. Chromosome doses are based on the knowledge of a normalizing chromosome sequence, which can be composed of a single chromosome, or of two or more chromosomes selected from chromosomes 1-22, X, and Y. Alternatively, normalizing chromosome sequences can be composed of a single chromosome segment, or of two or more segments of one chromosome or of two or more chromosomes. Segment doses are based on the knowledge of a normalizing segment sequence, which can be composed of a single segment of any one chromosome, or of two or more segments of any two or more of chromosomes 1-22, X, and Y.

### Mapping Reads

In some embodiments, sequence reads are mapped to a genome (e.g., a reference genome) or part thereof. Any suitable mapping method (e.g., process, algorithm, program, software, module, the like or combination thereof) can be used. Certain aspects of mapping processes are described hereafter.

Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped”, “a mapped sequence read”, “a mapped read”, or a “sequence tag”. In certain embodiments, a mapped sequence read is referred to as a “hit” or “count”. In some embodiments, mapped sequence reads are grouped together according to various parameters, as described herein. In some embodiments, mapped sequence reads are assigned to particular chromosomes or portions thereof.

As used herein, the terms “aligned”, “alignment”, or “aligning” refer to two or more nucleic acid sequences that can be identified as a match (e.g., 100% identity) or partial match. Alignments can be done manually or by a computer (e.g., a software, program, module, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (i.e., non-perfect match, partial match, partial alignment). In some embodiments, an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand. In certain embodiments a nucleic acid sequence is aligned with the reverse complement of another nucleic acid sequence.

Various computational methods can be used to map each sequence read to a reference genome, bin, or portion thereof. Non-limiting examples of computer algorithms that can be used to align sequences include, without limitation, BLAST, BLITZ, FASTA, BOWTIE 1, BOWTIE 2, ELAND, MAQ, PROBEMATCH, SOAP or SEQMAP, or variations thereof or combinations thereof. In some embodiments, sequence reads can be aligned with sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database. Search hits can then be used to sort the identified sequences into appropriate chromosomes, genomic portions or bins, for example.

In some embodiments, a read may uniquely or non-uniquely map to a reference genome, bin, or portion thereof. A read is considered as “uniquely mapped” if it aligns with a single sequence in the reference genome. A read is considered as “non-uniquely mapped” if it aligns with two or more sequences in the reference genome. In some embodiments, non-uniquely mapped reads are eliminated from further analysis (e.g., fragment length measurements, sequence motif quantifications). A certain, small degree of mismatch (0-1) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. For example, a reference genome used for human subjects as well as many other organisms can be found at the National Center for Biotechnology Information at www.ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, where a sample nucleic acid is from a pregnant subject, a reference sequence sometimes is not from the fetus, the mother of the fetus or the father of the fetus, and is referred to herein as an “external reference.” A maternal reference may be prepared and used in some embodiments. When a reference from the pregnant female is prepared (“maternal reference sequence”) based on an external reference, reads from DNA of the pregnant female that contains substantially no fetal DNA often are mapped to the external reference sequence and assembled. In certain embodiments the external reference is from DNA of an individual having substantially the same ethnicity as the pregnant female. A maternal reference sequence may not completely cover the maternal genomic DNA (e.g., it may cover about 50%, 60%, 70%, 80%, 90% or more of the maternal genomic DNA), and the maternal reference may not perfectly match the maternal genomic DNA sequence (e.g., the maternal reference sequence may include multiple mismatches).

In certain embodiments, mappability is assessed for a reference genome or genomic region (e.g., portion, genomic portion, bin). Mappability is the ability to unambiguously align a nucleotide sequence read to a reference genome or a portion of a reference genome or a bin, typically up to a specified number of mismatches, including, for example, 0, 1, 2 or more mismatches. For a given genomic region, the expected mappability can be estimated using a sliding-window approach of a preset read length and averaging the resulting read-level mappability values. Genomic regions comprising stretches of unique nucleotide sequence sometimes have a high mappability value.

### Sequence Read Quantifications

Sequence reads that are mapped or partitioned based on a selected feature or variable can be quantified to determine the number of reads that are mapped to one or more bins, portions, sections, partitions, loci (e.g., bins, portions, sections, partitions, loci of a reference genome), in some embodiments. In some embodiments, a number of sequence reads or sequence tags aligning to a bin in a sequence of interest is determined. In some embodiments, a number of sequence reads or sequence tags aligning to each bin in a plurality of bins in a sequence of interest is determined. In certain embodiments, the quantity of sequence reads that are mapped to a bin are termed counts (e.g., a count). Often a count is associated with a bin. In certain embodiments counts for two or more bins (e.g., a set of bins) are mathematically manipulated (e.g., averaged, added, normalized, the like or a combination thereof). In some embodiments a count is determined from some or all of the sequence reads mapped to (i.e., associated with) a bin. In certain embodiments, a count is determined from a pre-defined subset of mapped sequence reads. Pre-defined subsets of mapped sequence reads can be defined or selected utilizing any suitable feature or variable. In some embodiments, pre-defined subsets of mapped sequence reads can include from 1 to n sequence reads, where n represents a number equal to the sum of all sequence reads generated from a test subject or reference subject sample.

In certain embodiments, a sequence read quantification or count is derived from sequence reads that are processed or manipulated by a suitable method, operation or mathematical process known in the art. A quantification or count can be determined by a suitable method, operation or mathematical process. In certain embodiments a quantification or count is derived from sequence reads associated with a bin where some or all of the sequence reads are weighted, removed, filtered, normalized, adjusted, averaged, derived as a mean, added, or subtracted or processed by a combination thereof. In some embodiments, a quantification or count is derived from raw sequence reads and or filtered sequence reads. In certain embodiments, a quantification or count value is determined by a mathematical process. In certain embodiments, a quantification or count value is an average, mean or sum of sequence reads mapped to a bin. In some embodiments, a quantification or count is a mean number of counts. In some embodiments, a quantification or count is associated with an uncertainty value.

In some embodiments, sequence read quantifications or counts can be manipulated or transformed (e.g., normalized, combined, added, filtered, selected, averaged, derived as a mean, the like, or a combination thereof). In some embodiments, quantifications or counts can be transformed to produce normalized counts. Quantifications or counts can be processed (e.g., normalized) by a method known in the art and/or as described herein (e.g., bin-wise normalization, normalization by GC content, linear and nonlinear least squares regression, GC LOESS, LOWESS, RM, GCRM, cQn and/or combinations thereof).

Sequence read quantifications or counts may be obtained from a nucleic acid sample from a pregnant subject bearing a fetus. Quantifications or counts of nucleic acid sequence reads mapped to one or more bins often are quantifications or counts representative of both the fetus and the mother of the fetus (e.g., a pregnant subject). In certain embodiments, some of the quantifications or counts mapped to a bin are from a fetal genome and some of the counts mapped to the same bin are from a maternal genome.

### Data Processing

Measured features for a test sample may include fragment lengths, fragment length ratios, sequence read counts, sequence tag counts, and the like and may be referred to herein as raw data, since the data represents unmanipulated quantifications of such features. In some embodiments, measured feature data in a data set can be processed further (e.g., mathematically and/or statistically manipulated) and/or displayed to facilitate providing an outcome. In certain embodiments, data sets, including larger data sets, may benefit from pre-processing to facilitate further analysis. Pre-processing of data sets sometimes involves removal of redundant and/or uninformative data. Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. The terms “pre-processing” and “processing” when utilized with respect to data or data sets are collectively referred to herein as “processing”. Processing can render data more amenable to further analysis, and can generate an outcome in some embodiments. In some embodiments, one or more or all processing methods are performed by a processor, a micro-processor, a computer, in conjunction with memory and/or by a microprocessor-controlled machine.

The term “noisy data” as used herein refers to (a) data that has a significant variance between data points when analyzed or plotted, (b) data that has a significant standard deviation (e.g., greater than 3 standard deviations), (c) data that has a significant standard error of the mean, the like, and combinations of the foregoing. Noisy data sometimes occurs due to the quantity and/or quality of starting material (e.g., nucleic acid sample), and sometimes occurs as part of processes for preparing or replicating DNA used to generate sequence reads. In certain embodiments, noise results from certain sequences being over represented when prepared using PCR-based methods.

The term “uninformative data” as used herein refer to data having a numerical value that is significantly different from a predetermined threshold value or falls outside a predetermined cutoff range of values. A threshold value or range of values often is calculated by mathematically and/or statistically manipulating data (e.g., from a reference and/or subject). In some embodiments, an uncertainty value is determined. An uncertainty value generally is a measure of variance or error and can be any suitable measure of variance or error. In some embodiments, an uncertainty value is a standard deviation, standard error, calculated variance, p-value, or mean absolute deviation (MAD).

Any suitable procedure can be utilized for processing data sets described herein. Non-limiting examples of procedures suitable for use for processing data sets include filtering, normalizing, weighting, monitoring peak heights, monitoring peak areas, monitoring peak edges, determining area ratios, mathematical processing of data, statistical processing of data, application of statistical algorithms, analysis with fixed variables, analysis with optimized variables, plotting data to identify patterns or trends for additional processing, the like, and combinations of the foregoing. In some embodiments, data sets are processed based on various features (e.g., GC content, redundant mapped reads, centromere regions, telomere regions, the like, and combinations thereof) and/or variables (e.g., fetal gender, maternal age, maternal ploidy, percent contribution of fetal nucleic acid, the like, or combinations thereof). In certain embodiments, processing data sets as described herein can reduce the complexity and/or dimensionality of large and/or complex data sets. In some embodiments, data sets can include from thousands to millions of sequence reads, fragment lengths, fragment length ratios, and the like for each test sample.

Data processing can be performed in any number of steps, in certain embodiments. For example, data may be processed using only a single processing procedure, in some embodiments, and in certain embodiments, data may be processed using 1 or more, 5 or more, 10 or more or 20 or more processing steps (e.g., 1 or more processing steps, 2 or more processing steps, 3 or more processing steps, 4 or more processing steps, 5 or more processing steps, 6 or more processing steps, 7 or more processing steps, 8 or more processing steps, 9 or more processing steps, 10 or more processing steps, 11 or more processing steps, 12 or more processing steps, 13 or more processing steps, 14 or more processing steps, 15 or more processing steps, 16 or more processing steps, 17 or more processing steps, 18 or more processing steps, 19 or more processing steps, or 20 or more processing steps). In some embodiments, processing steps may be the same step repeated two or more times, and in certain embodiments, processing steps may be two or more different processing steps, carried out simultaneously or sequentially. In some embodiments, any suitable number and/or combination of the same or different processing steps can be utilized to process sequence read data to facilitate providing an outcome.

In certain embodiments, processing data sets by the criteria described herein may reduce the complexity and/or dimensionality of a data set. For example, a principal component analysis (PCA) can reduce the complexity and/or dimensionality of a data set. A PCA can be performed by a suitable PCA method, or a variation thereof. Non-limiting examples of a PCA method include a canonical correlation analysis (CCA), a Karhunen-Loeve transform (KLT), a Hotelling transform, a proper orthogonal decomposition (POD), a singular value decomposition (SVD) of X, an eigenvalue decomposition (EVD) of XTX, a factor analysis, an Eckart-Young theorem, a Schmidt-Mirsky theorem, empirical orthogonal functions (EOF), an empirical eigenfunction decomposition, an empirical component analysis, quasiharmonic modes, a spectral decomposition, an empirical modal analysis, the like, variations or combinations thereof. A PCA often identifies one or more principal components. In some embodiments, a PCA identifies a 1^{st}, 2^{nd}, 3^{rd}, 4^{th}, 5^{th}, 6^{th}, 7^{th}, 8^{th}, 9^{th}, and a 10^{th} or more principal components.

In some embodiments, one or more processing steps can comprise one or more normalization steps. Normalization can be performed by a suitable method described herein or known in the art. In certain embodiments, normalization comprises adjusting values measured on different scales to a notionally common scale. In certain embodiments, normalization comprises a sophisticated mathematical adjustment to bring probability distributions of adjusted values into alignment. In some embodiments, normalization comprises aligning distributions to a normal distribution. In certain embodiments, normalization comprises mathematical adjustments that allow comparison of corresponding normalized values for different datasets in a way that eliminates the effects of certain gross influences (e.g., error and anomalies). In certain embodiments, normalization comprises scaling. Normalization sometimes comprises division of one or more data sets by a predetermined variable or formula.

Any suitable number of normalizations can be used. In some embodiments, data sets can be normalized 1 or more, 5 or more, 10 or more or even 20 or more times. Data sets can be normalized to values (e.g., normalizing value) representative of any suitable feature or variable (e.g., sample data, reference data, or both). Normalizing a data set sometimes has the effect of isolating statistical error, depending on the feature or property selected as the predetermined normalization variable. Normalizing a data set sometimes also allows comparison of data characteristics of data having different scales, by bringing the data to a common scale (e.g., predetermined normalization variable). In some embodiments, one or more normalizations to a statistically derived value can be utilized to minimize data differences and diminish the importance of outlying data.

In some embodiments, a processing step comprises a weighting. The terms “weighted”, “weighting” or “weight function” or grammatical derivatives or equivalents thereof, as used herein, refer to a mathematical manipulation of a portion or all of a data set sometimes utilized to alter the influence of certain data set features or variables with respect to other data set features or variables. A weighting function can be used to increase the influence of data with a relatively small measurement variance, and/or to decrease the influence of data with a relatively large measurement variance, in some embodiments. A non-limiting example of a weighting function is [1/(standard deviation)²]. A weighting step sometimes is performed in a manner substantially similar to a normalizing step. In some embodiments, a data set is divided by a predetermined variable (e.g., weighting variable). A predetermined variable (e.g., minimized target function, Phi) often is selected to weigh different parts of a data set differently (e.g., increase the influence of certain data types while decreasing the influence of other data types).

In certain embodiments, a processing step can comprise one or more mathematical and/or statistical manipulations. Any suitable mathematical and/or statistical manipulation, alone or in combination, may be used to analyze and/or manipulate a data set described herein. Any suitable number of mathematical and/or statistical manipulations can be used. In some embodiments, a data set can be mathematically and/or statistically manipulated 1 or more, 5 or more, 10 or more or 20 or more times. Non-limiting examples of mathematical and statistical manipulations that can be used include addition, subtraction, multiplication, division, algebraic functions, least squares estimators, curve fitting, differential equations, rational polynomials, double polynomials, orthogonal polynomials, z-scores, p-values, chi values, phi values, analysis of peak levels, determination of peak edge locations, calculation of peak area ratios, analysis of median chromosomal level, calculation of mean absolute deviation, sum of squared residuals, mean, standard deviation, standard error, the like or combinations thereof. Non-limiting examples of data set variables or features that can be statistically manipulated include fragment lengths, fragment length ratios, sequence read quantifications, fetal fraction, the like or combinations thereof.

In some embodiments, a processing step can comprise the use of one or more statistical algorithms. Any suitable statistical algorithm, alone or in combination, may be used to analyze and/or manipulate a data set described herein. Any suitable number of statistical algorithms can be used. In some embodiments, a data set can be analyzed using 1 or more, 5 or more, 10 or more or 20 or more statistical algorithms. Non-limiting examples of statistical algorithms suitable for use with methods described herein include decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. Non-limiting examples of data set variables or features that can be analyzed using statistical algorithms include fragment lengths, fragment length ratios, sequence read quantifications, fetal fraction, the like or combinations thereof.

In certain embodiments, a data set can be analyzed by utilizing multiple (e.g., 2 or more) statistical algorithms (e.g., least squares regression, principle component analysis, linear discriminant analysis, quadratic discriminant analysis, bagging, neural networks, support vector machine models, random forests, classification tree models, K-nearest neighbors, logistic regression and/or loss smoothing) and/or mathematical and/or statistical manipulations (e.g., referred to herein as manipulations). The use of multiple manipulations can generate an N-dimensional space that can be used to provide an outcome, in some embodiments. In certain embodiments, analysis of a data set by utilizing multiple manipulations can reduce the complexity and/or dimensionality of the data set. For example, the use of multiple manipulations on a training data set can generate an N-dimensional space (e.g., probability plot) that can be used to represent a test parameter. Analysis of test samples using a substantially similar set of manipulations can be used to generate an N-dimensional point for each of the test samples. The complexity and/or dimensionality of a test subject data set sometimes is reduced to a single value or N-dimensional point that can be readily compared to the N-dimensional space generated from the training data.

In some embodiments, data processing includes cross-validation. Cross-validation, sometimes is referred to as rotation estimation. In some embodiments, a cross-validation approach is applied to assess how accurately a predictive model will perform in practice using a test sample. In some embodiments, one round of cross-validation comprises partitioning a sample of data into complementary subsets, performing a cross validation analysis on one subset (e.g., sometimes referred to as a training set), and validating the analysis using another subset (e.g., sometimes called a validation set or test set). In certain embodiments, multiple rounds of cross-validation are performed using different partitions and/or different subsets). Non-limiting examples of cross-validation approaches include leave-one-out, sliding edges, K-fold, 2-fold, repeat random sub-sampling, the like or combinations thereof. In some embodiments, a cross-validation randomly selects a work set containing 80% of a set of samples with known test parameters and uses that subset to train a model. In certain embodiments, the random selection is repeated multiple times.

### Statistical Models and Model Parameters

A method described herein may comprise assessing a feature of a test sample (e.g., presence or absence of a CNV; presence, absence, or degree of mosaicism; fetal fraction) according to one or more model parameters obtained from one or more models. A method described herein may comprise applying one or more model parameters from one or more models to one or more sequence tag quantifications for a test sample. A method described herein may comprise applying one or more model parameters from one or more models to a first test sequence tag quantification for fragments within a first selected fragment length range. A method described herein may comprise applying one or more model parameters from one or more models to a second test sequence tag quantification for fragments within a second selected fragment length range. A method described herein may comprise applying one or more model parameters from one or more models to i) a first test sequence tag quantification for fragments within a first selected fragment length range, and ii) a second test sequence tag quantification for fragments within a second selected fragment length range. A method described herein may comprise applying one or more model parameters from one or more models to a ratio of i) a first test sequence tag quantification for fragments within a first selected fragment length range to ii) a second test sequence tag quantification for fragments within a second selected fragment length range.

A model parameter may include values for one or more measured features described herein (e.g., (i) a first test sequence tag quantification for fragments within a first selected fragment length range; (ii) a second test sequence tag quantification for fragments within a second selected fragment length range; a ratio of (i) to (ii)) and a known or measured value (e.g., fetal fraction) for one or more samples (e.g., one or more samples in a training set). For example, values for one or more measured features described herein may be determined from aggregate values as determined for a sample (e.g., a training sample) for which fetal fraction is known. Model parameters may be defined in a variety of ways, for example as discrete values or as a model function (e.g., a model curve). A model function may be derived from one or more additional mathematical transformations of one or more model parameters.

In one example, a model parameter is a coefficient or constant that, in part, describes and/or defines a relation between fetal fraction and one or more measured features described herein (e.g., (i) a first test sequence tag quantification for fragments within a first selected fragment length range; (ii) a second test sequence tag quantification for fragments within a second selected fragment length range; a ratio of (i) to (ii))). In some embodiments, a model parameter is determined according to a relation for multiple fetal fractions and multiple measured features described herein. A relation may be defined by one or more model parameters and one or more model parameters may be determined from a relation. In some embodiments, a model parameter is determined from a fitted relation according to (i) a fraction of fetal nucleic acid for each of multiple samples, and (ii) one or more measured features for multiple samples.

A model parameter can be any suitable coefficient, estimated coefficient or constant derived from a suitable statistical model and/or relation (e.g., a suitable mathematical relation, an algebraic relation, a fitted relation, a regression, a regression analysis, a regression model). A model parameter can be determined according to, derived from, or estimated from a suitable relation. In some embodiments, a model parameter is an estimated coefficient from a fitted relation. Fitting a relation for multiple samples (e.g., multiple samples in a training set) is sometimes referred to herein as training a model. Any suitable model and/or method of fitting a relationship (e.g., training a model to a training set) can be used. Non-limiting examples of a suitable model that can be used include a regression model, linear regression model, simple regression model, ordinary least squares regression model, multiple regression model, general multiple regression model, polynomial regression model, general linear model, generalized linear model, discrete choice regression model, logistic regression model, multinomial logit model, mixed logit model, probit model, multinomial probit model, ordered logit model, ordered probit model, Poisson model, multivariate response regression model, multilevel model, fixed effects model, random effects model, mixed model, nonlinear regression model, nonparametric model, semiparametric model, robust model, quantile model, isotonic model, principal components model, least angle model, local model, segmented model, errors-in-variables model, and combinations thereof. In some embodiments, a fitted relation is not a regression model. In some embodiments, a fitted relation is chosen from a decision tree model, support-vector machine model, and neural network model. The result of training a model (e.g., a regression model, a relation) is often a relation that can be described mathematically where the relation comprises one or more coefficients (e.g., model parameters). For example, for a linear least squared model, a general multiple regression model can be trained using fetal fraction values and one or more measured features described herein resulting in a relationship. More complex multivariate models may determine one, two, three or more model parameters. In some embodiments, a model is trained according to fetal fraction and two or more measured features described herein obtained from multiple samples (e.g., fitted relationships fitted to multiple samples, e.g., by a matrix).

In some embodiments, a relationship is a geometric and/or graphical relationship. The terms “relationship” and “relation”, as used herein, are synonymous. In some embodiments, a relationship is a mathematical relationship. In some embodiments, a relationship is plotted. In some embodiments, a relationship is a linear relationship. In certain embodiments, a relationship is a non-linear relationship. In certain embodiments, a relationship is a regression (e.g., a regression line). A regression can be a linear regression or a non-linear regression. A relationship can be expressed by a mathematical equation. Often a relationship is defined, in part, by one or more constants and/or one or more variables. A relationship can be generated by a method known in the art. A relationship in two dimensions can be generated for one or more samples, in certain embodiments, and a variable probative of error, or possibly probative of error, can be selected for one or more of the dimensions. A relationship can be generated, for example, using graphing software known in the art that plots a graph using values of two or more variables provided by a user. A relationship can be fitted using a method known in the art (e.g., by performing a regression, a regression analysis, e.g., by a suitable regression program, e.g., software). Certain relationships can be fitted by linear regression, and the linear regression can generate a slope value and intercept value. Certain relationships sometimes are not linear and can be fitted by a non-linear function, such as a parabolic, hyperbolic or exponential function (e.g., a quadratic function), for example.

A model parameter can be derived from a suitable relation (e.g., a suitable mathematical relation, an algebraic relation, a fitted relation, a regression, a regression analysis, a regression model) by a suitable method. In some embodiments, fitted relations are fitted by an estimation, non-limiting examples of which include least squares, ordinary least squares, linear, partial, total, generalized, weighted, non-linear, iteratively reweighted, ridge regression, least absolute deviations, Bayesian, Bayesian multivariate, reduced-rank, LASSO, Weighted Rank Selection Criteria (WRSC), Rank Selection Criteria (RSC), an elastic net estimator (e.g., an elastic net regression) and combinations thereof.

Model parameters may be obtained from any suitable sample or group of samples. In some embodiments, model parameters are obtained from a training set of samples. In some embodiments, the fraction of fetal nucleic acid is known for each sample in a training set of samples. In some embodiments, a CNV is known for each sample in a training set of samples. In some embodiments, a mosaicism is known for each sample in a training set of samples. A training set of samples may include multiple reference samples. A training set of samples may include euploid samples, aneuploid samples, or a combination of euploid samples and aneuploid samples. A training set of samples may include female samples (from pregnant subjects carrying female fetuses), male samples (from pregnant subjects carrying male fetuses), or a combination of female samples and male samples.

In some embodiments, a model parameter is determined according to one or more samples (e.g., a training set of samples). In some embodiments, a model parameter is determined according to a relation for fetal fraction (e.g., sample-specific fetal fraction) for multiple samples and one or more measured features determined according to multiple samples. In some embodiments, a model parameter is determined according to a relation for CNV (e.g., sample-specific CNV) for multiple samples and one or more measured features determined according to multiple samples. In some embodiments, a model parameter is determined according to a relation for mosaicism (e.g., sample-specific mosaicism) for multiple samples and one or more measured features determined according to multiple samples. Model parameters are often determined from multiple samples, for example, from about 20 to about 100,000 or more, from about 100 to about 100,000 or more, from about 500 to about 100,000 or more, from about 1000 to about 100,000 or more, or from about 10,000 to about 100,000 or more samples. In some embodiments, model parameters are determined from about 1,000 samples to about 2,000 samples. Model parameters can be determined from samples that are euploid (e.g., samples from subjects bearing a euploid fetus, e.g., samples where no aneuploid chromosome is present). In some embodiments, model parameters are obtained from samples comprising an aneuploid chromosome (e.g., samples from subjects bearing an aneuploid fetus). In some embodiments, model parameters are determined from multiple samples from subjects bearing a euploid fetus and from subjects bearing a trisomy fetus. Model parameters can be derived from multiple samples where the samples are from subjects bearing a male fetus and/or a female fetus.

In one example, a fetal fraction estimate can be determined for a sample (e.g., a test sample) by applying one or more model parameters to one or more measured features for the test sample. Applying one or more model parameters can comprise adjusting, converting and/or transforming one or more measured features according to a model parameter by applying any suitable mathematical manipulation, non-limiting examples of which include multiplication, division, addition, subtraction, integration, symbolic computation, algebraic computation, algorithm, trigonometric or geometric function, transformation (e.g., a Fourier transform), the like or combinations thereof. Applying one or more model parameters can comprise adjusting, converting and/or transforming one or more measured features according to suitable mathematical model.

### CNV and Prenatal Diagnoses

Cell-free fetal DNA and RNA circulating in maternal blood can be used for the early non-invasive prenatal diagnosis (NIPD) of an increasing number of genetic conditions, both for pregnancy management and to aid reproductive decision-making. The presence of cell-free DNA circulating in the bloodstream has been known for over 50 years. More recently, presence of small amounts of circulating fetal DNA was discovered in the maternal bloodstream during pregnancy (Lo et al., Lancet 350:485-487 [1997]). Thought to originate from dying placental cells, cell-free fetal DNA (cfDNA) has been shown to comprise short fragments typically fewer than 200 bp in length Chan et al., Clin Chem 50:88-92 [2004]), which can be discerned as early as 4 weeks gestation (Illanes et al., Early Human Dev 83:563-566 [2007]), and known to be cleared from the maternal circulation within hours of delivery (Lo et al., Am J Hum Genet 64:218-224 [1999]). In addition to cfDNA, fragments of cell-free fetal RNA (cfRNA) can also be discerned in the maternal bloodstream, originating from genes that are transcribed in the fetus or placenta. The extraction and subsequent analysis of these fetal genetic elements from a maternal blood sample offers novel opportunities for NIPD.

In some embodiments, the aneuploidy is a complete chromosomal trisomy or monosomy, or a partial trisomy or monosomy. Partial aneuploidies are caused by loss or gain of part of a chromosome, and encompass chromosomal imbalances resulting from unbalanced translocations, unbalanced inversions, deletions and insertions. By far, the most commonly known aneuploidy compatible with life is trisomy 21, i.e., Down Syndrome (DS), which is caused by the presence of part or all of chromosome 21. Rarely, DS can be caused by an inherited or sporadic defect whereby an extra copy of all or part of chromosome 21 becomes attached to another chromosome (usually chromosome 14) to form a single aberrant chromosome. DS is associated with intellectual impairment, severe learning difficulties and excess mortality caused by long-term health problems such as heart disease. Other aneuploidies with known clinical significance include Edward syndrome (trisomy 18) and Patau Syndrome (trisomy 13), which are frequently fatal within the first few months of life. Abnormalities associated with the number of sex chromosomes are also known and include monosomy X, e.g., Turner syndrome (XO), and triple X syndrome (XXX) in female births and Kleinefelter syndrome (XXY) and XYY syndrome in male births, which are all associated with various phenotypes including sterility and reduction in intellectual skills. Monosomy X [45, X] is a common cause of early pregnancy loss accounting for about 7% of spontaneous abortions. Based on the liveborn frequency of 45,X (also called Turner syndrome) of 1-2/10,000, it is estimated that less than 1% of 45,X conceptions will survive to term. About 30% of Turners syndrome patients are mosaic with both a 45,X cell line and either a 46,XX cell line or one containing a rearranged X chromosome (Hook and Warburton 1983). The phenotype in a liveborn infant is relatively mild considering the high embryonic lethality and it has been hypothesized that possibly all liveborn females with Turner syndrome carry a cell line containing two sex chromosomes. Monosomy X can occur in females as 45,X or as 45,X/46XX, and in males as 45,X/46XY. Autosomal monosomies in human are generally suggested to be incompatible with life; however, there is quite a number of cytogenetic reports describing full monosomy of one chromosome 21 in live born children (Vosranova et al., Molecular Cytogen. 1:13 [2008]; Joosten et al., Prenatal Diagn. 17:271-5 [1997]. The method described herein can be used to diagnose these and other chromosomal abnormalities prenatally.

According to some embodiments, the methods disclosed herein can determine the presence or absence of chromosomal trisomies of any one of chromosomes 1-22, X and Y. Examples of chromosomal trisomies that can be detected according to the present method include without limitation trisomy 21 (T21; Down Syndrome), trisomy 18 (T18; Edward's Syndrome), trisomy 16 (T16), trisomy 20 (T20), trisomy 22 (T22; Cat Eye Syndrome), trisomy 15 (T15; Prader Willi Syndrome), trisomy 13 (T13; Patau Syndrome), trisomy 8 (T8; Warkany Syndrome), trisomy 9, and the XXY (Kleinefelter Syndrome), XYY, or XXX trisomies. Complete trisomies of other autosomes existing in a non-mosaic state are lethal, but can be compatible with life when present in a mosaic state. It will be appreciated that various complete trisomies, whether existing in a mosaic or non-mosaic state, and partial trisomies can be determined in fetal cfDNA according to the teachings provided herein.

Non-limiting examples of partial trisomies that can be determined by the present method include, but are not limited to, partial trisomy 1q32-44, trisomy 9 p, trisomy 4 mosaicism, trisomy 17p, partial trisomy 4q26-qter, partial 2p trisomy, partial trisomy 1q, and/or partial trisomy 6p/monosomy 6q.

The methods disclosed herein can be also used to determine chromosomal monosomy X, chromosomal monosomy 21, and partial monosomies such as, monosomy 13, monosomy 15, monosomy 16, monosomy 21, and monosomy 22, which are known to be involved in pregnancy miscarriage. Partial monosomy of chromosomes typically involved in complete aneuploidy can also be determined by the method described herein. Non-limiting examples of deletion syndromes that can be determined according to the present method include syndromes caused by partial deletions of chromosomes. Examples of partial deletions that can be determined according to the methods described herein include without limitation partial deletions of chromosomes 1, 4, 5, 7, 11, 18, 15, 13, 17, 22 and 10, which are described in the following.

1q21.1 deletion syndrome or 1q21.1 (recurrent) microdeletion is a rare aberration of chromosome 1. Next to the deletion syndrome, there is also a 1q21.1 duplication syndrome. While there is a part of the DNA missing with the deletion syndrome on a particular spot, there are two or three copies of a similar part of the DNA on the same spot with the duplication syndrome. Literature refers to both the deletion and the duplication as the 1q21.1 copy-number variations (CNV). The 1q21.1 deletion can be associated with the TAR Syndrome (Thrombocytopenia with Absent radius).

Wolf-Hirschhorn syndrome (WHS) (OMIN #194190) is a contiguous gene deletion syndrome associated with a hemizygous deletion of chromosome 4p16.3. Wolf-Hirschhorn syndrome is a congenital malformation syndrome characterized by pre- and postnatal growth deficiency, developmental disability of variable degree, characteristic craniofacial features (‘Greek warrior helmet’ appearance of the nose, high forehead, prominent glabella, hypertelorism, high-arched eyebrows, protruding eyes, epicanthal folds, short philtrum, distinct mouth with downturned corners, and micrognathia), and a seizure disorder.

Partial deletion of chromosome 5, also known as 5p- or 5p minus, and named Cris du Chat syndrome (OMIN #123450), is caused by a deletion of the short arm (p arm) of chromosome 5 (5p15.3-p15.2). Infants with this condition often have a high-pitched cry that sounds like that of a cat. The disorder is characterized by intellectual disability and delayed development, small head size (microcephaly), low birth weight, and weak muscle tone (hypotonia) in infancy, distinctive facial features and possibly heart defects.

Williams-Beuren Syndrome also known as chromosome 7q11.23 deletion syndrome (OMIN 194050) is a contiguous gene deletion syndrome resulting in a multisystem disorder caused by hemizygous deletion of 1.5 to 1.8 Mb on chromosome 7q11.23, which contains approximately 28 genes.

Jacobsen Syndrome, also known as 11q deletion disorder, is a rare congenital disorder resulting from deletion of a terminal region of chromosome 11 that includes band 11q24.1. It can cause intellectual disabilities, a distinctive facial appearance, and a variety of physical problems including heart defects and a bleeding disorder.

Partial monosomy of chromosome 18, known as monosomy 18p is a rare chromosomal disorder in which all or part of the short arm (p) of chromosome 18 is deleted (monosomic). The disorder is typically characterized by short stature, variable degrees of mental retardation, speech delays, malformations of the skull and facial (craniofacial) region, and/or additional physical abnormalities. Associated craniofacial defects may vary greatly in range and severity from case to case.

Conditions caused by changes in the structure or number of copies of chromosome 15 include Angelman Syndrome and Prader-Willi Syndrome, which involve a loss of gene activity in the same part of chromosome 15, the 15q11-q13 region. It will be appreciated that several translocations and microdeletions can be asymptomatic in the carrier parent, yet can cause a major genetic disease in the offspring. For example, a healthy mother who carries the 15q11-q13 microdeletion can give birth to a child with Angelman syndrome, a severe neurodegenerative disorder. Thus, the methods, apparatus and systems described herein can be used to identify such a partial deletion and other deletions in the fetus.

Partial monosomy 13q is a rare chromosomal disorder that results when a piece of the long arm (q) of chromosome 13 is missing (monosomic). Infants born with partial monosomy 13q may exhibit low birth weight, malformations of the head and face (craniofacial region), skeletal abnormalities (especially of the hands and feet), and other physical abnormalities. Mental retardation is characteristic of this condition. The mortality rate during infancy is high among individuals born with this disorder. Almost all cases of partial monosomy 13q occur randomly for no apparent reason (sporadic).

Smith-Magenis syndrome (SMS-OMIM #182290) is caused by a deletion, or loss of genetic material, on one copy of chromosome 17. This well-known syndrome is associated with developmental delay, mental retardation, congenital anomalies such as heart and kidney defects, and neurobehavioral abnormalities such as severe sleep disturbances and self-injurious behavior. Smith-Magenis syndrome (SMS) is caused in most cases (90%) by a 3.7-Mb interstitial deletion in chromosome 17p11.2.

22q11.2 deletion syndrome, also known as DiGeorge syndrome, is a syndrome caused by the deletion of a small piece of chromosome 22. The deletion (22 q11.2) occurs near the middle of the chromosome on the long arm of one of the pair of chromosomes. The features of this syndrome vary widely, even among members of the same family, and affect many parts of the body. Characteristic signs and symptoms may include birth defects such as congenital heart disease, defects in the palate, most commonly related to neuromuscular problems with closure (velo-pharyngeal insufficiency), learning disabilities, mild differences in facial features, and recurrent infections. Microdeletions in chromosomal region 22q11.2 are associated with a 20 to 30-fold increased risk of schizophrenia.

Deletions on the short arm of chromosome 10 are associated with a DiGeorge Syndrome like phenotype. Partial monosomy of chromosome 10p is rare but has been observed in a portion of patients showing features of the DiGeorge Syndrome.

In one embodiment, the methods, apparatus, and systems described herein is used to determine partial monosomies including but not limited to partial monosomy of chromosomes 1, 4, 5, 7, 11, 18, 15, 13, 17, 22 and 10, e.g., partial monosomy 1q21.11, partial monosomy 4p16.3, partial monosomy 5p15.3-p15.2, partial monosomy 7q11.23, partial monosomy 11q24.1, partial monosomy 18p, partial monosomy of chromosome 15 (15q11-q13), partial monosomy 13q, partial monosomy 17p11.2, partial monosomy of chromosome 22 (22q11.2), and partial monosomy 10p can also be determined using the method.

Other partial monosomies that can be determined according to the methods described herein include unbalanced translocation t(8;11)(p23.2;p15.5); 11q23 microdeletion; 17p11.2 deletion; 22q13.3 deletion; Xp22.3 microdeletion; 10p14 deletion; 20p microdeletion, [del(22)(q11.2q11.23)], 7q11.23 and 7q36 deletions; 1p36 deletion; 2p microdeletion; neurofibromatosis type 1 (17q11.2 microdeletion), Yq deletion; 4p16.3 microdeletion; 1p36.2 microdeletion; 11q14 deletion; 19q13.2 microdeletion; Rubinstein-Taybi (16 p13.3 microdeletion); 7p21 microdeletion; Miller-Dieker syndrome (17p13.3); and 2q37 microdeletion. Partial deletions can be small deletions of part of a chromosome, or they can be microdeletions of a chromosome where the deletion of a single gene can occur.

Several duplication syndromes caused by the duplication of part of chromosome arms have been identified (see OMIN [Online Mendelian Inheritance in Man viewed online at ncbi.nlm.nih.gov/omim]). In one embodiment, the present method can be used to determine the presence or absence of duplications and/or multiplications of segments of any one of chromosomes 1-22, X and Y. Non-limiting examples of duplications syndromes that can be determined according to the present method include duplications of part of chromosomes 8, 15, 12, and 17, which are described in the following.

8p23.1 duplication syndrome is a rare genetic disorder caused by a duplication of a region from human chromosome 8. This duplication syndrome has an estimated prevalence of 1 in 64,000 births and is the reciprocal of the 8p23.1 deletion syndrome. The 8p23.1 duplication is associated with a variable phenotype including one or more of speech delay, developmental delay, mild dysmorphism, with prominent forehead and arched eyebrows, and congenital heart disease (CHD).

Chromosome 15q Duplication Syndrome (Dup15q) is a clinically identifiable syndrome which results from duplications of chromosome 15q11-13.1 Babies with Dup15q usually have hypotonia (poor muscle tone), growth retardation; they may be born with a cleft lip and/or palate or malformations of the heart, kidneys or other organs; they show some degree of cognitive delay/disability (mental retardation), speech and language delays, and sensory processing disorders.

Pallister Killian syndrome is a result of extra #12 chromosome material. There is usually a mixture of cells (mosaicism), some with extra #12 material, and some that are normal (46 chromosomes without the extra #12 material). Babies with this syndrome have many problems including severe mental retardation, poor muscle tone, “coarse” facial features, and a prominent forehead. They tend to have a very thin upper lip with a thicker lower lip and a short nose. Other health problems include seizures, poor feeding, stiff joints, cataracts in adulthood, hearing loss, and heart defects. Persons with Pallister Killian have a shortened lifespan.

Individuals with the genetic condition designated as dup(17)(p11.2p11.2) or dup 17p carry extra genetic information (known as a duplication) on the short arm of chromosome 17. Duplication of chromosome 17p11.2 underlies Potocki-Lupski syndrome (PTLS), which is a newly recognized genetic condition with only a few dozen cases reported in the medical literature. Patients who have this duplication often have low muscle tone, poor feeding, and failure to thrive during infancy, and also present with delayed development of motor and verbal milestones. Many individuals who have PTLS have difficulty with articulation and language processing. In addition, patients may have behavioral characteristics similar to those seen in persons with autism or autism-spectrum disorders. Individuals with PTLS may have heart defects and sleep apnea. A duplication of a large region in chromosome 17p12 that includes the gene PMP22 is known to cause Charcot-Marie Tooth disease.

CNVs have been associated with stillbirths. However, due to inherent limitations of conventional cytogenetics, the contribution of CNVs to stillbirth is thought to be underrepresented (Harris et al., Prenatal Diagn 31:932-944 [2011]).

### Determination of CNV of Clinical Disorders

In addition to the early determination of birth defects, the methods described herein can be applied to the determination of any abnormality in the representation of genetic sequences within the genome. A number of abnormalities in the representation of genetic sequences within the genome have been associated with various pathologies. Such pathologies include, but are not limited to cancer, infectious and autoimmune diseases, diseases of the nervous system, metabolic and/or cardiovascular diseases, and the like.

Accordingly in various embodiments, use of the methods described herein in the diagnosis, and/or monitoring, and or treating such pathologies is contemplated. For example, the methods can be applied to determining the presence or absence of a disease, to monitoring the progression of a disease and/or the efficacy of a treatment regimen, to determining the presence or absence of nucleic acids of a pathogen e.g., virus; to determining chromosomal abnormalities associated with graft versus host disease (GVHD), and to determining the contribution of individuals in forensic analyses.

### CNVs in Cancer

It has been shown that blood plasma and serum DNA from cancer patients contains measurable quantities of tumor DNA, that can be recovered and used as surrogate source of tumor DNA, and tumors are characterized by aneuploidy, or inappropriate numbers of gene sequences or even entire chromosomes. The determination of a difference in the amount of a given sequence i.e., a sequence of interest, in a sample from an individual can thus be used in the prognosis or diagnosis of a medical condition. In some embodiments, the present method can be used to determine the presence or absence of a chromosomal aneuploidy in a patient suspected or known to be suffering from cancer.

Some implementations herein provide methods for detecting cancer, tracking therapeutic response and minimal residual disease based on circulating cfDNA samples using shallow sequencing of the samples with paired-end methodology and using fragment size information available from paired-end reads. It has been shown that tumor-derived cfDNA are shorter than non-tumor-derived cfDNA in some cancers. Therefore, the size-based method described herein can be used to determine CNVs including aneuploidies associated with these cancers, enabling (a) detection of tumor present in a screening or diagnostic setting; (b) monitoring response to therapy; (c) monitoring minimal residual disease.

In certain embodiments, the aneuploidy is characteristic of the genome of the subject and results in a generally increased predisposition to a cancer. In certain embodiments, the aneuploidy is characteristic of particular cells (e.g., tumor cells, proto-tumor neoplastic cells, and the like) that are or have an increased predisposition to neoplasia. Particular aneuploidies are associated with particular cancers or predispositions to particular cancers as described below. In some embodiments, a very shallow paired-end sequencing approach can be used to detect/monitor cancer presence in a cost-effective way.

Accordingly, various embodiments of the methods described herein provide a determination of copy number variation of sequence(s) of interest e.g., clinically-relevant sequence(s), in a test sample from a subject where certain variations in copy number provide an indicator of the presence and/or a predisposition to a cancer. In certain embodiments, the sample comprises a mixture of nucleic acids is derived from two or more types of cells. In one embodiment, the mixture of nucleic acids is derived from normal and cancerous cells derived from a subject suffering from a medical condition e.g., cancer.

The development of cancer is often accompanied by an alteration in number of whole chromosomes i.e., complete chromosomal aneuploidy, and/or an alteration in the number of segments of chromosomes i.e., partial aneuploidy, caused by a process known as chromosome instability (CIN) (Thoma et al., Swiss Med Weekly 2011:141:w13170). It is believed that many solid tumors, such as breast cancer, progress from initiation to metastasis through the accumulation of several genetic aberrations. [Sato et al., Cancer Res., 50: 7184-7189 [1990]; Jongsma et al., J Clin Pathol: Mol Path 55:305-309 [2002])]. Such genetic aberrations, as they accumulate, may confer proliferative advantages, genetic instability and the attendant ability to evolve drug resistance rapidly, and enhanced angiogenesis, proteolysis and metastasis. The genetic aberrations may affect either recessive “tumor suppressor genes” or dominantly acting oncogenes. Deletions and recombination leading to loss of heterozygosity (LOH) are believed to play a major role in tumor progression by uncovering mutated tumor suppressor alleles.

cfDNA has been found in the circulation of patients diagnosed with malignancies including but not limited to lung cancer (Pathak et al. Clin Chem 52:1833-1842 [2006]), prostate cancer (Schwartzenbach et al. Clin Cancer Res 15:1032-8 [2009]), and breast cancer (Schwartzenbach et al. available online at breast-cancer-research.com/content/11/5/R71 [2009]). Identification of genomic instabilities associated with cancers that can be determined in the circulating cfDNA in cancer patients is a potential diagnostic and prognostic tool. In one embodiment, methods described herein are used to determine CNV of one or more sequence(s) of interest in a sample, e.g., a sample comprising a mixture of nucleic acids derived from a subject that is suspected or is known to have cancer e.g., carcinoma, sarcoma, lymphoma, leukemia, germ cell tumors and blastoma. In one embodiment, the sample is a plasma sample derived (processed) from peripheral blood that may comprise a mixture of cfDNA derived from normal and cancerous cells. In another embodiment, the biological sample that is needed to determine whether a CNV is present is derived from a cells that, if a cancer is present, comprise a mixture of cancerous and non-cancerous cells from other biological tissues including, but not limited to biological fluids such as serum, sweat, tears, sputum, urine, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, and leukophoresis samples, or in tissue biopsies, swabs, or smears. In other embodiments, the biological sample is a stool (fecal) sample.

The methods described herein are not limited to the analysis of cfDNA. It will be recognized that similar analyses can be performed on cellular DNA samples.

In various embodiments, the sequence(s) of interest comprise nucleic acid sequence(s) known or is suspected to play a role in the development and/or progression of the cancer. Examples of a sequence of interest include nucleic acids sequences e.g., complete chromosomes and/or segments of chromosomes, that are amplified or deleted in cancerous cells as described below.

### Total CNV Number and Risk for Cancer

Common cancer SNPs—and by analogy common cancer CNVs may each confer only a minor increase in disease risk. However, collectively they may cause a substantially elevated risk for cancers. In this regard it is noted that germline gains and losses of large DNA segments have been reported as factors predisposing individuals to neuroblastoma, prostate and colorectal cancer, breast cancer, and BRCA1-associated ovarian cancer (see, e.g., Krepischi et al. Breast Cancer Res., 14: R24 [2012]; Diskin et al. Nature 2009, 459:987-991; Liu et al. Cancer Res 2009, 69: 2176-2179; Lucito et al. Cancer Biol Ther 2007, 6:1592-1599; Thean et al. Genes Chromosomes Cancer 2010, 49:99-106; Venkatachalam et al. Int J Cancer 2011, 129:1635-1642; and Yoshihara et al. Genes Chromosomes Cancer 2011, 50:167-177). It is noted that CNVs frequently found in the healthy population (common CNVs) are believed to have a role in cancer etiology (see, e.g., Shlien and Malkin (2009) Genome Medicine, 1(6): 62). In one study testing the hypothesis that common CNVs are associated with malignancy (Shlien et al. Proc Natl Acad Sci USA 2008, 105:11264-11269) a map of every known CNV whose locus coincides with that of bona fide cancer-related genes (as catalogued by Higgins et al. Nucleic Acids Res 2007, 35:D721-726) was created. These were termed “cancer CNVs”. In an initial analysis (Shlien et al. Proc Natl Acad Sci USA 2008, 105:11264-11269), 770 healthy genomes were evaluated using the Affymetrix 500K array set, which has an average inter-probe distance of 5.8 kb. As CNVs are generally thought to be depleted in gene regions (Redon et al. (2006) Nature 2006, 444:444-454), it was surprising to find 49 cancer genes that were directly encompassed or overlapped by a CNV in more than one person in a large reference population. In the top ten genes, cancer CNVs could be found in four or more people.

It is thus believed that CNV frequency can be used as a measure of risk for cancer (see, e.g., U.S. Patent Publication No: 2010/0261183 A1). The CNV frequency can be determined simply by the constitutive genome of the organism or it can represent a fraction derived from one or more tumors (neoplastic cells) if such are present.

In certain embodiments, a number of CNVs in a test sample (e.g., a sample comprising a constitutional (germline) nucleic acid) or a mixture of nucleic acids (e.g., a germline nucleic acid and nucleic acid(s) derived from neoplastic cells) is determined using the methods described herein for copy number variations. Identification of an increased number of CNVs in the test sample, e.g., in comparison to a reference value is indicative of a risk of or pre-disposition for cancer in the subject. It will be appreciated that the reference value may vary with a given population. It will also be appreciated that the absolute value of the increase in CNV frequency will vary depending on the resolution of the method utilized to determine CNV frequency and other parameters. Typically, an increase in CNV frequency of at least about 1.2 times the reference value been determined to indicative of risk for cancer (see, e.g., U.S. Patent Publication No: 2010/0261183 A1), for example an increase in CNV frequency of at least or about 1.5 times the reference value or greater, such as 2-4 times the reference value is an indicator of an increased risk of cancer (e.g., as compared to the normal healthy reference population).

A determination of structural variation in the genome of a mammal in comparison to a reference value is also believed to be indicative of risk of cancer. In this context, in one embodiment, the term “structural variation” is can be defined as the CNV frequency in a mammal multiplied by the average CNV size (in bp) in the mammal. Thus, high structural variation scores will result due to increased CNV frequency and/or due to the occurrence of large genomic nucleic acid deletions or duplications. Accordingly, in certain embodiments, a number of CNVs in a test sample (e.g., a sample comprising a constitutional (germline) nucleic acid) is determined using the methods described herein to determine size and number of copy number variations. In certain embodiments, a total structural variation score within genomic DNA of greater than about 1 megabase, or greater than about 1.1 megabases, or greater than about 1.2 megabases, or greater than about 1.3 megabases, or greater than about 1.4 megabases, or greater than about 1.5 megabases, or greater than about 1.8 megabases, or greater than about 2 megabases of DNA is indicative of risk of cancer.

It is believed these methods provide a measure of the risk of any cancer including but not limited to, acute and chronic leukemias, lymphomas, numerous solid tumors of mesenchymal or epithelial tissue, brain, breast, liver, stomach, colon cancer, B cell lymphoma, lung cancer, a bronchus cancer, a colorectal cancer, a prostate cancer, a breast cancer, a pancreas cancer, a stomach cancer, an ovarian cancer, a urinary bladder cancer, a brain or central nervous system cancer, a peripheral nervous system cancer, an esophageal cancer, a cervical cancer, a melanoma, a uterine or endometrial cancer, a cancer of the oral cavity or pharynx, a liver cancer, a kidney cancer, a biliary tract cancer, a small bowel or appendix cancer, a salivary gland cancer, a thyroid gland cancer, a adrenal gland cancer, an osteosarcoma, a chondrosarcoma, a liposarcoma, a testes cancer, and a malignant fibrous histiocytoma, and other cancers.

### Full Chromosome Aneuploidies.

As indicated above, there exists a high frequency of aneuploidy in cancer. In certain studies, examining the prevalence of somatic copy number alterations (SCNAs) in cancer, it has been discovered that one-quarter of the genome of a typical cancer cell is affected either by whole-arm SCNAs or by the whole-chromosome SCNAs of aneuploidy (see, e.g., Beroukhim et al. Nature 463: 899-905 [2010]). Whole-chromosome alterations are recurrently observed in several cancer types. For example, the gain of chromosome 8 is seen in 10-20% of cases of acute myeloid leukaemia (AML), as well as some solid tumours, including Ewing's Sarcoma and desmoid tumours (see, e.g., Barnard et al. Leukemia 10: 5-12 [1996]; Maurici et al. Cancer Genet. Cytogenet. 100: 106-110 [1998]; Qi et al. Cancer Genet. Cytogenet. 92: 147-149 [1996]; Barnard, D. R. et al. Blood 100: 427-434 [2002]; and the like. Illustrative, but non-limiting list of chromosome gains and losses in human cancers are shown in Table 1.

TABLE 1
Illustrative specific, recurrent chromosome gains
and losses in human cancer (see, e.g., Gordon et al. (2012)
Nature Rev. Genetics, 13: 189-203).

| | Gains | Losses |
|---|---|---|
| Chromosome | Cancer Type | Cancer Type |
| 1 | Multiple myeloma | Adenocarcinoma |
| | Adenocarcinoma (breast) | (kidney) |
| 2 | Hepatoblastoma | |
| | Ewing's sarcoma | |
| 3 | Multiple myeloma | Melanoma |
| | Diffuse large B-cell lymphoma | Adenocarcinoma |
| | | (kidney) |
| 4 | Acute lymphoblastic leukaemia | Adenocarcinoma |
| 5 | Multiple myeloma | |
| | Adenocarcinoma (kidney) | (kidney) |
| 6 | Acute lymphoblastic leukaemia | Adenocarcinoma |
| | Wilms' tumour | (kidney) |
| 7 | Adenocarcinoma (kidney) | Acute myeloid |
| | Adenocarcinoma (intestine) | leukaemia |
| | | Juvenile |
| | | myelomonocytic |
| | | leukaemia |
| 8 | Acute myeloid leukaemia | Adenocarcinoma |
| | Chronic myeloid leukaemia | (kidney) |
| | Ewing's sarcoma | |
| 9 | Multiple myeloma | |
| | Polycythaemia vera | |
| 10 | Acute lymphoblastic leukaemia | Astrocytoma |
| | Adenocarcinoma (uterus) | Multiple myeloma |
| 11 | Multiple myeloma | |
| 12 | Chronic lymphocytic leukaemia | Multiple myeloma |
| | Wilms' tumor | |
| 13 | Acute myeloid leukaemia | Multiple myeloma |
| | Wilms' tumor | |
| 14 | Acute lymphoblastic leukaemia | Adenocarcinoma |
| | | (kidney) |
| | | Meningioma |
| 15 | Multiple myeloma | |
| 16 | Adenocarcinoma (kidney) | Multiple myeloma |
| 17 | Adenocarcinoma (kidney) | |
| | Acute lymphoblastic leukaemia | |
| 18 | Acute lymphoblastic leukaemia | Adenocarcinoma |
| | Wilms' tumour | (kidney) |
| 19 | Multiple myeloma | Adenocarcinoma |
| | Chronic myeloid leukaemia | (Breast) |
| | | Meningioma |
| 20 | Hepatoblastoma | |
| | Adenocarcinoma (kidney) | |
| 21 | Acute lymphoblastic leukaemia | |
| | Acute megakaryoblastic leukaemia | |
| 22 | Acute lymphoblastic leukaemia | Meningioma |
| X | Acute lymphoblastic leukaemia | |
| | Follicular lymphoma | |
| Y | | |

In various embodiments, the methods described herein can be used to detect and/or quantify whole chromosome aneuploidies that are associated with cancer generally, and/or that are associated with particular cancers. Thus, for example, in certain embodiments, detection and/or quantification of whole chromosome aneuploidies characterized by the gains or losses shown in Table 1 are contemplated.

### Arm Level Chromosomal Segment Copy Number Variations

Multiple studies have reported patterns of arm-level copy number variations across large numbers of cancer specimens (Lin et al. Cancer Res 68, 664-673 (2008); George et al. PLoS ONE 2, e255 (2007); Demichelis et al. Genes Chromosomes Cancer 48: 366-380 (2009); Beroukhim et al. Nature. 463(7283): 899-905 [2010]). It has additionally been observed that the frequency of arm-level copy number variations decreases with the length of chromosome arms. Adjusted for this trend, the majority of chromosome arms exhibit strong evidence of preferential gain or loss, but rarely both, across multiple cancer lineages (see, e.g., Beroukhim et al. Nature. 463(7283): 899-905 [2010]).

Accordingly, in one embodiment, methods described herein are used to determine arm level CNVs (CNVs comprising one chromosomal arm or substantially one chromosomal arm) in a sample. The CNVs can be determined in a CNVs in a test sample comprising a constitutional (germline) nucleic acid and the arm level CNVs can be identified in those constitutional nucleic acids. In certain embodiments, arm level CNVs are identified (if present) in a sample comprising a mixture of nucleic acids (e.g., nucleic acids derived from normal and nucleic acids derived from neoplastic cells). In certain embodiments, the sample is derived from a subject that is suspected or is known to have cancer e.g., carcinoma, sarcoma, lymphoma, leukemia, germ cell tumors, blastoma, and the like. In one embodiment, the sample is a plasma sample derived (processed) from peripheral blood that may comprise a mixture of cfDNA derived from normal and cancerous cells. In another embodiment, the biological sample that is used to determine whether a CNV is present is derived from a cells that, if a cancer is present, comprise a mixture of cancerous and non-cancerous cells from other biological tissues including, but not limited to biological fluids such as serum, sweat, tears, sputum, urine, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, and leukophoresis samples, or in tissue biopsies, swabs, or smears. In other embodiments, the biological sample is a stool (fecal) sample.

In various embodiments, the CNVs identified as indicative of the presence of a cancer or an increased risk for a cancer include, but are not limited to the arm level CNVs listed in Table 2. As illustrated in Table 2 certain CNVs that comprise a substantial arm-level gain are indicative of the presence of a cancer or an increased risk for certain cancers. Thus, for example, a gain in 1q is indicative of the presence or increased risk for acute lymphoblastic leukemia (ALL), breast cancer, GIST, HCC, lung NSC, medulloblastoma, melanoma, MPD, ovarian cancer, and/or prostate cancer. A gain in 3q is indicative of the presence or increased risk for Esophageal Squamous cancer, Lung SC, and/or MPD. A gain in 7q is indicative of the presence or increased risk for colorectal cancer, glioma, HCC, lung NSC, medulloblastoma, melanoma, prostate cancer, and/or renal cancer. A gain in 7p is indicative of the presence or increased risk for breast cancer, colorectal cancer, esophageal adenocarcinoma, glioma, HCC, Lung NSC, medulloblastoma, melanoma, and/or renal cancer. A gain in 20q is indicative of the presence or increased risk for breast cancer, colorectal cancer, dedifferentiated liposarcoma, esophageal adenocarcinoma, esophageal squamous, glioma cancer, HCC, lung NSC, melanoma, ovarian cancer, and/or renal cancer, and so forth.

Similarly, as illustrated in Table 2 certain CNVs that comprise a substantial arm-level loss are indicative of the presence of and/or an increased risk for certain cancers. Thus, for example, a loss in 1 p is indicative of the presence or increased risk for gastrointestinal stromal tumor. A loss in 4q is indicative of the presence or increased risk for colorectal cancer, esophageal adenocarcinoma, lung sc, melanoma, ovarian cancer, and/or renal cancer. A loss in 17p is indicative of the presence or increased risk for breast cancer, colorectal cancer, esophageal adenocarcinoma, HCC, lung NSC, lung SC, and/or ovarian cancer, and the like.

TABLE 2
Significant arm-level chromosomal segment copy number alterations
in each of 16 cancer subtypes (breast, colorectal, dedifferentiated
liposarcoma, esphageal adenocarcinoma, esophageal squamous,
GIST (gastrointestinal stromal tumor), glioma, HCC (hepatocellular
carcinoma), lung NSC, lung SC, medulloblastoma, melanoma, MPD
(myeloproliferative disease), ovarian, prostate, acute lymphoblastic leukemia (ALL),
and renal) (see, e.g., Beroukhim et al. Nature (2010) 463(7283): 899-905)

| | Cancer Types | Cancer Types | Known |
|---|---|---|---|
| | Significantly | Significantly | Oncogene/Tumor |
| Arm | Gained In | Lost In | Suppressor Gene |
| 1p | — | GIST | |
| 1q | ALL, Breast, GIST, | — | |
| | HCC, Lung NSC, | | |
| | Medullablastoma, | | |
| | Melanoma, MPD, | | |
| | Ovarian, Prostate | | |
| 3p | — | Esophageal Squamous, | VHL |
| | | Lung NSC, Lung SC, Renal | |
| 3q | Esophageal Squamous, | — | |
| | Lung SC, MPD | | |
| 4p | ALL | Breast, Esophageal | |
| | | Adenocarcinoma, Renal | |
| 4q | ALL | Colorectal, Esophageal | |
| | | Adenocarcinoma, Lung | |
| | | SC, Melanoma, Ovarian, | |
| | | Renal | |
| 5p | Esophageal Squamous, | — | TERT |
| | HCC, Lung NSC, Lung | | |
| | SC, Renal | | |
| 5q | HCC, Renal | Esophageal Adenocarcinoma, | APC |
| | | Lung NSC | |
| 6p | ALL, HCC, Lung NSC, | — | |
| | Melanoma | | |
| 6q | ALL | Melanoma, Renal | |
| 7p | Breast, Colorectal, | — | EGFR |
| | Esophageal | | |
| | Adenocarcinoma, | | |
| | Glioma, HCC, Lung | | |
| | NSC, Medullablastoma, | | |
| | Melanoma, Renal | | |
| 7q | Colorectal, Glimoa, | — | BRAF, MET |
| | HCC, Lung NSC, | | |
| | Medulloblastoma, | | |
| | Melanoma, Prostate, | | |
| | Renal | | |
| 8p | ALL, MPD | Breast, HCC, Lung NSC, | |
| | | Medulloblatoma, Prostate, | |
| | | Renal | |
| 8q | ALL, Breast, Colorectal, | Medulloblastoma | MYC |
| | Esophageal | | |
| | Adenocarcinoma, | | |
| | Esophageal Squamous, | | |
| | HCC, Lung NSC, MPD, | | |
| | Ovarian, Prostate | | |
| 9p | MPD | ALL, Breast, Esophageal, | CDKN2A/B |
| | | Adenocarcinoma, Lung NSC, | |
| | | Melanoma, Ovarian, Renal | |
| 9q | ALL, MPD | Lung NSC, Melanoma, | |
| | | Ovarian, Renal | |
| 10p | ALL | Glioma, Lung SC, Melanoma | |
| 10q | ALL | Glioma, Lung SC, | |
| | | Medulloblastoma, Melanoma | |
| 11p | — | Medulloblastoma | WT1 |
| 11q | — | Dedifferentiated Liposarcoma, | ATM |
| | | Medulloblastoma, Melanoma | |
| 12p | Colorectal, Renal | — | KRAS |
| 12q | Renal | — | |
| 13q | Colorectal | Breast, Dedifferentiated | RB1/BRCA2 |
| | | Liposarcoma, Glioma, Lung | |
| | | NSC, Ovarian | |
| 14q | ALL, Lung NSC, Lung | GIST, Melanoma, Renal | |
| | SC, Prostate | | |
| 15q | — | GIST, Lung NSC, Lung SC, | |
| | | Ovarian | |
| 16p | Breast | — | |
| 16q | — | Breast, HCC, Medulloblastoma, | |
| | | Ovarian, Prostate | |
| 17p | ALL | Breast, Colorectal, Esophageal | TP53 |
| | | Adenocarcinoma, HCC, Lung | |
| | | NSC, Lung SC, Ovarian | |
| 17q | ALL, HCC, Lung NSC, | Breast, Ovarian | ERBB2, NF1/BRCA1 |
| | Medulloblastoma | | |
| 18p | ALL, Medulloblastoma | Colorectal, Lung NSC | |
| 18q | ALL, Medulloblastoma | Colorectal, Esophageal | SMAD2, SMAD4 |
| | | Adenocarcinoma, Lung NSC | |
| 19p | Glioma | Esophageal Adenocarcinoma, | |
| | | Lung NSC, Melanoma, Ovarian | |
| 19q | Glioma, Lung SC | Esophageal Adenocarcinoma, | |
| | | Lung NSC | |
| 20p | Breast, Colorectal, | — | |
| | Esophageal Adenocarcinoma, | | |
| | Esophageal Squamous, GIST, | | |
| | Glioma, HCC, Lung NSC, | | |
| | Melanoma, Renal | | |
| 20q | Breast, Colorectal, | — | |
| | Defifferentiated Liposarcoma, | | |
| | Esophageal Adenocarcinoma, | | |
| | Esophageal Squamous, | | |
| | Glioma, HCC, Lung NSC, | | |
| | Melanoma, Ovarian, Renal | | |
| 21q | ALL, GIST, MPD | — | |
| 22q | Melanoma | Breast, Colorectal, | NF2 |
| | | Dedifferentiated Liposarcoma, | |
| | | Esophageal Adenocarcinoma, | |
| | | GIST, Lung NSC, Lung SC, | |
| | | Ovarian, Prostate | |

The examples of associations between arm level copy number variations are intended to be illustrative and not limiting. Other arm level copy number variations and their cancer associations are known to those of skill in the art.

### Smaller, e.g., Focal, Copy Number Variations

As indicated above, in certain embodiments, the methods described herein can be used to determine the presence or absence of a chromosomal amplification. In some embodiments, the chromosomal amplification is the gain of one or more entire chromosomes. In other embodiments, the chromosomal amplification is the gain of one or more segments of a chromosome. In yet other embodiments, the chromosomal amplification is the gain of two or more segments of two or more chromosomes. In various embodiments, the chromosomal amplification can involve the gain of one or more oncogenes.

Dominantly acting genes associated with human solid tumors typically exert their effect by overexpression or altered expression. Gene amplification is a common mechanism leading to upregulation of gene expression. Evidence from cytogenetic studies indicates that significant amplification occurs in over 50% of human breast cancers. Most notably, the amplification of the proto-oncogene human epidermal growth factor receptor 2 (HER2) located on chromosome 17 (17(17q21-q22)), results in overexpression of HER2 receptors on the cell surface leading to excessive and dysregulated signaling in breast cancer and other malignancies (Park et al., Clinical Breast Cancer 8:392-401 [2008]). A variety of oncogenes have been found to be amplified in other human malignancies. Examples of the amplification of cellular oncogenes in human tumors include amplifications of: c-myc in promyelocytic leukemia cell line HL60, and in small-cell lung carcinoma cell lines, N-myc in primary neuroblastomas (stages Ill and IV), neuroblastoma cell lines, retinoblastoma cell line and primary tumors, and small-cell lung carcinoma lines and tumors, L-myc in small-cell lung carcinoma cell lines and tumors, c-myb in acute myeloid leukemia and in colon carcinoma cell lines, c-erbb in epidermoid carcinoma cell, and primary gliomas, c-K-ras-2 in primary carcinomas of lung, colon, bladder, and rectum, N-ras in mammary carcinoma cell line (Varmus H., Ann Rev Genetics 18: 553-612 (1984) [cited in Watson et al., Molecular Biology of the Gene (4th ed.; Benjamin/Cummings Publishing Co. 1987)].

Duplications of oncogenes are a common cause of many types of cancer, as is the case with P70-S6 Kinase 1 amplification and breast cancer. In such cases the genetic duplication occurs in a somatic cell and affects only the genome of the cancer cells themselves, not the entire organism, much less any subsequent offspring. Other examples of oncogenes that are amplified in human cancers include MYC, ERBB2 (EFGR), CCND1 (Cyclin D1), FGFR1 and FGFR2 in breast cancer, MYC and ERBB2 in cervical cancer, HRAS, KRAS, and MYB in colorectal cancer, MYC, CCND1 and MDM2 in esophageal cancer, CCNE, KRAS and MET in gastric cancer, ERBB1, and CDK4 in glioblastoma, CCND1, ERBB1, and MYC in head and neck cancer, CCND1 in hepatocellular cancer, MYCB in neuroblastoma, MYC, ERBB2 and AKT2 in ovarian cancer, MDM2 and CDK4 in sarcoma, and MYC in small cell lung cancer. In one embodiment, the present method can be used to determine the presence or absence of amplification of an oncogene associated with a cancer. In some embodiments, the amplified oncogene is associated with breast cancer, cervical cancer, colorectal cancer, esophageal cancer, gastric cancer, glioblastoma, head and neck cancer, hepatocellular cancer, neuroblastoma, ovarian cancer, sarcoma, and small cell lung cancer.

In one embodiment, the present method can be used to determine the presence or absence of a chromosomal deletion. In some embodiments, the chromosomal deletion is the loss of one or more entire chromosomes. In other embodiments, the chromosomal deletion is the loss of one or more segments of a chromosome. In yet other embodiments, the chromosomal deletion is the loss of two or more segments of two or more chromosomes. The chromosomal deletion can involve the loss of one or more tumor suppressor genes.

Chromosomal deletions involving tumor suppressor genes are believed to play an important role in the development and progression of solid tumors. The retinoblastoma tumor suppressor gene (Rb-1), located in chromosome 13q14, is the most extensively characterized tumor suppressor gene. The Rb-1 gene product, a 105 kDa nuclear phosphoprotein, apparently plays an important role in cell cycle regulation (Howe et al., Proc Natl Acad Sci (USA) 87:5883-5887 [1990]). Altered or lost expression of the Rb protein is caused by inactivation of both gene alleles either through a point mutation or a chromosomal deletion. Rb-i gene alterations have been found to be present not only in retinoblastomas but also in other malignancies such as osteosarcomas, small cell lung cancer (Rygaard et al., Cancer Res 50: 5312-5317 [1990)]) and breast cancer. Restriction fragment length polymorphism (RFLP) studies have indicated that such tumor types have frequently lost heterozygosity at 13q suggesting that one of the Rb-1 gene alleles has been lost due to a gross chromosomal deletion (Bowcock et al., Am J Hum Genet, 46: 12 [1990]). Chromosome 1 abnormalities including duplications, deletions and unbalanced translocations involving chromosome 6 and other partner chromosomes indicate that regions of chromosome 1, in particular 1q21-1q32 and 1 p11-13, might harbor oncogenes or tumor suppressor genes that are pathogenetically relevant to both chronic and advanced phases of myeloproliferative neoplasms (Caramazza et al., Eur J Hematol 84:191-200 [2010]). Myeloproliferative neoplasms are also associated with deletions of chromosome 5. Complete loss or interstitial deletions of chromosome 5 are the most common karyotypic abnormality in myelodysplastic syndromes (MDSs). Isolated del(5q)/5q-MDS patients have a more favorable prognosis than those with additional karyotypic defects, who tend to develop myeloproliferative neoplasms (MPNs) and acute myeloid leukemia. The frequency of unbalanced chromosome 5 deletions has led to the idea that 5q harbors one or more tumor-suppressor genes that have fundamental roles in the growth control of hematopoietic stem/progenitor cells (HSCs/HPCs). Cytogenetic mapping of commonly deleted regions (CDRs) centered on 5q31 and 5q32 identified candidate tumor-suppressor genes, including the ribosomal subunit RPS14, the transcription factor Egr1/Krox20 and the cytoskeletal remodeling protein, alpha-catenin (Eisenmann et al., Oncogene 28:3429-3441 [2009]). Cytogenetic and allelotyping studies of fresh tumors and tumor cell lines have shown that allelic loss from several distinct regions on chromosome 3p, including 3p25, 3p21-22, 3p21.3, 3p12-13 and 3p14, are the earliest and most frequent genomic abnormalities involved in a wide spectrum of major epithelial cancers of lung, breast, kidney, head and neck, ovary, cervix, colon, pancreas, esophagus, bladder and other organs. Several tumor suppressor genes have been mapped to the chromosome 3p region, and are thought that interstitial deletions or promoter hypermethylation precede the loss of the 3p or the entire chromosome 3 in the development of carcinomas (Angeloni D., Briefings Functional Genomics 6:19-39 [2007]).

Newborns and children with Down syndrome (DS) often present with congenital transient leukemia and have an increased risk of acute myeloid leukemia and acute lymphoblastic leukemia. Chromosome 21, harboring about 300 genes, may be involved in numerous structural aberrations, e.g., translocations, deletions, and amplifications, in leukemias, lymphomas, and solid tumors. Moreover, genes located on chromosome 21 have been identified that play an important role in tumorigenesis. Somatic numerical as well as structural chromosome 21 aberrations are associated with leukemias, and specific genes including RUNX1, TMPRSS2, and TFF, which are located in 21q, play a role in tumorigenesis (Fonatsch C Gene Chromosomes Cancer 49:497-508 [2010]).

In view of the foregoing, in various embodiments, the methods described herein can be used to determine the segment CNVs that are known to comprise one or more oncogenes or tumor suppressor genes, and/or that are known to be associated with a cancer or an increased risk of cancer. In certain embodiments, the CNVs can be determined in a test sample comprising a constitutional (germline) nucleic acid and the segment can be identified in those constitutional nucleic acids. In certain embodiments, segment CNVs are identified (if present) in a sample comprising a mixture of nucleic acids (e.g., nucleic acids derived from normal and nucleic acids derived from neoplastic cells). In certain embodiments, the sample is derived from a subject that is suspected or is known to have cancer e.g., carcinoma, sarcoma, lymphoma, leukemia, germ cell tumors, blastoma, and the like. In one embodiment, the sample is a plasma sample derived (processed) from peripheral blood that may comprise a mixture of cfDNA derived from normal and cancerous cells. In another embodiment, the biological sample that is used to determine whether a CNV is present is derived from a cells that, if a cancer is present, comprises a mixture of cancerous and non-cancerous cells from other biological tissues including, but not limited to biological fluids such as serum, sweat, tears, sputum, urine, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, and leukophoresis samples, or in tissue biopsies, swabs, or smears. In other embodiments, the biological sample is a stool (fecal) sample.

The CNVs used to determine presence of a cancer and/or increased risk for a cancer can comprise amplification or deletions.

In various embodiments, the CNVs identified as indicative of the presence of a cancer or an increased risk for a cancer include one or more of the amplifications shown in Table 3.

TABLE 3
Illustrative, but non-limiting chromosomal segments characterized
by amplification that are associated with cancers; cancers types
listed are those identified in Beroukhim et al. Nature 18: 463: 899-905

| Peak | Length | Cancer types identified in this |
|---|---|---|
| region | (Mb) | analysis but not prior publications |
| chr1: 119996566- | 0.228 | Breast, Lung SC, Melanoma |
| 120303234 | | |
| chr1: 148661965- | 0.35 | Breast, Dedifferentiated liposarcoma, |
| 149063439 | | Esophageal adenocarcinoma, |
| | | Hepatocellular, Lung SC, Melanoma, |
| | | Ovarian, Prostate, Renal |
| chr1: 1-5160566 | 4.416 | Esophageal adenocarcinoma, Ovarian |
| chr1: 158317017- | 1.627 | Dedifferentiated liposarcoma, |
| 159953843 | | Esophageal adenocarcinoma, Prostate, |
| | | Renal |
| chr1: 169549478- | 0.889 | Colorectal, Dedifferentiated |
| 170484405 | | liposarcoma, Prostate, Renal |
| chr1: 201678483- | 1.471 | Prostate |
| 203358272 | | |
| chr1: 241364021- | 5.678 | Lung NSC, Melanoma, Ovarian |
| 247249719 | | |
| chr1: 39907605- | 0.319 | Acute lymphoblastic leukemia, |
| 40263248 | | Breast, Lung NSC, Lung SC |
| chr1: 58658784- | 1.544 | Breast, Dedifferentiated liposarcoma, |
| 60221344 | | Lung SC |
| chr3: 170024984- | 3.496 | Breast, Esophageal adenocarcinoma, |
| 173604597 | | Glioma |
| chr3: 178149984- | 21.123 | Esophageal squamous, Lung NSC |
| 199501827 | | |
| chr3: 8620885- | 8.795 | Lung SC, Melanoma |
| 95164178 | | |
| chr4: 54471680- | 1.449 | Lung NSC |
| 55980061 | | |
| chr5: 1212750- | 0.115 | Dedifferentiated liposarcoma |
| 1378766 | | |
| chr5: 174477192- | 6.124 | Breast, Lung NSC |
| 180857866 | | |
| chr5: 45312870- | 4.206 | Lung SC |
| 49697231 | | |
| chr6: 1-23628840 | 23.516 | Esophageal adenocarcinoma |
| chr6: 135561194- | 0.092 | Breast, Esophageal adenocarcinoma |
| 135665525 | | |
| chr6: 43556800- | 0.72 | Esophageal adenocarcinoma, |
| 44361368 | | Hepatocellular, Ovarian |
| chr6: 63255006- | 1.988 | Esophageal adenocarcinoma, |
| 65243766 | | Lung NSC |
| chr7: 115981465- | 0.69 | Esophageal adenocarcinoma, Lung |
| 116676953 | | NSC, Melanoma, Ovarian |
| chr7: 54899301- | 0.363 | Esophageal adenocarcinoma, |
| 55275419 | | Esophageal squamous |
| chr7: 89924533- | 9.068 | Breast, Esophageal adenocarcinoma, |
| 98997268 | | Esophageal squamous, Ovarian |
| chr8: 101163387- | 2.516 | Lung NSC, Melanoma, Ovarian |
| 103693879 | | |
| chr8: 116186189- | 4.4 | Breast, Hepatocellular, Lung NSC, |
| 120600761 | | Ovarian |
| chr8: 128774432- | 0.009 | Esophageal adenocarcinoma, |
| 128849112 | | Esophageal squamous, Hepatocellular, |
| | | Lung SC, Medulloblastoma, |
| | | Myeloproliferative disorder, Ovarian |
| chr8: 140458177- | 5.784 | Lung NSC, Medulloblastoma, |
| 146274826 | | Melanoma, Ovarian |
| chr8: 38252951- | 0.167 | Colorecal, Esophageal adenocarcinoma, |
| 38460772 | | Esophageal squamous |
| chr8: 42006632- | 0.257 | Esophageal adenocarcinoma, Lung NSC, |
| 42404492 | | Lung SC, Ovarian, Prostate |
| chr8: 81242335- | 0.717 | Breast, Melanoma |
| 81979194 | | |
| chr9: 137859478- | 2.29 | Colorectal, Dedifferentiated liposarcoma |
| 140273252 | | |
| chr10: 74560456- | 7.455 | Breast, Ovarian, Prostate |
| 82020637 | | |
| chr11: 101433436- | 0.683 | Lung NSC, Lung SC |
| 102134907 | | |
| chr11: 32027116- | 5.744 | Breast, Dedifferentiated liposarcoma, |
| 37799354 | | Lung NSC, Lung SC |
| chr11: 69098089- | 0.161 | Dedifferentiated liposarcoma, |
| 69278404 | | Esophageal adenocarcinoma, |
| | | Hepatocellular, Lung SC, Ovarian |
| chr11: 76699529- | 1.286 | Dedifferentiated liposarcoma, |
| 78005085 | | Esophageal adenosarcoma, Lung SC, |
| | | Ovarian |
| chr12: 1-1311104 | 1.271 | Lung NSC |
| chr12: 25189655- | 0.112 | Acute lymphoblastic leukemia, |
| 25352305 | | Esophegeal adenocarcinoma, Esophageal |
| | | squamous, Ovarian |
| chr12: 30999223- | 1.577 | Acute lymphoblastic leukemia, |
| 32594050 | | Colorectal, Esophageal adenocarcinoma, |
| | | Esophageal squamous, Lung NSC, |
| | | Lung SC |
| chr12: 38788913- | 3.779 | Breast, Colorectal, Dedifferentiated |
| 42596599 | | liposarcoma, Esophageal squamous, |
| | | Lung NSC, Lung SC |
| chr12: 56419524- | 0.021 | Dedifferentiated liposarcoma, |
| 56488685 | | Melanoma, Renal |
| chr12: 64461446- | 0.041 | Dedifferentiated liposarcoma, Renal |
| 64607139 | | |
| chr12: 66458200- | 0.058 | Dedifferentiated liposarcoma, |
| 66543552 | | Esophageal squamous, Renal |
| chr12: 67440273- | 0.067 | Breast, Dedifferentiated liposarcoma, |
| 67566002 | | Esophageal squamous, Melanoma, Renal |
| chr12: 68249634- | 0.06 | Breast, Dedifferentiated liposarcoma, |
| 68327233 | | Esophageal squamous, Renal |
| chr12: 70849987- | 0.036 | Dedifferentiated liposarcoma, Renal |
| 70966467 | | |
| chr12: 72596017- | 0.23 | Renal |
| 73080626 | | |
| chr12: 76852527- | 0.158 | Dedifferentiated liposarcoma |
| 77054746 | | |
| chr12: 85072329- | 0.272 | Dedifferentiated liposarcoma |
| 85674601 | | |
| chr12: 95089777- | 0.161 | Dedifferentiated liposarcoma |
| 95350380 | | |
| chr13: 108477140- | 1.6 | Breast, Esophageal adenocarcinoma, |
| 110084607 | | Lung NSC, Lung SC |
| chr13: 1-40829685 | 22.732 | Acute lymphoblastic leukemia, |
| | | Esophageal adenocarcinoma |
| chr13: 89500014- | 3.597 | Breast, Esophageal adenocarcinoma, |
| 93206506 | | Medulloblastoma |
| chr14: 106074644- | 0.203 | Esophageal squamous |
| 106368585 | | |
| chr14: 1-23145193 | 3.635 | Acute lymphoblastic leukemia, |
| | | Esophageal squamous, Hepatocellular, |
| | | Lung SC |
| chr14: 35708407- | 0.383 | Breast, Esophageal adenocarcinoma, |
| 36097605 | | Esophageal squamous, Hepatocellular, |
| | | Prostate |
| chr15: 96891354- | 0.778 | Breast, Colorectal, Esophageal |
| 97698742 | | adenocarcinoma, Lung NSC, |
| | | Medulloblastoma, Melanoma |
| chr17: 18837023- | 0.815 | Breast, Hepatocellular |
| 19933105 | | |
| chr17: 22479313- | 0.382 | Breast, Lung NSC |
| 22877776 | | |
| chr17: 24112056- | 0.114 | Breast, Lung NSC |
| 24310787 | | |
| chr17: 35067383- | 0.149 | Colorectal, Esophageal adenocarcinoma, |
| 35272328 | | Esophageal squamous |
| chr17: 44673157- | 0.351 | Melanoma |
| 45060263 | | |
| chr17: 55144989- | 0.31 | Lung NSC, Medulloblastoma, |
| 55540417 | | Melanoma, Ovarian |
| chr17: 62318152- | 1.519 | Breast, Lung NSC, Melanoma, Ovarian |
| 63890591 | | |
| chr17: 70767943- | 0.537 | Breast, Lung NSC, Melanoma, Ovarian |
| 71305641 | | |
| chr18: 17749667- | 5.029 | Colorectal, Esophageal adenocarcinoma, |
| 22797232 | | Ovarian |
| chr19: 34975531- | 0.096 | Breast, Esophageal adenocarcinoma, |
| 35098303 | | Esophageal squamous |
| chr19: 43177306- | 2.17 | Lung NSC, Ovarian |
| 45393020 | | |
| chr19: 59066340- | 0.321 | Breast, Lung NSC, Ovarian |
| 59471027 | | |
| chr2: 15977811- | 0.056 | Lung SC |
| 16073001 | | |
| chr20: 29526118- | 0.246 | Ovarian |
| 29834552 | | |
| chr20: 51603033- | 0.371 | Hepatocellular, Lung NSC, Ovarian |
| 51989829 | | |
| chr20: 61329497- | 0.935 | Hepatocellular, Lung NSC |
| 62435964 | | |
| chr22: 19172385- | 0.487 | Colorectal, Melanoma, Ovarian |
| 19746441 | | |
| chrX: 152729030- | 1.748 | Breast, Lung NSC, Renal |
| 154913754 | | |
| chrX: 66436234- | 0.267 | Ovarian, Prostate |
| 67090514 | | |

In certain embodiments, in combination with the amplifications described above (herein), or separately, the CNVs identified as indicative of the presence of a cancer or an increased risk for a cancer include one or more of the deletions shown in Table 4.

TABLE 4
Illustrative, but non-limiting chromosomal segments characterized by deletions
that are associated with cancers; cancer types listed are those identified in
Beroukhim et al. Nature 18: 463: 899-905

| | Length | Cancer types identified in this analysis but |
|---|---|---|
| Peak region | (Mb) | not prior publications |
| chr1: 110339388- | 1p13.2 | Acute lymphoblastic leukemia, Esophageal |
| 119426489 | | adenocarcinoma, Lung NSC, Lung SC, |
| | | Melanoma, Ovarian, Prostate |
| chr1: 223876038- | 1q43 | Acute lymphoblastic leukemia, Breast, Lung |
| 247249719 | | SC, Melanoma, Prostate |
| chr1: 26377344- | 1p36.11 | Breast, Esophageal adenocarcinoma, |
| 27532551 | | Esophageal squamous, Lung NSC, Lung SC, |
| | | Medulloblastoma, Myeloproliferative disorder, |
| | | Ovarian, Prostate |
| chr1: 3756302- | 1p36.31 | Acute lymphoblastic leukemia, Breast, |
| 6867390 | | Esophageal squamous, Hepatocellular, Lung |
| | | NSC, Lung SC, Medulloblastoma, |
| | | Myeloproliferative disorder, Ovarian, Prostate, |
| | | Renal |
| chr1: 71284749- | 1p31.1 | Breast, Esophageal adenocarcinoma, Glioma, |
| 74440273 | | Hepatocellular, Lung NSC, Lung SC, |
| | | Melanoma, Ovarian, Renal |
| chr2: 1-15244284 | 2p25.3 | Lung NSC, Ovarian |
| chr2: 138479322- | 2q22.1 | Breast, Colorectal, Esophageal |
| 143365272 | | adenocarcinoma, Esophageal squamous, |
| | | Hepatocellular, Lung NSC, Ovarian, Prostate, |
| | | Renal |
| chr2: 204533830- | 2q33.2 | Esophageal adenocarcinoma, Hepatocellular, |
| 206266883 | | Lung NSC, Medulloblastoma, Renal |
| chr2: 241477619- | 2q37.3 | Breast, Dedifferentiated liposarcoma, |
| 242951149 | | Esophageal adenocarcinoma, Esophageal |
| | | squamous, Hepatocellular, Lung NSC, Lung |
| | | SC, Medulloblastoma, Melanoma, Ovarian, |
| | | Renal |
| chr3: 116900556- | 3q13.31 | Dedifferentiated liposarcoma, Esophageal |
| 120107320 | | adenocarcinoma, Hepatocellular, Lung NSC, |
| | | Melanoma, Myeloproliferative disorder, |
| | | Prostate |
| chr3: 1-2121282 | 3p26.3 | Colorectal, Dedifferentiated liposarcoma, |
| | | Esophageal adenocarcinoma, Lung NSC, |
| | | Melanoma, Myeloproliferative disorder |
| chr3: 175446835- | 3q26.31 | Acute lymphoblastic leukemia, Dedifferentiated |
| 178263192 | | liposarcoma, Esophageal adenocarcinoma, |
| | | Lung NSC, Melanoma, Myeloproliferative |
| | | disorder, Prostate |
| chr3: 58626894- | 3p14.2 | Breast, Colorectal, Dedifferentiated |
| 61524607 | | liposarcoma, Esophageal adenocarcinoma, |
| | | Esophageal squamous, Hepatocellular, Lung |
| | | NSC, Lung SC, Medulloblastoma, Melanoma, |
| | | Myeloproliferative disorder, Ovarian, Prostate, |
| | | Renal |
| chr4: 1-435793 | 4p16.3 | Myeloproliferative disorder |
| chr4: 186684565- | 4q35.2 | Breast, Esophageal adenocarcinoma, |
| 191273063 | | Esophageal squamous, Lung NSC, |
| | | Medulloblastoma, Melanoma, Prostate, Renal |
| chr4: 91089383- | 4q22.1 | Acute lymphoblastic leukemia, Esophageal |
| 93486891 | | adenocarcinoma, Hepatocellular, Lung NSC, |
| | | Renal |
| chr5: 177541057- | 5q35.3 | Breast, Lung NSC, Myeloproliferative disorder, |
| 180857866 | | Ovarian |
| chr5: 57754754- | 5q11.2 | Breast, Colorectal, Dedifferentiated |
| 59053198 | | liposarcoma, Esophageal adenocarcinoma, |
| | | Esophageal squamous, Lung SC, Melanoma, |
| | | Myeloproliferative disorder, Ovarian, Prostate |
| chr5: 85837489- | 5q21.1 | Colorectal, Dedifferentiated liposarcoma, Lung |
| 133480433 | | NSC, Lung SC, Myeloproliferative disorder, |
| | | Ovarian |
| chr6: 101000242- | 6q22.1 | Colorectal, Lung NSC, Lung SC |
| 121511318 | | |
| chr6: 1543157- | 6p25.3 | Colorectal, Dedifferentiated liposarcoma, |
| 2570302 | | Esophageal adenocarcinoma, Lung NSC, Lung |
| | | SC, Ovarian, Prostate |
| chr6: 161612277- | 6q26 | Colorectal, Esophageal adenocarcinoma, |
| 163134099 | | Esophageal squamous, Lung NSC, Lung SC, |
| | | Ovarian, Prostate |
| chr6: 76630464- | 6q16.1 | Colorectal, Hepatocellular, Lung NSC |
| 105342994 | | |
| chr7: 141592807- | 7q34 | Breast, Colorectal, Esophageal |
| 142264966 | | adenocarcinoma, Esophageal squamous, |
| | | Hepatocellular, Lung NSC, Ovarian, Prostate, |
| | | Renal |
| chr7: 144118814- | 7q35 | Breast, Esophageal adenocarcinoma, |
| 148066271 | | Esophageal squamous, Lung NSC, Melanoma, |
| | | Myeloproliferative disorder, Ovarian |
| chr7: 156893473- | 7q36.3 | Breast, Esophageal adenocarcinoma, |
| 158821424 | | Esophageal squamous, Lung NSC, Melanoma, |
| | | Myeloproliferative disorder, Ovarian, Prostate |
| chr7: 3046420- | 7p22.2 | Melanoma, Myeloproliferative disorder, Ovarian |
| 4279470 | | |
| chr7: 65877239- | 7q21.11 | Breast, Medulloblastoma, Melanoma, |
| 79629882 | | Myeloproliferative disorder, Ovarian |
| chr8: 1-392555 | 8p23.3 | Acute lymphoblastic leukemia, Breast, |
| | | Myeloproliferative disorder |
| chr8: 2053441- | 8p23.2 | Acute lymphoblastic leukemia, Dedifferentiated |
| 6259545 | | liposarcoma, Esophageal adenocarcinoma, |
| | | Esophageal squamous, Hepatocellular, Lung |
| | | NSC, Myeloproliferative disorder |
| chr8: 22125332- | 8p21.2 | Acute lymphoblastic leukemia, Dedifferentiated |
| 30139123 | | liposarcoma, Hepatocellular, Myeloproliferative |
| | | disorder, Ovarian, Renal |
| chr8: 39008109- | 8p11.22 | Acute lymphoblastic leukemia, Breast, |
| 41238710 | | Dedifferentiated liposarcoma, Esophageal |
| | | squamous, Hepatocellular, Lung NSC, |
| | | Myeloproliferative disorder, Renal |
| chr8: 42971602- | 8q11.22 | Breast, Dedifferentiated liposarcoma, |
| 72924037 | | Esophageal squamous, Hepatocellular, Lung |
| | | NSC, Myeloproliferative disorder, Renal |
| chr9: 1-708871 | 9p24.3 | Acute lymphoblastic leukemia, Breast, Lung |
| | | NSC, Myeloproliferative disorder, Ovarian, |
| | | Prostate |
| chr9: 21489625- | 9p21.3 | Colorectal, Esophageal adenocarcinoma, |
| 22474701 | | Esophageal squamous, Myeloproliferative |
| | | disorder, Ovarian |
| chr9: 36365710- | 9p13.2 | Myeloproliferative disorder |
| 37139941 | | |
| chr9: 7161607- | 9p24.1 | Acute lymphoblastic leukemia, Breast, |
| 12713130 | | Colorectal, Esophageal adenocarcinoma, |
| | | Hepatocellular, Lung SC, Medulloblastoma, |
| | | Melanoma, Myeloproliferative disorder, |
| | | Ovarian, Prostate, Renal |
| chr10: 1-1042949 | 10p15.3 | Colorectal, Lung NSC, Lung SC, Ovarian, |
| | | Prostate, Renal |
| chr10: 129812260- | 10q26.3 | Breast, Colorectal, Glioma, Lung NSC, Lung |
| 135374737 | | SC, Melanoma, Ovarian, Renal |
| chr10: 52313829- | 10q11.23 | Colorectal, Lung NSC, Lung SC, Ovarian, |
| 53768264 | | Renal |
| chr10: 89467202- | 10q23.31 | Breast, Lung SC, Ovarian, Renal |
| 90419015 | | |
| chr11: 107086196- | 11q23.1 | Esophageal adenocarcinoma, |
| 116175885 | | Medulloblastoma, Renal |
| chr11: 1-1391954 | 11p15.5 | Breast, Dedifferentiated liposarcoma, |
| | | Esophageal adenocarcinoma, Lung NSC, |
| | | Medulloblastoma, Ovarian |
| chr11: 130280899- | 11q25 | Esophageal adenocarcinoma, Esophageal |
| 134452384 | | squamous, Hepatocellular, Lung NSC, |
| | | Medulloblastoma, Renal |
| chr11: 82612034- | 11q14.1 | Melanoma, Renal |
| 85091467 | | |
| chr12: 11410696- | 12p13.2 | Breast, Hepatocellular, Myeloproliferative |
| 12118386 | | disorder, Prostate |
| chr12: 131913408- | 12q24.33 | Dedifferentiated liposarcoma, Lung NSC, |
| 132349534 | | Myeloproliferative disorder |
| chr12: 97551177- | 12q23.1 | Breast, Colorectal, Esophageal squamous, |
| 99047626 | | Lung NSC, Myeloproliferative disorder |
| chr13: 111767404- | 13q34 | Breast, Hepatocellular, Lung NSC |
| 114142980 | | |
| chr13: 1-23902184 | 13q12.11 | Breast, Lung SC, Ovarian |
| chr13: 46362859- | 13q14.2 | Hepatocellular, Lung SC, Myeloproliferative |
| 48209064 | | disorder, Prostate |
| chr13: 92308911- | 13q31.3 | Breast, Hepatocellular, Lung NSC, Renal |
| 94031607 | | |
| chr14: 1-29140968 | 14q11.2 | Acute lymphoblastic leukemia, Esophageal |
| | | adenocarcinoma, Myeloproliferative disorder |
| chr14: 65275722- | 14q23.3 | Dedifferentiated liposarcoma, |
| 67085224 | | Myeloproliferative disorder |
| chr14: 80741860- | 14q32.12 | Acute lymphoblastic leukemia, Dedifferentiated |
| 106368585 | | liposarcoma, Melanoma, Myeloproliferative |
| | | disorder |
| chr15: 1-24740084 | 15q11.2 | Acute lymphoblastic leukemia, Breast, |
| | | Esophageal adenocarcinoma, Lung NSC, |
| | | Myeloproliferative disorder, Ovarian |
| chr15: 35140533- | 15q15.1 | Esophageal adenocarcinoma, Lung NSC, |
| 43473382 | | Myeloproliferative disorder |
| chr16: 1-359092 | 16p13.3 | Esophageal adenocarcinoma, Hepatocellular, |
| | | Lung NSC, Renal |
| chr16: 31854743- | 16q11.2 | Breast, Hepatocellular, Lung NSC, Melanoma, |
| 53525739 | | Renal |
| chr16: 5062786- | 16p13.3 | Hepatocellular, Lung NSC, Medulloblastoma, |
| 7709383 | | Melanoma, Myeloproliferative disorder, |
| | | Ovarian, Renal |
| chr16: 76685816- | 16q23.1 | Breast, Colorectal, Esophageal |
| 78205652 | | adenocarcinoma, Hepatocellular, Lung NSC, |
| | | Lung SC, Medulloblastoma, Renal |
| chr16: 80759878- | 16q23.3 | Colorectal, Hepatocellular, Renal |
| 82408573 | | |
| chr16: 88436931- | 16q24.3 | Colorectal, Hepatocellular, Lung NSC, |
| 88827254 | | Prostate, Renal |
| chr17: 10675416- | 17p12 | Lung NSC, Lung SC, Myeloproliferative |
| 12635879 | | disorder |
| chr17: 26185485- | 17q11.2 | Breast, Colorectal, Dedifferentiated |
| 27216066 | | liposarcoma, Lung NSC, Lung SC, Melanoma, |
| | | Myeloproliferative disorder, Ovarian |
| chr17: 37319013- | 17q21.2 | Breast, Colorectal, Dedifferentiated |
| 37988602 | | liposarcoma, Lung SC, Melanoma, |
| | | Myeloproliferative disorder, Ovarian |
| chr17: 7471230- | 17p13.1 | Lung SC, Myeloproliferative disorder |
| 7717938 | | |
| chr17: 78087533- | 17q25.3 | Colorectal, Myeloproliferative disorder |
| 78774742 | | |
| chr18: 1-587750 | 18p11.32 | Myeloproliferative disorder |
| chr18: 46172638- | 18q21.2 | Esophageal adenocarcinoma, Lung NSC |
| 49935241 | | |
| chr18: 75796373- | 18q23 | Colorectal, Esophageal adenocarcinoma, |
| 76117153 | | Esophageal squamous, Ovarian, Prostate |
| chr19: 1-526082 | 19p13.3 | Hepatocellular, Lung NSC, Renal |
| chr19: 21788507- | 19p12 | Hepatocellular, Lung NSC, Renal |
| 34401877 | | |
| chr19: 52031294- | 19q13.32 | Breast, Hepatocellular, Lung NSC, |
| 53331283 | | Medulloblastoma, Ovarian, Renal |
| chr19: 63402921- | 19q13.43 | Breast, Colorectal, Dedifferentiated |
| 63811651 | | liposarcoma, Hepatocellular, Lung NSC, |
| | | Medulloblastoma, Ovarian, Renal |
| chr20: 1-325978 | 20p13 | Breast, Dedifferentiated liposarcoma, Lung |
| | | NSC |
| chr20: 14210829- | 20p12.1 | Esophageal adenocarcinoma, Lung NSC, |
| 15988895 | | Medulloblastoma, Melanoma, |
| | | Myeloproliferative disorder, Prostate, Renal |
| chr21: 38584860- | 21q22.2 | Breast |
| 42033506 | | |
| chr22: 20517661- | 22q11.22 | Acute lymphoblastic leukemia, Esophageal |
| 21169423 | | adenocarcinoma |
| chr22: 45488286- | 22q13.33 | Breast, Hepatocellular, Lung NSC, Lung SC |
| 49691432 | | |
| chrX: 1-3243111 | Xp22.33 | Esophageal adenocarcinoma, Lung NSC, Lung |
| | | SC |
| chrX: 31041721- | Xp21.2 | Acute lymphoblastic leukemia, Esophageal |
| 34564697 | | adenocarcinoma, Glioma |

The aneuploidies identified as characteristic of various cancers (e.g., the aneuploidies identified in Tables 4 and 5) may contain genes known to be implicated in cancer etiologies (e.g., tumor suppressors, oncogenes, and the like). These aneuploidies can also be probed to identify relevant but previously unknown genes.

For example, Beroukhim et al. supra, assessed potential cancer-causing genes in the copy number alterations using GRAIL (Gene Relationships Among Implicated Loci20), an algorithm that searches for functional relationships among genomic regions. GRAIL scores each gene in a collection of genomic regions for its ‘relatedness’ to genes in other regions based on textual similarity between published abstracts for all papers citing the genes, on the notion that some target genes will function in common pathways. These methods permit identification/characterization of genes previously not associated with the particular cancers at issue. Table 5 illustrates target genes known to be within the identified amplified segment and predicted genes, and Table 6 illustrates target genes known to be within the identified deleted segment and predicted genes.

TABLE 5
Illustrative, but non-limiting chromosomal segments and genes known or
predicted to be present in regions characterized by amplification
in various cancers (see, e.g., Beroukhim et al. supra.)

| Chromosome | | | Known | GRAIL top |
|---|---|---|---|---|
| and band | Peak region | # genes | target | target |
| 8q24.21 | chr8: 128774432- | 1 | MYC | MYC |
| | 128849112 | | | |
| 11q13.2 | chr11: 69098089- | 3 | CCND1 | ORAOV1 |
| | 69278404 | | | |
| 17q12 | chr17: 35067383- | 6 | ERBB2 | ERBB2, |
| | 35272328 | | | C17orf37 |
| 12q14.1 | chr12: 56419524- | 7 | CDK4 | TSPAN31 |
| | 56488685 | | | |
| 14q13.3 | chr14: 35708407- | 3 | NKX2-1 | NKX2-1 |
| | 36097605 | | | |
| 12q15 | chr12: 67440273- | 1 | MDM2 | MDM2 |
| | 67566002 | | | |
| 7p11.2 | chr7: 54899301- | 1 | EGFR | EGFR |
| | 55275419 | | | |
| 1q21.2 | chr1: 148661965- | 9 | MCL1‡ | MCL1 |
| | 149063439 | | | |
| 8p12 | chr8: 38252951- | 3 | FGFR1 | FGFR1 |
| | 38460772 | | | |
| 12p12.1 | chr12: 25189655- | 2 | KRAS | KRAS |
| | 25352305 | | | |
| 19q12 | chr19: 34975531- | 1 | CCNE1 | CCNE1 |
| | 35098303 | | | |
| 22q11.21 | chr22: 19172385- | 11 | CRKL | CRKL |
| | 19746441 | | | |
| 12q15 | chr12: 68249634- | 2 | | LRRC10 |
| | 68327233 | | | |
| 12q14.3 | chr12: 64461446- | 1 | HMGA2 | HMGA2 |
| | 64607139 | | | |
| Xq28 | chrX: 152729030- | 53 | | SPRY3 |
| | 154913754 | | | |
| 5p15.33 | chr5: 1212750- | 3 | TERT | TERT |
| | 1378766 | | | |
| 3q26.2 | chr3: 170024984- | 22 | PRKCI | PRKCI |
| | 173604597 | | | |
| 15q26.3 | chr15: 96891354- | 4 | IGF1R | IGF1R |
| | 97698742 | | | |
| 20q13.2 | chr20: 51603033- | 1 | | ZNF217 |
| | 51989829 | | | |
| 8p11.21 | chr8: 42006632- | 6 | | PLAT |
| | 42404492 | | | |
| 1p34.2 | chr1: 39907605- | 7 | MYCL1 | MYCL1 |
| | 40263248 | | | |
| 17q21.33 | chr17: 44673157- | 4 | | NGFR, |
| | 45060263 | | | PHB |
| 2p24.3 | chr2: 15977811- | 1 | MYCN | MYCN |
| | 16073001 | | | |
| 7q21.3 | chr7: 89924533- | 62 | CDK6 | CDK6 |
| | 98997268 | | | |
| 13q34 | chr13: 108477140- | 4 | | IRS2 |
| | 110084607 | | | |
| 11q14.1 | chr11: 76699529- | 14 | | GAB2 |
| | 78005085 | | | |
| 20q13.33 | chr20: 61329497- | 38 | | BIRC7 |
| | 62435964 | | | |
| 17q23.1 | chr17: 55144989- | 5 | | RPS6KB1 |
| | 55540417 | | | |
| 1p12 | chr1: 119996566- | 5 | | REG4 |
| | 120303234 | | | |
| 8q21.13 | chr8: 81242335- | 3 | | ZNF704, |
| | 81979194 | | | ZBTB10 |
| 6p21.1 | chr6: 43556800- | 18 | | VEGFA |
| | 44361368 | | | |
| 5p11 | chr5: 45312870- | 0 | | |
| | 49697231 | | | |
| 20q11.21 | chr20: 29526118- | 5 | BCL2L1‡ | BCL2L1, |
| | 29834552 | | | ID1 |
| 6q23.3 | chr6: 135561194- | 1 | MYB | hsa-mir- |
| | 135665525 | | | 548a-2 |
| 1q44 | chr1: 241364021- | 71 | | AKT3 |
| | 247249719 | | | |
| 5q35.3 | chr5: 174477192- | 92 | | FLT4 |
| | 180857866 | | | |
| 7q31.2 | chr7: 115981465- | 3 | MET | MET |
| | 116676953 | | | |
| 18q11.2 | chr18: 17749667- | 21 | | CABLES1 |
| | 22797232 | | | |
| 17q25.1 | chr17: 70767943- | 13 | | GRB2, |
| | 71305641 | | | ITGB4 |
| 1p32.1 | chr1: 58658784- | 7 | JUN | JUN |
| | 60221344 | | | |
| 17q11.2 | chr17: 24112056- | 5 | | DHRS13, |
| | 24310787 | | | FLOT2, |
| | | | | ERAL1, |
| | | | | PHF12 |
| 17p11.2 | chr17: 18837023- | 12 | | MAPK7 |
| | 19933105 | | | |
| 8q24.11 | chr8: 116186189- | 13 | | NOV |
| | 120600761 | | | |
| 12q15 | chr12: 66458200- | 0 | | |
| | 66543552 | | | |
| 19q13.2 | chr19: 43177306- | 60 | | LGALS7, |
| | 45393020 | | | DYRK1B |
| 11q22.2 | chr11: 101433436- | 8 | BIRC2, YAP1 | BIRC2 |
| | 102134907 | | | |
| 4q12 | chr4: 54471680- | 7 | PDGFRA, | KDR, KIT |
| | 55980061 | | KIT | |
| 12p11.21 | chr12: 30999223- | 9 | | DDX11, |
| | 32594050 | | | FAM60A |
| 3q28 | chr3: 178149984- | 143 | PIK3CA | PIK3CA |
| | 199501827 | | | |
| 1p36.33 | chr1: 1-5160566 | 77 | | TP73 |
| 17q24.2 | chr17: 62318152- | 12 | | BPTF |
| | 63890591 | | | |
| 1q23.3 | chr1: 158317017- | 52 | | PEA15 |
| | 159953843 | | | |
| 1q24.3 | chr1: 169549478- | 6 | | BAT2D1, |
| | 170484405 | | | MYOC |
| 8q22.3 | chr8: 101163387- | 14 | | RRM2B |
| | 103693879 | | | |
| 13q31.3 | chr13: 89500014- | 3 | | GPC5 |
| | 93206506 | | | |
| 12q21.1 | chr12: 70849987- | 0 | | |
| | 70966467 | | | |
| 12p13.33 | chr12: 1-1311104 | 10 | | WNK1 |
| 12q21.2 | chr12: 76852527- | 0 | | |
| | 77064746 | | | |
| 1q32.1 | chr1: 201678483- | 21 | MDM4 | MDM4 |
| | 203358272 | | | |
| 19q13.42 | chr19: 59066340- | 19 | | PRKCG, |
| | 59471027 | | | TSEN34 |
| 12q12 | chr12: 38788913- | 12 | | ADAMTS20 |
| | 42596599 | | | |
| 12q23.1 | chr12: 95089777- | 2 | | ELK3 |
| | 95350380 | | | |
| 12q21.32 | chr12: 85072329- | 0 | | |
| | 85674601 | | | |
| 10q22.3 | chr10: 74560456- | 46 | | SFTPA1B |
| | 82020637 | | | |
| 3p11.1 | chr3: 86250885- | 8 | | POU1F1 |
| | 95164178 | | | |
| 17q11.1 | chr17: 22479313- | 1 | | WSB1 |
| | 22877776 | | | |
| 8q24.3 | chr8: 140458177- | 97 | | PTP4A3, |
| | 146274826 | | | MAFA, |
| | | | | PARP10 |
| Xq12 | chrX: 66436234- | 1 | AR | AR |
| | 67090514 | | | |
| 6q12 | chr6: 63255006- | 3 | | PTP4A1 |
| | 65243766 | | | |
| 14q11.2 | chr14: 1-23145193 | 95 | | BCL2L2 |
| 9q34.3 | chr9: 137859478- | 76 | | NRARP, |
| | 140273252 | | | MRPL41, |
| | | | | TRAF2, |
| | | | | LHX3 |
| 6p24.1 | chr6: 1-23628840 | 95 | | E2F3 |
| 13q12.2 | chr13: 1-40829685 | 110 | | FOXO1 |
| 12q21.1 | chr12: 72596017- | 0 | | |
| | 73080626 | | | |
| 14q32.33 | chr14: 106074644- | 0 | | |
| | 106368585 | | | |
| 11p13 | chr11: 32027116- | 35 | | WT1 |
| | 37799354 | | | |

TABLE 6
Illustrative, but non-limiting chromosomal segments and genes known or
predicted to be present in regions characterized by amplification
in various cancers (see, e.g., Beroukhim et al. supra.)

| Chromosome | | # | Known | GRAIL top |
|---|---|---|---|---|
| and band | Peak region | genes | target | target |
| 9p21.3 | chr9: 21489625- | 5 | CDKN2A/B | CDKN2A |
| | 22474701 | | | |
| 3p14.2 | chr3: 58626894- | 2 | FHIT§ | FHIT |
| | 61524607 | | | |
| 16q23.1 | chr16: 76685816- | 2 | WWOX§ | WWOX |
| | 78205652 | | | |
| 9p24.1 | chr9: 7161607- | 3 | PTPRD§ | PTPRD |
| | 12713130 | | | |
| 20p12.1 | chr20: 14210829- | 2 | MACROD2§ | FLRT3 |
| | 15988895 | | | |
| 6q26 | chr6: 161612277- | 1 | PARK2§ | PARK2 |
| | 163134099 | | | |
| 13q14.2 | chr13: 46362859- | 8 | RB1 | RB1 |
| | 48209064 | | | |
| 2q22.1 | chr2: 138479322- | 3 | LRP1B§ | LRP1B |
| | 143365272 | | | |
| 4q35.2 | chr4: 186684565- | 15 | | FRG2, |
| | 191273063 | | | TUBB4Q |
| 5q11.2 | chr5: 57754754- | 5 | PDE4D§ | PLK2, |
| | 59053198 | | | PDE4D |
| 16p13.3 | chr16: 5062786- | 2 | A2BP1§ | A2BP1 |
| | 7709383 | | | |
| 7q34 | chr7: 141592807- | 3 | TRB@\hat{ } | PRSS1 |
| | 142264966 | | | |
| 2q37.3 | chr2: 241477619- | 19 | | TMEM16G, |
| | 242951149 | | | ING5 |
| 19p13.3 | chr19: 1-526082 | 10 | | GZMM, |
| | | | | THEG, |
| | | | | PPAP2C, |
| | | | | C19orf20 |
| 10q23.31 | chr10: 89467202- | 4 | PTEN | PTEN |
| | 90419015 | | | |
| 8p23.2 | chr8: 2053441- | 1 | CSMD1§ | CSMD1 |
| | 6259545 | | | |
| 1p36.31 | chr1: 3756302- | 23 | | DFFB, |
| | 6867390 | | | ZBTB48, |
| | | | | AJAP1 |
| 4q22.1 | chr4: 91089383- | 2 | | MGC48628 |
| | 93486891 | | | |
| 18q23 | chr18: 75796373- | 4 | | PARD6G |
| | 76117153 | | | |
| 6p25.3 | chr6: 1543157- | 2 | | FOXC1 |
| | 2570302 | | | |
| 19q13.43 | chr19: 63402921- | 17 | | ZNF324 |
| | 63811651 | | | |
| Xp21.2 | chrX: 31041721- | 2 | DMD§ | DMD |
| | 34564697 | | | |
| 11q25 | chr11: 130280899 | 12 | OPCML§, | HNT |
| | -134452384 | | HNT§ | |
| 13q12.11 | chr13: 1- | 29 | | LATS2 |
| | 23902184 | | | |
| 22q13.33 | chr22: 45488286- | 38 | | TUBGCP6 |
| | 49691432 | | | |
| 15q11.2 | chr15: 1- | 20 | | A26B1 |
| | 24740084 | | | |
| 22q11.22 | chr22: 20517661- | 3 | | VPREB1 |
| | 21169423 | | | |
| 10q26.3 | chr10: 129812260 | 35 | | MGMT, |
| | -135374737 | | | SYCE1 |
| 12p13.2 | chr12: 11410696- | 2 | ETV6$ | ETV6 |
| | 12118386 | | | |
| 8p23.3 | chr8: 1-392555 | 2 | | ZNF596 |
| 1p36.11 | chr1: 26377344- | 24 | | SFN |
| | 27532551 | | | |
| 11p15.5 | chr11: 1-1391954 | 49 | | RASSF7 |
| 17q11.2 | chr17: 26185485- | 10 | NF1 | NF1 |
| | 27216066 | | | |
| 11q23.1 | chr11: 107086196- | 61 | ATM | CADM1 |
| | 116175885 | | | |
| 9p24.3 | chr9: 1-708871 | 5 | | FOXD4 |
| 10q11.23 | chr10: 52313829- | 4 | PRKG1§ | DKK1, |
| | 53768264 | | | PRKG1 |
| 15q15.1 | chr15: 35140533- | 109 | | TUBGCP4 |
| | 43473382 | | | |
| 1p13.2 | chr1: 110339388- | 81 | | MAGI3 |
| | 119426489 | | | |
| Xp22.33 | chrX: 1-3243111 | 21 | | SHOX |
| 3p26.3 | chr3: 1-2121282 | 2 | | CHL1 |
| 9p13.2 | chr9: 36365710- | 2 | PAX5 | MELK |
| | 37139941 | | | |
| 17p13.1 | chr17: 7471230- | 10 | TP53 | ATP1B2 |
| | 7717938 | | | |
| 12q24.33 | chr12: 131913408 | 7 | | CHFR |
| | -132349534 | | | |
| 7q36.3 | chr7: 156893473- | 7 | PTPRN2§ | NCAPG2 |
| | 158821424 | | | |
| 6q16.1 | chr6: 76630464- | 76 | | FUT9, |
| | 105342994 | | | C6orf165, |
| | | | | C6orf162, |
| | | | | GJA10 |
| 5q21.1 | chr5: 85837489- | 142 | APC | APC |
| | 133480433 | | | |
| 8p11.22 | chr8: 39008109- | 7 | | C8orf4, |
| | 41238710 | | | ZMAT4 |
| 19q13.32 | chr19: 52031294- | 25 | | BBC3 |
| | 53331283 | | | |
| 10p15.3 | chr10: 1-1042949 | 4 | | TUBB8 |
| 1p31.1 | chr1: 71284749- | 4 | NEGR1§ | NEGR1 |
| | 74440273 | | | |
| 13q31.3 | chr13: 92308911- | 2 | GPC6§ | GPC6, DCT |
| | 94031607 | | | |
| 16q11.2 | chr16: 31854743- | 37 | | RBL2 |
| | 53525739 | | | |
| 20p13 | chr20: 1-325978 | 10 | | SOX12 |
| 5q35.3 | chr5: 177541057- | 43 | | SCGB3A1 |
| | 180857866 | | | |
| 1q43 | chr1: 223876038- | 173 | RYR2§ | FH, ZNF678 |
| | 247249719 | | | |
| 16p13.3 | chr16: 1-359092 | 16 | | HBZ |
| 17q21.2 | chr17: 37319013- | 22 | | CNP |
| | 37988602 | | | |
| 2p25.3 | chr2: 1-15244284 | 51 | | MYT1L |
| 3q13.31 | chr3: 116900556- | 1 | | LSAMP |
| | 120107320 | | | |
| 7q21.11 | chr7: 65877239- | 73 | MAGI2§ | CLDN4 |
| | 79629882 | | | |
| 7q35 | chr7: 144118814- | 3 | CNTNAP2§ | CNTNAP2 |
| | 148066271 | | | |
| 14q32.12 | chr14: 80741860- | 154 | | PRIMA1 |
| | 106368585 | | | |
| 16q24.3 | chr16: 88436931 - | 9 | | C16orf3 |
| | 88827254 | | | |
| 3q26.31 | chr3: 175446835- | 1 | NAALADL2§ | NAALADL2 |
| | 178263192 | | | |
| 17q25.3 | chr17: 78087533- | 8 | | ZNF750 |
| | 78774742 | | | |
| 19p12 | chr19: 21788507- | 12 | | ZNF492, |
| | 34401877 | | | ZNF99 |
| 12q23.1 | chr12: 97551177- | 3 | ANKS1B§ | ANKS1B |
| | 99047626 | | | |
| 4p16.3 | chr4: 1-435793 | 4 | | ZNF141 |
| 18p11.32 | chr18: 1-587750 | 4 | | COLEC12 |
| 2q33.2 | chr2: 204533830- | 1 | PARD3B§ | PARD3B |
| | 206266883 | | | |
| 8p21.2 | chr8: 22125332- | 63 | | DPYSL2, |
| | 30139123 | | | STMN4 |
| 8q11.22 | chr8: 42971602- | 86 | SNTG1§ | FLJ23356, |
| | 72924037 | | | ST18, |
| | | | | RB1CC1 |
| 16q23.3 | chr16: 80759878- | 2 | CDH13§ | CDH13 |
| | 82408573 | | | |
| 11q14.1 | chr11: 82612034- | 6 | DLG2§ | CCDC89, |
| | 85091467 | | | CCDC90B, |
| | | | | TMEM126A |
| 14q23.3 | chr14: 65275722- | 7 | | GPHN, |
| | 67085224 | | | MPP5 |
| 7p22.2 | chr7: 3046420- | 1 | SDK1§ | SDK1 |
| | 4279470 | | | |
| 13q34 | chr13: 111767404- | 25 | | TUBGCP3 |
| | 114142980 | | | |
| 17p12 | chr17: 10675416- | 5 | MAP2K4 | MAP2K4, |
| | 12635879 | | | ZNF18 |
| 21q22.2 | chr21: 38584860- | 19 | DSCAM§, | DSCAM |
| | 42033506 | | TMPRSS2/ | |
| | | | ERG$ | |
| 18q21.2 | chr18: 46172638- | 7 | SMAD4, | DCC |
| | 49935241 | | DCC§ | |
| 6q22.1 | chr6: 101000242- | 87 | | GTF3C6, |
| | 121511318 | | | TUBE1, |
| | | | | ROS1 |
| 14q11.2 | chr14: 1- | 140 | | ZNF219, |
| | 29140968 | | | NDRG2 |

In various embodiments, it is contemplated to use the methods identified herein to identify CNVs of segments comprising the amplified regions or genes identified in Table 5 and/or to use the methods identified herein to identify CNVs of segments comprising the deleted regions or genes identified in Table 6.

In one embodiment, the methods described herein provide a means to assess the association between gene amplification and the extent of tumor evolution. Correlation between amplification and/or deletion and stage or grade of a cancer may be prognostically important because such information may contribute to the definition of a genetically based tumor grade that would better predict the future course of disease with more advanced tumors having the worst prognosis. In addition, information about early amplification and/or deletion events may be useful in associating those events as predictors of subsequent disease progression.

Gene amplification and deletions as identified by the method can be associated with other known parameters such as tumor grade, histology, Brd/Urd labeling index, hormonal status, nodal involvement, tumor size, survival duration and other tumor properties available from epidemiological and biostatistical studies. For example, tumor DNA to be tested by the method could include atypical hyperplasia, ductal carcinoma in situ, stage I-Ill cancer and metastatic lymph nodes in order to permit the identification of associations between amplifications and deletions and stage. The associations made may make possible effective therapeutic intervention. For example, consistently amplified regions may contain an overexpressed gene, the product of which may be able to be attacked therapeutically (for example, the growth factor receptor tyrosine kinase, p185^{HER2}).

In various embodiments, the methods described herein can be used to identify amplification and/or deletion events that are associated with drug resistance by determining the copy number variation of nucleic acid sequences from primary cancers to those of cells that have metastasized to other sites. If gene amplification and/or deletion is a manifestation of karyotypic instability that allows rapid development of drug resistance, more amplification and/or deletion in primary tumors from chemoresistant patients than in tumors in chemosensitive patients would be expected. For example, if amplification of specific genes is responsible for the development of drug resistance, regions surrounding those genes would be expected to be amplified consistently in tumor cells from pleural effusions of chemoresistant patients but not in the primary tumors. Discovery of associations between gene amplification and/or deletion and the development of drug resistance may allow the identification of patients that will or will not benefit from adjuvant therapy.

In a manner similar to that described for determining the presence or absence of complete and/or partial fetal chromosomal aneuploidies in a maternal sample, methods, apparatus, and systems described herein can be used to determine the presence or absence of complete and/or partial chromosomal aneuploidies in any patient sample comprising nucleic acids e.g., DNA or cfDNA (including patient samples that are not maternal samples). The patient sample can be any biological sample type as described elsewhere herein. Preferably, the sample is obtained by non-invasive procedures. For example, the sample can be a blood sample, or the serum and plasma fractions thereof. Alternatively, the sample can be a urine sample or a fecal sample. In yet other embodiments, the sample is a tissue biopsy sample. In all cases, the sample comprises nucleic acids e.g., cfDNA or genomic DNA, which is purified, and sequenced using any of the NGS sequencing methods described previously.

Both complete and partial chromosomal aneuploidies associated with the formation, and progression of cancer can be determined according to the present method.

In various embodiments, when using the methods described herein to determine the presence and/or increased risk of cancer normalization of the data can be made with respect to the chromosome(s) for which the CNV is determined. In certain embodiments normalization of the data can be made with respect to the chromosome arm(s) for which the CNV is determined. In certain embodiments, normalization of the data can be made with respect to the particular segment(s) for which the CNV is determined.

In addition to the role of CNV in cancer, CNVs have been associated with a growing number of common complex disease, including human immunodeficiency virus (HIV), autoimmune diseases and a spectrum of neuropsychiatric disorders.

### CNVs in Infectious and Autoimmune Disease

To date a number of studies have reported association between CNV in genes involved in inflammation and the immune response and HIV, asthma, Crohn's disease and other autoimmune disorders (Fanciulli et al., Clin Genet 77:201-213 [2010]). For example, a CNV in CCL3L1, has been implicated in HIV/AIDS susceptibility (CCL3L1, 17q11.2 deletion), rheumatoid arthritis (CCL3L1, 17q11.2 deletion), and Kawasaki disease (CCL3L1, 17q11.2 duplication); a CNV in HBD-2, has been reported to predispose to colonic Crohn's disease (HDB-2, 8p23.1 deletion) and psoriasis (HDB-2, 8p23.1 deletion); a CNV in FCGR3B, was shown to predispose to glomerulonephritis in systemic lupus erthematosous (FCGR3B, 1q23 deletion, 1q23 duplication), anti-neutrophil cytoplasmic antibody (ANCA)-associated vasculatis (FCGR3B, 1q23 deletion), and increase the risk of developing rheumatoid arthritis. There are at least two inflammatory or autoimmune diseases that have been shown to be associated with CNVs at different gene loci. For example, Crohn's disease is associated with low copy number at HDB-2, but also with a common deletion polymorphism upstream of the IGRM gene that encodes a member of the p47 immunity-related GTPase family. In addition to the association with FCGR3B copy number, SLE susceptibility has also been reported to be significantly increased among subjects with a lower number of copies of complement component C4.

Associations between genomic deletions at the GSTM1 (GSTM1, 1q23deletion) and GSTT1 (GSTT1, 22q11.2 deletion) loci and increased risk of atopic asthma have been reported in a number of independent studies. In some embodiments, the methods described herein can be used to determine the presence or absence of a CNV associated with inflammation and/or autoimmune diseases. For example, the methods can be used to determine the presence of a CNV in a patient suspected to be suffering from HIV, asthma, or Crohn's disease. Examples of CNVs associated with such diseases include without limitation deletions at 17q11.2, 8p23.1, 1q23, and 22q11.2, and duplications at 17q11.2, and 1q23. In some embodiments, the present method can be used to determine the presence of CNVs in genes including but not limited to CCL3L1, HBD-2, FCGR3B, GSTM, GSTT1, C4, and IRGM.

### CNV Diseases of the Nervous System

Associations between de novo and inherited CNVs and several common neurological and psychiatric diseases have been reported in autism, schizophrenia and epilepsy, and some cases of neurodegenerative diseases such as Parkinson's disease, amyotrophic lateral sclerosis (ALS) and autosomal dominant Alzheimer's disease (Fanciulli et al., Clin Genet 77:201-213 [2010]). Cytogenetic abnormalities have been observed in patients with autism and autism spectrum disorders (ASDs) with duplications at 15q11-q13. According to the Autism Genome project Consortium, 154 CNVs including several recurrent CNVs, either on chromosome 15q11-q13 or at new genomic locations including chromosome 2p16, 1q21 and at 17p12 in a region associated with Smith-Magenis syndrome that overlaps with ASD. Recurrent microdeletions or microduplications on chromosome 16p11.2 have highlighted the observation that de novo CNVs are detected at loci for genes such as SHANK3 (22q13.3 deletion), neurexin 1 (NRXN1, 2p16.3 deletion) and the neuroglins (NLGN4, Xp22.33 deletion) that are known to regulate synaptic differentiation and regulate glutaminergic neurotransmitter release. Schizophrenia has also been associated with multiple de novo CNVs. Microdeletions and microduplications associated with schizophrenia contain an overrepresentation of genes belonging to neurodevelopmental and glutaminergic pathways, suggesting that multiple CNVs affecting these genes may contribute directly to the pathogenesis of schizophrenia e.g., ERBB4, 2q34 deletion, SLC1A3, 5p13.3 deletion; RAPEGF4, 2q31.1 deletion; CIT, 12.24 deletion; and multiple genes with de novo CNVs. CNVs have also been associated with other neurological disorders including epilepsy (CHRNA7, 15q13.3 deletion), Parkinson's disease (SNCA 4q22 duplication) and ALS (SMN1, 5q12.2.-q13.3 deletion; and SMN2 deletion). In some embodiments, the methods described herein can be used to determine the presence or absence of a CNV associated with diseases of the nervous system. For example, the methods can be used to determine the presence of a CNV in a patient suspected to be suffering from autism, schizophrenia, epilepsy, neurodegenerative diseases such as Parkinson's disease, amyotrophic lateral sclerosis (ALS) or autosomal dominant Alzheimer's disease. The methods can be used to determine CNV of genes associated with diseases of the nervous system including without limitation any of the Autism Spectrum Disorders (ASD), schizophrenia, and epilepsy, and CNV of genes associated with neurodegenerative disorders such as Parkinson's disease. Examples of CNVs associated with such diseases include without limitation duplications at 15q11-q13, 2p16, 1q21, 17p12, 16p11.2, and 4q22, and deletions at 22q13.3, 2p16.3, Xp22.33, 2q34, 5p13.3, 2q31.1, 12.24, 15q13.3, and 5q12.2. In some embodiments, the methods can be used to determine the presence of CNVs in genes including but not limited to SHANK3, NLGN4, NRXN1, ERBB4, SLC1A3, RAPGEF4, CIT, CHRNA7, SNCA, SMN1, and SMN2.

### CNV and Metabolic or Cardiovascular Diseases

The association between metabolic and cardiovascular traits, such as familial hypercholesterolemia (FH), atherosclerosis and coronary artery disease, and CNVs has been reported in a number of studies (Fanciulli et al., Clin Genet 77:201-213 [2010]). For example, germline rearrangements, mainly deletions, have been observed at the LDLR gene (LDLR, 19p13.2 deletion/duplication) in some FH patients who carry no other LDLR mutations. Another example is the LPA gene that encodes apolipoprotein(a) (apo(a)) whose plasma concentration is associated with risk of coronary artery disease, myocardial infarction (MI) and stroke Plasma concentrations of the apo(a) containing lipoprotein Lp(a) vary over 1000-fold between individuals and 90% of this variability is genetically determined at the LPA locus, with plasma concentration and Lp(a) isoform size being proportional to a highly variable number of ‘kringle 4’ repeat sequences (range 5-50). These data indicate that CNVs in at least two genes can be associated with cardiovascular risk. The methods described herein can be used in large studies to search specifically for CNV associations with cardiovascular disorders. In some embodiments, the present method can be used to determine the presence or absence of a CNV associated with metabolic or cardiovascular disease. For example, the present method can be used to determine the presence of a CNV in a patient suspected to be suffering from familial hypercholesterolemia. The methods described herein can be used to determine CNV of genes associated with metabolic or cardiovascular disease e.g., hypercholesterolemia. Examples of CNVs associated with such diseases include without limitation 19p13.2 deletion/duplication of the LDLR gene, and multiplications in the LPA gene.

### Apparatus and Systems for Determining CNV

Analysis of the sequencing data and the diagnosis derived therefrom are typically performed using various computer executed algorithms and programs. Therefore, certain embodiments employ processes involving data stored in or transferred through one or more computer systems or other processing systems. Embodiments disclosed herein also relate to apparatus for performing these operations. This apparatus may be specially constructed for the required purposes, or it may be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel. A processor or group of processors for performing the methods described herein may be of various types including microcontrollers and microprocessors such as programmable devices (e.g., CPLDs and FPGAs) and non-programmable devices such as gate array ASICs or general-purpose microprocessors.

In addition, certain embodiments relate to tangible and/or non-transitory computer readable media or computer program products that include program instructions and/or data (including data structures) for performing various computer-implemented operations. Examples of computer-readable media include, but are not limited to, semiconductor memory devices, magnetic media such as disk drives, magnetic tape, optical media such as CDs, magneto-optical media, and hardware devices that are specially configured to store and perform program instructions, such as read-only memory devices (ROM) and random-access memory (RAM). The computer readable media may be directly controlled by an end user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In various embodiments, the data or information employed in the disclosed methods and apparatus is provided in an electronic format. Such data or information may include reads and tags derived from a nucleic acid sample, counts or densities of such tags that align with particular regions of a reference sequence (e.g., that align to a chromosome or chromosome segment), reference sequences (including reference sequences providing solely or primarily polymorphisms), chromosome and segment doses, calls such as aneuploidy calls, normalized chromosome and segment values, pairs of chromosomes or segments and corresponding normalizing chromosomes or segments, counseling recommendations, diagnoses, and the like. As used herein, data or other information provided in electronic format is available for storage on a machine and transmission between machines. Typically, data in electronic format is provided digitally and may be stored as bits and/or bytes in various data structures, lists, databases, and the like. The data may be embodied electronically, optically, and the like.

One embodiment provides a computer program product for generating an output indicating the presence or absence of an aneuploidy, e.g., a fetal aneuploidy or cancer, in a test sample. The computer product may contain instructions for performing any one or more of the above-described methods for determining a chromosomal anomaly. As explained, the computer product may include a non-transitory and/or tangible computer readable medium having a computer executable or compilable logic (e.g., instructions) recorded thereon for enabling a processor to determine chromosome doses and, in some cases, whether a fetal aneuploidy is present or absent. In one example, the computer product comprises a computer readable medium having a computer executable or compilable logic (e.g., instructions) recorded thereon for enabling a processor to diagnose a fetal aneuploidy comprising: a receiving procedure for receiving sequencing data from at least a portion of nucleic acid molecules from a maternal biological sample, where said sequencing data comprises a calculated chromosome and/or segment dose; computer assisted logic for analyzing a fetal aneuploidy from said received data; and an output procedure for generating an output indicating the presence, absence or kind of said fetal aneuploidy.

The sequence information from the sample under consideration may be mapped to chromosome reference sequences to identify a number of sequence tags for each of any one or more chromosomes of interest and to identify a number of sequence tags for a normalizing segment sequence for each of said any one or more chromosomes of interest. In various embodiments, the reference sequences are stored in a database such as a relational or object database, for example.

It should be understood that it is not practical, or even possible in most cases, for an unaided human being to perform the computational operations of the methods disclosed herein. For example, mapping a single 30 bp read from a sample to any one of the human chromosomes might require years of effort without the assistance of a computational apparatus. Of course, the problem is compounded because reliable aneuploidy calls generally require mapping thousands (e.g., at least about 10,000) or even millions of reads to one or more chromosomes. In some embodiments, data sets can include from thousands to millions of sequence reads, thousands to millions of sequence tags, fragment lengths, and/or fragment length ratios, for each test sample.

The methods disclosed herein can be performed using a system for evaluation of copy number of a genetic sequence of interest in a test sample. The system comprising: (a) a sequencer for receiving nucleic acids from the test sample providing nucleic acid sequence information from the sample; (b) a processor; and (c) one or more computer-readable storage media having stored thereon instructions for execution on said processor to carry out a method for identifying any CNV, e.g., chromosomal or partial aneuploidies.

In some embodiments, the methods are instructed by a computer-readable medium having stored thereon computer-readable instructions for carrying out a method for identifying any CNV, e.g., chromosomal or partial aneuploidies. Thus, one embodiment provides a computer program product comprising one or more computer-readable non-transitory storage media having stored thereon computer-executable instructions that, when executed by one or more processors of a computer system, cause the computer system to implement a method for evaluation of copy number of a sequence of interest in a test sample comprising fetal and maternal cell-free nucleic acids. The method includes: (a) receiving sequence reads obtained by sequencing the cell-free nucleic acid fragments in the test sample; (b) aligning the sequence reads of the cell-free nucleic acid fragments or aligning fragments containing the sequence reads to bins of a reference genome comprising the sequence of interest, thereby providing test sequence tags, where the reference genome is divided into a plurality of bins; (c) measuring fragment lengths of at least some of the cell-free nucleic acid fragments present in the test sample; (d) for the sequence of interest: (i) generating a first test sequence tag quantification for fragments within a first selected fragment length range; and (ii) generating a second test sequence tag quantification for fragments within a second selected fragment length range, where the first selected fragment length range and the second selected fragment length range are different; and (e) determining the presence, absence, or no call of a copy number variation in the sequence of interest according to the first test sequence tag quantification and the second test sequence tag quantification. In some implementations, the system is configured to evaluate copy number in the test sample using the various methods and processes discussed above.

Provided in certain embodiments are systems comprising one or more microprocessors and memory, which memory comprises instructions executable by the one or more microprocessors and which memory comprises sequence reads obtained by sequencing cell-free nucleic acid fragments originating from two or more genomes in a test sample, and where the instructions executable by the one or more microprocessors are configured to: (a) align the sequence reads of the cell-free nucleic acid fragments or align fragments containing the sequence reads to bins of a reference genome comprising a sequence of interest, thereby providing test sequence tags, where the reference genome is divided into a plurality of bins; (b) measure fragment lengths of at least some of the cell-free nucleic acid fragments present in the test sample; (c) for the sequence of interest: (i) generate a first test sequence tag quantification for fragments within a first selected fragment length range; and (ii) generate a second test sequence tag quantification for fragments within a second selected fragment length range, where the first selected fragment length range and the second selected fragment length range are different; and (d) determine a presence, absence, or no call of a copy number variation in the sequence of interest according to the first test sequence tag quantification and the second test sequence tag quantification.

Also provided in certain embodiments are machines comprising one or more microprocessors and memory, which memory comprises instructions executable by the one or more microprocessors and which memory comprises sequence reads obtained by sequencing cell-free nucleic acid fragments originating from two or more genomes in a test sample, and where the instructions executable by the one or more microprocessors are configured to: (a) align the sequence reads of the cell-free nucleic acid fragments or align fragments containing the sequence reads to bins of a reference genome comprising a sequence of interest, thereby providing test sequence tags, where the reference genome is divided into a plurality of bins; (b) measure fragment lengths of at least some of the cell-free nucleic acid fragments present in the test sample; (c) for the sequence of interest: (i) generate a first test sequence tag quantification for fragments within a first selected fragment length range; and (ii) generate a second test sequence tag quantification for fragments within a second selected fragment length range, where the first selected fragment length range and the second selected fragment length range are different; and (d) determine a presence, absence, or no call of a copy number variation in the sequence of interest according to the first test sequence tag quantification and the second test sequence tag quantification.

Also provided in certain embodiments are non-transitory computer-readable storage media with an executable program stored thereon, where the program instructs a microprocessor to perform the following: (a) access sequence reads obtained by sequencing cell-free nucleic acid fragments originating from two or more genomes in a test sample; (b) align the sequence reads of the cell-free nucleic acid fragments or align fragments containing the sequence reads to bins of a reference genome comprising a sequence of interest, thereby providing test sequence tags, where the reference genome is divided into a plurality of bins; (c) measure fragment lengths of at least some of the cell-free nucleic acid fragments present in the test sample; (d) for the sequence of interest: (i) generate a first test sequence tag quantification for fragments within a first selected fragment length range; and (ii) generate a second test sequence tag quantification for fragments within a second selected fragment length range, where the first selected fragment length range and the second selected fragment length range are different; and (e) determine a presence, absence, or no call of a copy number variation in the sequence of interest according to the first test sequence tag quantification and the second test sequence tag quantification.

In some embodiments, instructions may further include automatically recording information pertinent to the method such as chromosome doses and the presence or absence of a CNV and/or a fetal chromosomal aneuploidy in a patient medical record for a human subject providing the test sample or maternal test sample. The patient medical record may be maintained by, for example, a laboratory, physician's office, a hospital, a health maintenance organization, an insurance company, or a personal medical record website. Further, based on the results of the processor-implemented analysis, the method may further involve prescribing, initiating, and/or altering treatment of a human subject from whom the test sample or maternal test sample was taken. This may involve performing one or more additional tests or analyses on additional samples taken from the subject.

Disclosed methods can also be performed using a computer processing system which is adapted or configured to perform a method for identifying any CNV, e.g., chromosomal or partial aneuploidies. One embodiment provides a computer processing system which is adapted or configured to perform a method as described herein. In one embodiment, the apparatus comprises a sequencing device adapted or configured for sequencing at least a portion of the nucleic acid molecules in a sample to obtain the type of sequence information described elsewhere herein. The apparatus may also include components for processing the sample. Such components are described elsewhere herein.

Sequence or other data, can be input into a computer or stored on a computer readable medium either directly or indirectly. In one embodiment, a computer system is directly coupled to a sequencing device that reads and/or analyzes sequences of nucleic acids from samples. Sequences or other information from such tools are provided via interface in the computer system. Alternatively, the sequences processed by system are provided from a sequence storage source such as a database or other repository. Once available to the processing apparatus, a memory device or mass storage device buffers or stores, at least temporarily, sequences of the nucleic acids. In addition, the memory device may store tag counts for various chromosomes or genomes, and the like. The memory may also store various routines and/or programs for analyzing the presenting the sequence or mapped data. Such programs/routines may include programs for performing statistical analyses, and the like.

In one example, a user provides a sample into a sequencing apparatus. Data is collected and/or analyzed by the sequencing apparatus which is connected to a computer. Software on the computer allows for data collection and/or analysis. Data can be stored, displayed (via a monitor or other similar device), and/or sent to another location. The computer may be connected to the internet which is used to transmit data to a handheld device utilized by a remote user (e.g., a physician, scientist or analyst). It is understood that the data can be stored and/or analyzed prior to transmittal. In some embodiments, raw data is collected and sent to a remote user or apparatus that will analyze and/or store the data. Transmittal can occur via the internet, but can also occur via satellite or other connection. Alternately, data can be stored on a computer-readable medium and the medium can be shipped to an end user (e.g., via mail). The remote user can be in the same or a different geographical location including, but not limited to a building, city, state, country or continent.

In some embodiments, the methods also include collecting data regarding a plurality of polynucleotide sequences (e.g., reads, tags and/or reference chromosome sequences) and sending the data to a computer or other computational system. For example, the computer can be connected to laboratory equipment, e.g., a sample collection apparatus, a nucleotide amplification apparatus, a nucleotide sequencing apparatus, or a hybridization apparatus. The computer can then collect applicable data gathered by the laboratory device. The data can be stored on a computer at any step, e.g., while collected in real time, prior to the sending, during or in conjunction with the sending, or following the sending. The data can be stored on a computer-readable medium that can be extracted from the computer. The data collected or stored can be transmitted from the computer to a remote location, e.g., via a local network or a wide area network such as the internet. At the remote location various operations can be performed on the transmitted data as described below.

Among the types of electronically formatted data that may be stored, transmitted, analyzed, and/or manipulated in systems, apparatus, and methods disclosed herein are the following:
Reads obtained by sequencing nucleic acids in a test sample
Tags obtained by aligning reads to a reference genome or other reference sequence or sequences
The reference genome or sequence
Fragment length measurements
Fragment length ratios
First test sequence tag quantification for fragments within a first selected fragment length range (e.g., for a sequence of interest)
Second test sequence tag quantification for fragments within a second selected fragment length range (e.g., for a sequence of interest)
Numbers of sequence tags aligning to one or more bins (e.g., in a sequence of interest)
Normalized numbers of sequence tags aligning to one or more bins (e.g., in a sequence of interest)
Fetal fraction and/or tumor fraction
Sequence tag density—Counts or numbers of tags for each of two or more regions (typically chromosomes, chromosome segments, sequence of interest) of a reference genome or other reference sequences
Identities of normalizing chromosomes or chromosome segments for particular chromosomes or sequence of interest
Doses for chromosomes or chromosome segments (or other regions) obtained from chromosomes or sequences of interest and corresponding normalizing chromosomes or segments
Thresholds for calling chromosome doses as either affected, non-affected, or no call
The actual calls of chromosome doses
Diagnoses (clinical condition associated with the calls)
Recommendations for further tests derived from the calls and/or diagnoses
Treatment and/or monitoring plans derived from the calls and/or diagnoses

These various types of data may be obtained, stored transmitted, analyzed, and/or manipulated at one or more locations using distinct apparatus. The processing options span a wide spectrum. At one end of the spectrum, all or much of this information is stored and used at the location where the test sample is processed, e.g., a doctor's office or other clinical setting. In other extreme, the sample is obtained at one location, it is processed and optionally sequenced at a different location, reads are aligned and calls are made at one or more different locations, and diagnoses, recommendations, and/or plans are prepared at still another location (which may be a location where the sample was obtained).

In various embodiments, the reads are generated with the sequencing apparatus and then transmitted to a remote site where they are processed to produce aneuploidy calls. At this remote location, as an example, the reads are aligned to a reference sequence to produce tags, which are counted and assigned to chromosomes or segments of interest. Also at the remote location, the counts are converted to doses using associated normalizing chromosomes or segments. Still further, at the remote location, the doses are used to generate aneuploidy calls.

Among the processing operations that may be employed at distinct locations are the following:
Sample collection
Sample processing preliminary to sequencing
Sequencing
Analyzing sequence data and deriving aneuploidy calls
Diagnosis
Reporting a diagnosis and/or a call to patient or health care provider
Developing a plan for further treatment, testing, and/or monitoring
Executing the plan
Counseling

Any one or more of these operations may be automated as described elsewhere herein. Typically, the sequencing and the analyzing of sequence data and deriving aneuploidy calls will be performed computationally. The other operations may be performed manually or automatically.

Examples of locations where sample collection may be performed include health practitioners' offices, clinics, patients' homes (where a sample collection tool or kit is provided), and mobile health care vehicles. Examples of locations where sample processing prior to sequencing may be performed include health practitioners' offices, clinics, patients' homes (where a sample processing apparatus or kit is provided), mobile health care vehicles, and facilities of aneuploidy analysis providers. Examples of locations where sequencing may be performed include health practitioners' offices, clinics, health practitioners' offices, clinics, patients' homes (where a sample sequencing apparatus and/or kit is provided), mobile health care vehicles, and facilities of aneuploidy analysis providers. The location where the sequencing takes place may be provided with a dedicated network connection for transmitting sequence data (typically reads) in an electronic format. Such connection may be wired or wireless and have and may be configured to send the data to a site where the data can be processed and/or aggregated prior to transmission to a processing site. Data aggregators can be maintained by health organizations such as Health Maintenance Organizations (HMOs).

The analyzing and/or deriving operations may be performed at any of the foregoing locations or alternatively at a further remote site dedicated to computation and/or the service of analyzing nucleic acid sequence data. Such locations include for example, clusters such as general-purpose server farms, the facilities of an aneuploidy analysis service business, and the like. In some embodiments, the computational apparatus employed to perform the analysis is leased or rented. The computational resources may be part of an internet accessible collection of processors such as processing resources colloquially known as the cloud. In some cases, the computations are performed by a parallel or massively parallel group of processors that are affiliated or unaffiliated with one another. The processing may be accomplished using distributed processing such as cluster computing, grid computing, and the like. In such embodiments, a cluster or grid of computational resources collective form a super virtual computer composed of multiple processors or computers acting together to perform the analysis and/or derivation described herein. These technologies as well as supercomputers may be employed to process sequence data as described herein. Each is a form of parallel computing that relies on processors or computers. In the case of grid computing these processors (often whole computers) are connected by a network (private, public, or the Internet) using a protocol such as Ethernet. By contrast, a supercomputer has many processors connected by a local high-speed computer bus.

In certain embodiments, the diagnosis (e.g., the fetus has Downs syndrome or the patient has a particular type of cancer) is generated at the same location as the analyzing operation. In other embodiments, it is performed at a different location. In some examples, reporting the diagnosis is performed at the location where the sample was taken, although this need not be the case. Examples of locations where the diagnosis can be generated or reported and/or where developing a plan is performed include health practitioners' offices, clinics, internet sites accessible by computers, and handheld devices such as cell phones, tablets, smart phones, and the like having a wired or wireless connection to a network. Examples of locations where counseling is performed include health practitioners' offices, clinics, internet sites accessible by computers, handheld devices, and the like.

In some embodiments, the sample collection, sample processing, and sequencing operations are performed at a first location and the analyzing and deriving operation is performed at a second location. However, in some cases, the sample collection is collected at one location (e.g., a health practitioner's office or clinic) and the sample processing and sequencing is performed at a different location that is optionally the same location where the analyzing and deriving take place.

In various embodiments, a sequence of the above-listed operations may be triggered by a user or entity initiating sample collection, sample processing and/or sequencing. After one or more these operations have begun execution, the other operations may naturally follow. For example, the sequencing operation may cause reads to be automatically collected and sent to a processing apparatus which then conducts, often automatically and possibly without further user intervention, the sequence analysis and derivation of aneuploidy operation. In some implementations, the result of this processing operation is then automatically delivered, possibly with reformatting as a diagnosis, to a system component or entity that reports the information to a health professional and/or patient. As explained such information can also be automatically processed to produce a treatment, testing, and/or monitoring plan, possibly along with counseling information. Thus, initiating an early-stage operation can trigger an end-to-end sequence in which the health professional, patient or other concerned party is provided with a diagnosis, a plan, counseling and/or other information useful for acting on a physical condition. This is accomplished even though parts of the overall system are physically separated and possibly remote from the location of, e.g., the sample and sequence apparatus.

An example implementation of a dispersed system for producing a call or diagnosis from a test sample is described as follows. A sample collection location is used for obtaining a test sample from a patient such as a pregnant female or a putative cancer patient. The samples are then provided to a processing and sequencing location where the test sample may be processed and sequenced as described above. Such location includes apparatus for processing the sample as well as apparatus for sequencing the processed sample. The result of the sequencing, as described elsewhere herein, is a collection of reads which are typically provided in an electronic format and provided to a network such as the Internet.

The sequence data is provided to a remote location where analysis and call generation are performed. This location may include one or more powerful computational devices such as computers or processors. After the computational resources at this location have completed their analysis and generated a call from the sequence information received, the call is relayed back to the network. In some implementations, not only is a call generated at the remote location but an associated diagnosis is also generated. The call and/or diagnosis are then transmitted across the network and back to the sample collection location. As explained, this is simply one of many variations on how the various operations associated with generating a call or diagnosis may be divided among various locations. One common variant involves providing sample collection and processing and sequencing in a single location. Another variation involves providing processing and sequencing at the same location as analysis and call generation. In the most granular sense, each of the following operations is performed at a separate location: sample collection, sample processing, sequencing, read alignment, calling, diagnosis, and reporting and/or plan development.

In one embodiment that aggregates some of these operations, sample processing and sequencing are performed in one location and read alignment, calling, and diagnosis are performed at a separate location. In another implementation, sample collection, sample processing, and sequencing are all performed at the same location. In this implementation, read alignment and calling are performed in a second location. Finally, diagnosis and reporting and/or plan development are performed in a third location. In one implementation, sample collection is performed at a first location, sample processing, sequencing, read alignment, calling, and diagnosis are all performed together at a second location, and reporting and/or plan development are performed at a third location. Finally, in another implementation, sample collection is performed at a first location, sample processing, sequencing, read alignment, and calling are all performed at a second location, and diagnosis and reporting and/or plan management are performed at a third location.

One embodiment provides a system for use in determining the presence or absence of any one or more different complete fetal chromosomal aneuploidies in a maternal test sample comprising fetal and maternal nucleic acids, the system including a sequencer for receiving a nucleic acid sample and providing fetal and maternal nucleic acid sequence information from the sample; a processor; and a machine readable storage medium comprising instructions for execution on said processor, where the instructions comprise code for performing one or more workflow components described herein.

In some embodiments, the instructions comprise code for calculating a single chromosome dose for each of the any one or more chromosomes of interest. In some embodiments, the instructions comprise code for calculating a chromosome dose for a selected one of the chromosomes of interest as the ratio of the number of sequence tags identified for the selected chromosome of interest and the number of sequence tags identified for a corresponding at least one normalizing chromosome sequence or normalizing chromosome segment sequence for the selected chromosome of interest.

In some embodiments, the system further comprises code for repeating the calculating of a chromosome dose for each of any remaining chromosome segments of the any one or more segments of any one or more chromosomes of interest.

In some embodiments, the one or more chromosomes of interest selected from chromosomes 1-22, X, and Y comprise at least twenty chromosomes selected from chromosomes 1-22, X, and Y, and where the instructions comprise instructions for determining the presence or absence of at least twenty different complete fetal chromosomal aneuploidies is determined.

In some embodiments, the at least one normalizing chromosome sequence is a group of chromosomes selected from chromosomes 1-22, X, and Y. In other embodiments, the at least one normalizing chromosome sequence is a single chromosome selected from chromosomes 1-22, X, and Y.

Another embodiment provides a system for use in determining the presence or absence of any one or more different partial fetal chromosomal aneuploidies in a maternal test sample comprising fetal and maternal nucleic acids, the system comprising: a sequencer for receiving a nucleic acid sample and providing fetal and maternal nucleic acid sequence information from the sample; a processor; and a machine readable storage medium comprising instructions for execution on said processor, where the instructions comprise code for performing one or more workflow components described herein.

In some embodiments, the instructions comprise code for calculating a single chromosome segment dose. In some embodiments, the instructions comprise code for calculating a chromosome segment dose for a selected one of the chromosome segments as the ratio of the number of sequence tags identified for the selected chromosome segment and the number of sequence tags identified for a corresponding normalizing segment sequence for the selected chromosome segment.

In some embodiments, the system further comprises code for repeating the calculating of a chromosome segment dose for each of any remaining chromosome segments of the any one or more segments of any one or more chromosomes of interest.

In some embodiments, the system further comprises (i) code for repeating certain workflow steps for test samples from different maternal subjects, and (ii) code for determining the presence or absence of any one or more different partial fetal chromosomal aneuploidies in each of said samples.

In other embodiments of any of the systems provided herein, the code further comprises code for automatically recording the presence or absence of a fetal chromosomal aneuploidy in a patient medical record for a human subject providing the maternal test sample, where the recording is performed using the processor.

In some embodiments of any of the systems provided herein, the sequencer is configured to perform next generation sequencing (NGS). In some embodiments, the sequencer is configured to perform massively parallel sequencing. In some embodiments, the sequencer is configured to perform non-targeted massively parallel sequencing. In some embodiments, the sequencer is configured to perform massively parallel sequencing using sequencing-by-synthesis with reversible dye terminators. In other embodiments, the sequencer is configured to perform sequencing-by-ligation. In yet other embodiments, the sequencer is configured to perform single molecule sequencing.

### Certain Implementations

Following are non-limiting examples of certain implementations of the technology.

A1. A method for determining presence, absence, or no call of a copy number variation (CNV) of a nucleic acid sequence of interest in a test sample comprising cell-free nucleic acid fragments originating from two or more genomes, the method comprising:
(a) receiving sequence reads obtained by sequencing the cell-free nucleic acid fragments in the test sample;
(b) aligning the sequence reads of the cell-free nucleic acid fragments or aligning fragments containing the sequence reads to bins of a reference genome comprising the sequence of interest, thereby providing test sequence tags, wherein the reference genome is divided into a plurality of bins;
(c) measuring fragment lengths of at least some of the cell-free nucleic acid fragments present in the test sample;
(d) for the sequence of interest:
   (i) generating a first test sequence tag quantification for fragments within a first selected fragment length range; and
   (ii) generating a second test sequence tag quantification for fragments within a second selected fragment length range, wherein the first selected fragment length range and the second selected fragment length range are different; and
(e) determining the presence, absence, or no call of a copy number variation in the sequence of interest according to the first test sequence tag quantification and the second test sequence tag quantification.

A2. The method of embodiment A1, wherein the copy number variation (CNV) is a chromosome aneuploidy.

A3. The method of embodiment A1, wherein the copy number variation (CNV) is a partial chromosome aneuploidy.

A4. The method of embodiment A1, wherein the copy number variation (CNV) is a microduplication or microdeletion.

A5. The method of any one of embodiments A1-A4, wherein the sequence of interest comprises a chromosome.

A6. The method of any one of embodiments A1-A4, wherein the sequence of interest comprises part a chromosome.

A7. The method of any one of embodiments A1-A4, wherein the sequence of interest comprises a region tested for presence, absence, or no call of a microduplication or microdeletion.

A8. The method of any one of embodiments A1-A7, wherein the two or more genomes comprise a maternal genome and a fetal genome.

A9. The method of embodiment A8, wherein the test sample is from a pregnant subject.

A10. The method of any one of embodiments A1-A47 wherein the two or more genomes comprise a host genome and a tumor genome.

A11. The method of embodiment A10, wherein the test sample is from a cancer patient.

A12. The method of any one of embodiments A1-A11, wherein the sequence reads are obtained by a paired-end sequencing process and the sequence reads are paired-end sequence reads.

A13. The method of embodiment A12, wherein the fragment lengths are measured in (c) according to sequence tag positions of the paired-end sequence reads.

A14. The method of any one of embodiments A1-A13, wherein generating the first test sequence tag quantification comprises determining a number of sequence tags aligning to each bin in the sequence of interest, wherein the sequence tags are from fragments within the first selected fragment length range.

A15. The method of embodiment A14, wherein the first test sequence tag quantification comprises a measure of central tendency for the numbers of sequence tags aligning to the bins in the sequence of interest.

A16. The method of embodiment A15, wherein the measure of central tendency is a mean.

A17. The method of embodiment A14, wherein generating the first test sequence tag quantification further comprises normalizing the number of sequence tags aligning to each bin by accounting for bin-to-bin variations due to factors other than copy number variation, thereby generating a normalized number of sequence tags aligning to each bin.

A18. The method of embodiment A17, wherein the first test sequence tag quantification comprises a measure of central tendency for the normalized numbers of sequence tags aligning to the bins in the sequence of interest.

A19. The method of embodiment A18, wherein the measure of central tendency is a mean.

A20. The method of any one of embodiments A14-A19, wherein the first test sequence tag quantification comprises a shift from a fixed value.

A21. The method of embodiment A20, wherein the fixed value is 1.

A22. The method of embodiment A20 or A21, wherein the first test sequence tag quantification comprises an absolute value of a shift from a fixed value.

A23. The method of embodiment A22, wherein the first test sequence tag quantification is determined according to the following:

|1−μ_{first}|

wherein μ_{first} is the mean of normalized numbers of sequence tags aligning to the bins in the sequence of interest, wherein the sequence tags are from fragments within the first selected fragment length range.

A24. The method of any one of embodiments A1-A33, wherein generating the second test sequence tag quantification comprises determining a number of sequence tags aligning to each bin in the sequence of interest, wherein the sequence tags are from fragments within the second selected fragment length range.

A25. The method of embodiment A24, wherein the second test sequence tag quantification comprises a measure of central tendency for the numbers of sequence tags aligning to the bins in the sequence of interest.

A26. The method of embodiment A25, wherein the measure of central tendency is a mean.

A27. The method of embodiment A24, wherein generating the second test sequence tag quantification further comprises normalizing the number of sequence tags aligning to each bin by accounting for bin-to-bin variations due to factors other than copy number variation, thereby generating a normalized number of sequence tags aligning to each bin.

A28. The method of embodiment A27, wherein the second test sequence tag quantification comprises a measure of central tendency for the normalized numbers of sequence tags aligning to the bins in the sequence of interest.

A29. The method of embodiment A28, wherein the measure of central tendency is a mean.

A30. The method of any one of embodiments A24-A29, wherein the second test sequence tag quantification comprises a shift from a fixed value.

A31. The method of embodiment A30, wherein the fixed value is 1.

A32. The method of embodiment A30 or A31, wherein the second test sequence tag quantification comprises an absolute value of a shift from a fixed value.

A33. The method of embodiment A22, wherein the second test sequence tag quantification is determined according to the following:

|1−μ_{second}|

wherein μ_{second} is the mean of normalized numbers of sequence tags aligning to the bins in the sequence of interest, wherein the sequence tags are from fragments within the second selected fragment length range.

A34. The method of any one of embodiments A1-A33, wherein the first selected fragment length range is about 80 bases to about 150 bases.

A35. The method of any one of embodiments A1-A34, wherein the second selected fragment length range is about 50 bases to about 300 bases.

A36. The method of any one of embodiments A1-A35, further comprising determining a ratio value of the first test sequence tag quantification to the second test sequence tag quantification.

A37. The method of embodiment A36, wherein the ratio value is determined according to the following:

$$ratio value = \frac{\vert 1 - {μ}_{first} \vert}{\vert 1 - {μ}_{second} \vert}$$

wherein:
μ_{first} is the mean of normalized numbers of sequence tags aligning to the bins in the sequence of interest, wherein the sequence tags are from fragments within the first selected fragment length range; and
μ_{second} is the mean of normalized numbers of sequence tags aligning to the bins in the sequence of interest, wherein the sequence tags are from fragments within the second selected fragment length range.

A37. The method of embodiment A36 or A37, wherein determining the presence, absence, or no call of the copy number variation in the sequence of interest in (e) is according to the ratio value of the first test sequence tag quantification to the second test sequence tag quantification.

A38. The method of embodiment A37, wherein the presence, absence, or no call of the copy number variation is determined according to a ratio value threshold.

A39. The method of embodiment A38, wherein the ratio value threshold is about 1.2.

A40. The method of embodiment A39, comprising determining the presence of the copy number variation based, at least in part, on a determination that, for the test sample, the ratio value is greater than or equal to 1.2.

A41. The method of embodiment A39, comprising determining the absence or no call of the copy number variation based, at least in part, on a determination that, for the test sample, the ratio value is less than 1.2.

A42. The method of any one of embodiments A1-A41, further comprising prior to (a), sequencing the cell-free nucleic acid fragments in the test sample by a sequencing process.

A43. The method of embodiment A42, wherein the sequencing process is a non-targeted sequencing process.

A44. The method of embodiment A42, wherein the sequencing process is a massively parallel sequencing process.

A45. The method of embodiment A42, wherein the sequencing process is a non-targeted massively parallel sequencing process.

A46. The method of any one of embodiments A42-A45, wherein the circulating cell-free (CCF) nucleic acid from the test sample is sequenced at a fold coverage of 1.0 or greater.

A47. The method of any one of embodiments A42-A45, wherein the circulating cell-free (CCF) nucleic acid from the test sample is sequenced at a fold coverage of less than 1.0.

A48. The method of any one of embodiments A42-A47, wherein the sequencing process generates thousands to millions of sequence reads.

A49. The method of any one of embodiments A1-A48, further comprising determining a fraction of fragments originating from a first genome in the two or more genomes for the test sample.

A50. The method of embodiment A49, wherein the fraction of fragments originating from the first genome is a fetal fraction.

A51. The method of embodiment A49, wherein the fraction of fragments originating from the first genome is a tumor fraction.

A52. The method of any one of embodiments A49-A51, wherein the fraction of fragments originating from the first genome is determined according to the first test sequence tag quantification for fragments within the first selected fragment length range.

A53. The method of embodiment A52, wherein the fraction of fragments originating from the first genome corresponds to an absolute value of a shift of the first test sequence tag quantification from a fixed value.

A54. The method of embodiment A52, wherein the fraction of fragments originating from the first genome corresponds to an absolute value of a shift of a normalized first test sequence tag quantification from a fixed value.

A55. The method of embodiment A53 or A54, wherein the fixed value is 1.

A56. The method of any one of embodiments A1-A55, further comprising determining whether one or both genomes in the two or more genomes for the test sample comprise the CNV.

A57. The method of embodiment A56, wherein a determination that both genomes comprise the CNV, is based, in part, on a ratio of the first test sequence tag quantification to the second test sequence tag quantification, wherein the ratio value is about 1.

A58. The method of any one of embodiments A1-A57, further comprising determining presence or absence of a mosaicism for a genome in the two or more genomes for the test sample.

A59. The method of any one of embodiments A1-A58, further comprising determining a level of a mosaicism for a genome in the two or more genomes for the test sample.

A60. The method of embodiment A58 or A59, wherein the mosaicism is a maternal mosaicism.

A61. The method of embodiment A60, wherein the presence, absence, and/or level of maternal mosaicism is determined, in part, according to the second test sequence tag quantification for fragments within the second selected fragment length range.

A62. The method of embodiment A61, wherein the presence, absence, and/or level of maternal mosaicism is determined, in part, according to an absolute value of a shift of the second test sequence tag quantification from a fixed value.

A63. The method of embodiment A62, wherein the presence, absence, and/or level of maternal mosaicism is determined, in part, according to an absolute value of a shift of a normalized second test sequence tag quantification from a fixed value.

A64. The method of embodiment A61 or A62, wherein the fixed value is 1.

B1. A system comprising one or more microprocessors and memory, which memory comprises instructions executable by the one or more microprocessors and which memory comprises sequence reads obtained by sequencing cell-free nucleic acid fragments originating from two or more genomes in a test sample, and wherein the instructions executable by the one or more microprocessors are configured to:
(a) align the sequence reads of the cell-free nucleic acid fragments or align fragments containing the sequence reads to bins of a reference genome comprising a sequence of interest, thereby providing test sequence tags, wherein the reference genome is divided into a plurality of bins;
(b) measure fragment lengths of at least some of the cell-free nucleic acid fragments present in the test sample;
(c) for the sequence of interest:
   (i) generate a first test sequence tag quantification for fragments within a first selected fragment length range; and
   (ii) generate a second test sequence tag quantification for fragments within a second selected fragment length range, wherein the first selected fragment length range and the second selected fragment length range are different; and
(d) determine a presence, absence, or no call of a copy number variation in the sequence of interest according to the first test sequence tag quantification and the second test sequence tag quantification.

B2. The system of embodiment B1 further comprising one or more features of embodiments A2-A64 and/or further configured to perform one or more methods of embodiments A2-A64.

C1. A machine comprising one or more microprocessors and memory, which memory comprises instructions executable by the one or more microprocessors and which memory comprises sequence reads obtained by sequencing cell-free nucleic acid fragments originating from two or more genomes in a test sample, and wherein the instructions executable by the one or more microprocessors are configured to:
(a) align the sequence reads of the cell-free nucleic acid fragments or align fragments containing the sequence reads to bins of a reference genome comprising a sequence of interest, thereby providing test sequence tags, wherein the reference genome is divided into a plurality of bins;
(b) measure fragment lengths of at least some of the cell-free nucleic acid fragments present in the test sample;
(c) for the sequence of interest:
   (i) generate a first test sequence tag quantification for fragments within a first selected fragment length range; and
   (ii) generate a second test sequence tag quantification for fragments within a second selected fragment length range, wherein the first selected fragment length range and the second selected fragment length range are different; and
(d) determine a presence, absence, or no call of a copy number variation in the sequence of interest according to the first test sequence tag quantification and the second test sequence tag quantification.

C2. The machine of embodiment C1 further comprising one or more features of embodiments A2-A64 and/or further configured to perform one or more methods of embodiments A2-A64.

D1. A non-transitory computer-readable storage medium with an executable program stored thereon, where the program instructs a microprocessor to perform the following:
(a) access sequence reads obtained by sequencing cell-free nucleic acid fragments originating from two or more genomes in a test sample;
(b) align the sequence reads of the cell-free nucleic acid fragments or align fragments containing the sequence reads to bins of a reference genome comprising a sequence of interest, thereby providing test sequence tags, wherein the reference genome is divided into a plurality of bins;
(c) measure fragment lengths of at least some of the cell-free nucleic acid fragments present in the test sample;
(d) for the sequence of interest:
   (i) generate a first test sequence tag quantification for fragments within a first selected fragment length range; and
   (ii) generate a second test sequence tag quantification for fragments within a second selected fragment length range, wherein the first selected fragment length range and the second selected fragment length range are different; and
(e) determine a presence, absence, or no call of a copy number variation in the sequence of interest according to the first test sequence tag quantification and the second test sequence tag quantification.

D2. The non-transitory computer-readable storage medium of embodiment D1 further comprising one or more features of embodiments A2-A64 and/or further configured to perform one or more methods of embodiments A2-A64.

E1. A method for determining presence, absence, or no call of a copy number variation (CNV) of a nucleic acid sequence of interest in a test sample from a pregnant subject comprising cell-free nucleic acid fragments originating from two or more genomes, wherein the two or more genomes comprise a maternal genome and a fetal genome, the method comprising:
(a) receiving sequence reads obtained by sequencing the cell-free nucleic acid fragments in the test sample, wherein the sequence reads are obtained by a paired-end sequencing process and the sequence reads are paired-end sequence reads;
(b) aligning the sequence reads of the cell-free nucleic acid fragments or aligning fragments containing the sequence reads to bins of a reference genome comprising the sequence of interest, thereby providing test sequence tags, wherein the reference genome is divided into a plurality of bins;
(c) measuring fragment lengths of at least some of the cell-free nucleic acid fragments present in the test sample according to sequence tag positions of the paired-end sequence reads;
(d) for the sequence of interest:
   (i) generating a first test sequence tag quantification for fragments within a first selected fragment length range, wherein the first test sequence tag quantification comprises a mean of normalized numbers of sequence tags aligning to the bins in the sequence of interest and an absolute value of a shift from a fixed value of 1; and
   (ii) generating a second test sequence tag quantification for fragments within a second selected fragment length range, wherein the second test sequence tag quantification comprises a mean of normalized numbers of sequence tags aligning to the bins in the sequence of interest and an absolute value of a shift from a fixed value of 1, wherein the first selected fragment length range is about 80 bases to about 150 bases and the second selected fragment length range is about 50 bases to about 300 bases;
(e) generating a ratio value of the first test sequence tag quantification to the second test sequence tag quantification; and
(f) determining the presence, absence, or no call of a copy number variation in the sequence of interest according to a ratio value threshold, wherein the ratio value threshold is 1.2.

### EXAMPLES

The examples set forth below illustrate certain implementations and do not limit the technology.

### Example 1: cfDNA Size Selection and VICQC

Non-invasive prenatal testing (NIPT) detects fetal copy number events from a blood draw from a pregnant subject, where the sample comprises approximately 90% maternal cell-free DNA and approximately 10% fetal cell-free DNA. Such tests typically rely on identifying regions of the genome with significant differences in coverage compared to the rest of the genome, and assumes the mother does not contribute any copy number events (such events would likely result in a detectable phenotype prior to the test). Not all events may result in a phenotype, however, and a solution is needed to discriminate between maternal and fetal events in the sample in order to exclusively report fetal-related copy number events.

Fetal cell-free DNA fragments typically are slightly shorter than maternal cell-free DNA fragments, and previous NIPT products have leveraged analyzing only shorter DNA fragments to enrich for a fetal signal. This Example describes a bioinformatics technique comparing relative signals in short fragments vs. all fragments in order to categorize events as maternal or fetal in origin and only report fetal events to the customer. The technique is based, in part, on a prediction that a fetal event would have an increased signal in shorter fragments relative to all available fragments whereas a maternal event would not.

Previous NIPT pipelines relied on utilizing a z-score to detect significant changes in coverage for a given chromosome. To improve specificity and reduce false positives, this process was further refined by adding a secondary log likelihood ratio (LLR) estimate which compared the t-statistic signal detected to the estimated fetal fraction of the sample. A bivariate LLR score was selected, separating reads by fragment length (short vs. long) in order to boost signal. This additional step helped remove samples which had a sufficient z-score but the signal observed did not trend with the expected signal given the fetal fraction. The bivariate LLR estimate, however, only removes samples with a lower signal than expected. To separate events of maternal and fetal origin, the ratio of short vs. long reads can be estimated and substituted in the t-statistic signal calculation. A univariate LLR is subsequently calculated, and only events with an enrichment in short reads will result in a positive LLR score. This is equivalent to normalizing the data according to maternal signal. If the signal is fetal in origin, this normalization will increase magnitude of the event as size selection favors fetal DNA. If the signal is maternal in origin, this normalization will not change the magnitude of the event.

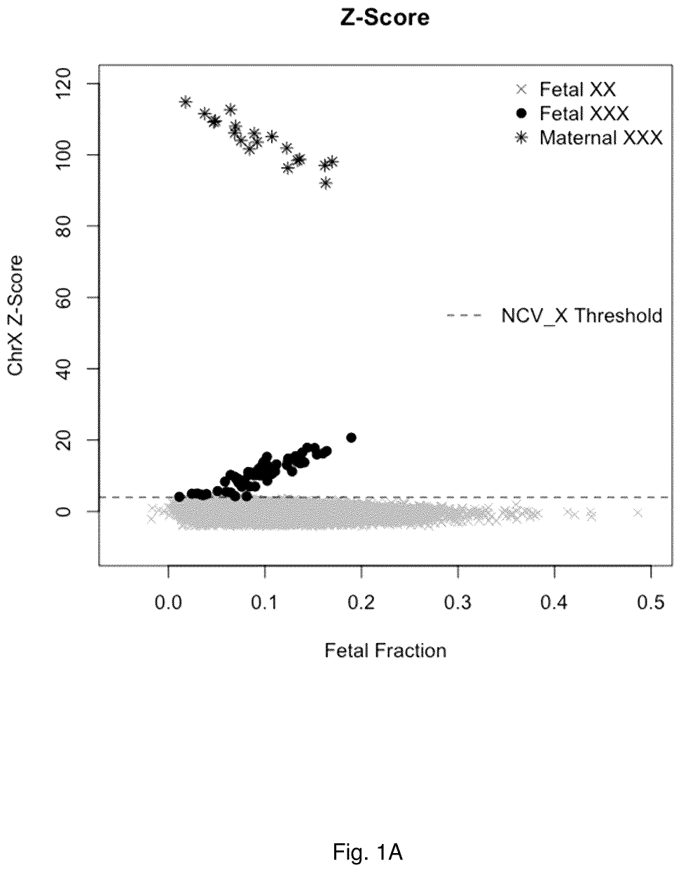

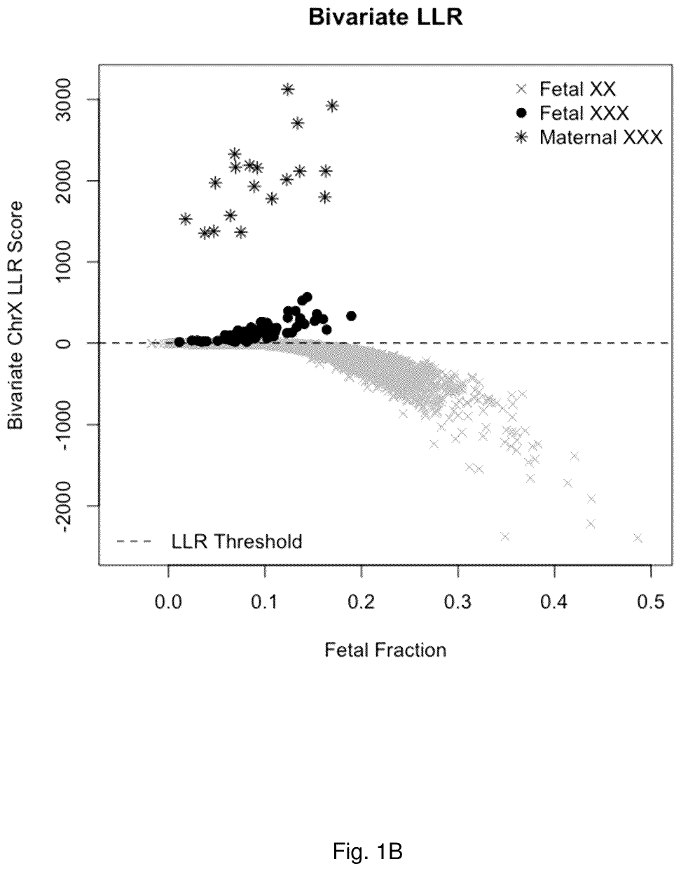

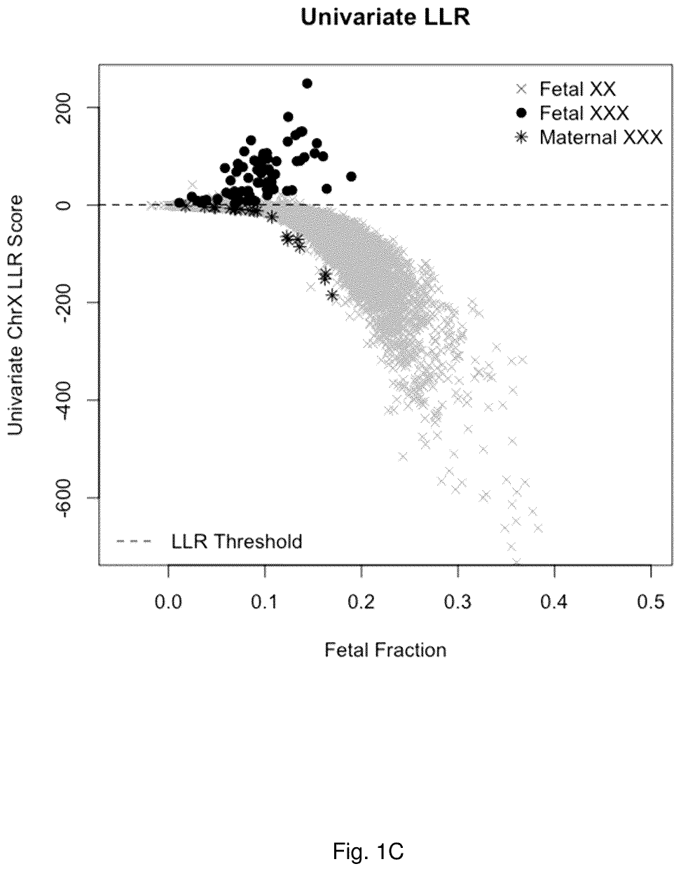

An example of this approach is provided in FIGS. 1A-1C. Chromosome X is one of the most common regions impacted by maternal events, as XXX is the most common female chromosome abnormality (1 in 1000 female births) and only typically diagnosed 10% of the time. FIG. 1A and FIG. 1B demonstrate the relationship observed between fetal fraction and the z-score or bivariate LLR, respectively, with the dashed line corresponding to the thresholds currently applied in the NIPT pipeline for each metric. Both approaches would result in classifying the sample as “fetal XXX”, and result in a subset of false positive calls (maternal XXX datapoints). When the univariate LLR is applied, the maternal XXX samples drop out and fall below the threshold (FIG. 1C). This approach also can be applied to detect maternal mosaic events in a sample, which provide a significantly higher challenge as the mosaic level could match the fetal fraction signal. An example univariate calculation is provided below.

For a sample with observed short2long t-stat score and estimated fetal fraction [symbol] ff [symbol] _est, the likelihood ratio is:

$$LR = \frac{{\sum }_{{ff}_{total}} q ( {ff}_{total} ) * {p}_{1} ( {T}_{short 2 long} \vert {ff}_{est} )}{{p}_{0} ( {T}_{short 2 long} )} \qquad Equation A$$

where q(ffₜₒₜₐₗ) is a density distribution of fetal fraction (estimated from training data) considering the error associated with fetal fraction estimation, p₁ represents the likelihood that data come from a multivariate normal distribution representing a 3-copy or 1-copy model, p₀ represents the likelihood that data come from a multivariate normal distribution representing a 2-copy model, and T_{short2long} are T scores calculated from chromosomal coverage generated from short fragments and all fragments. In general, the short2long univariate process comprises 1) computing short/long ratio for each individual bin, then 2) running the bin ratios through bin normalization.

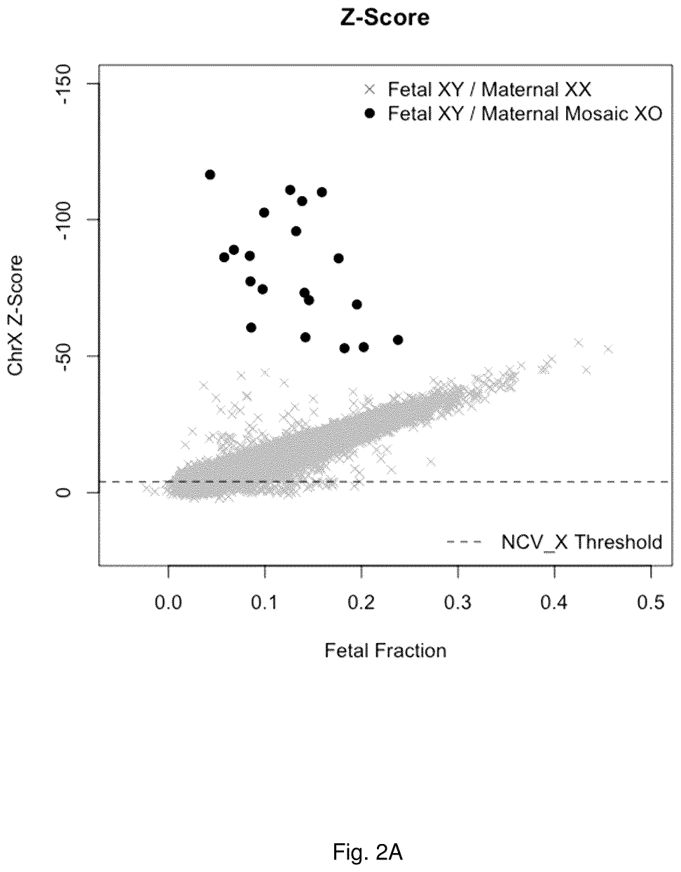

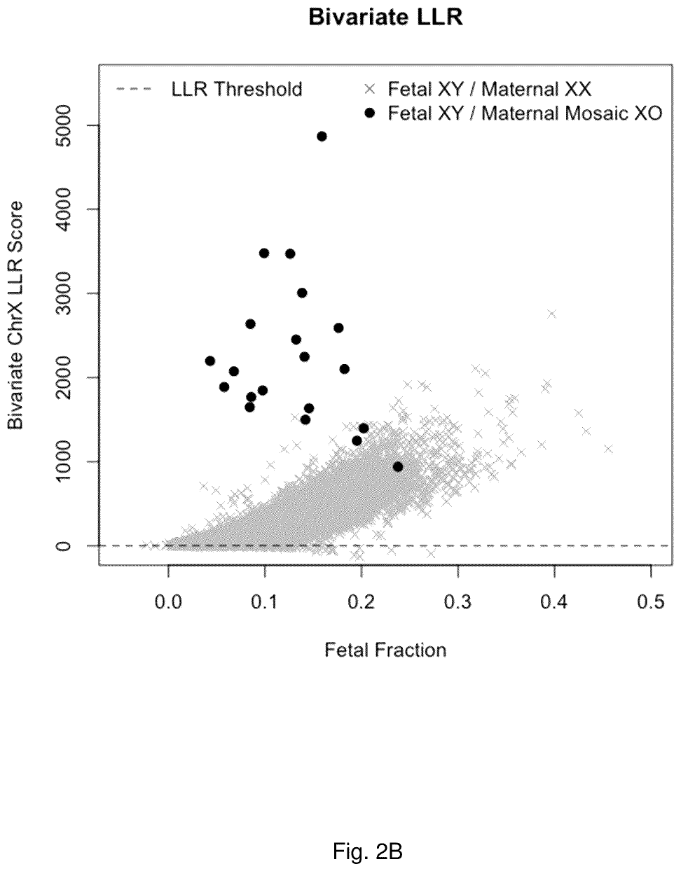

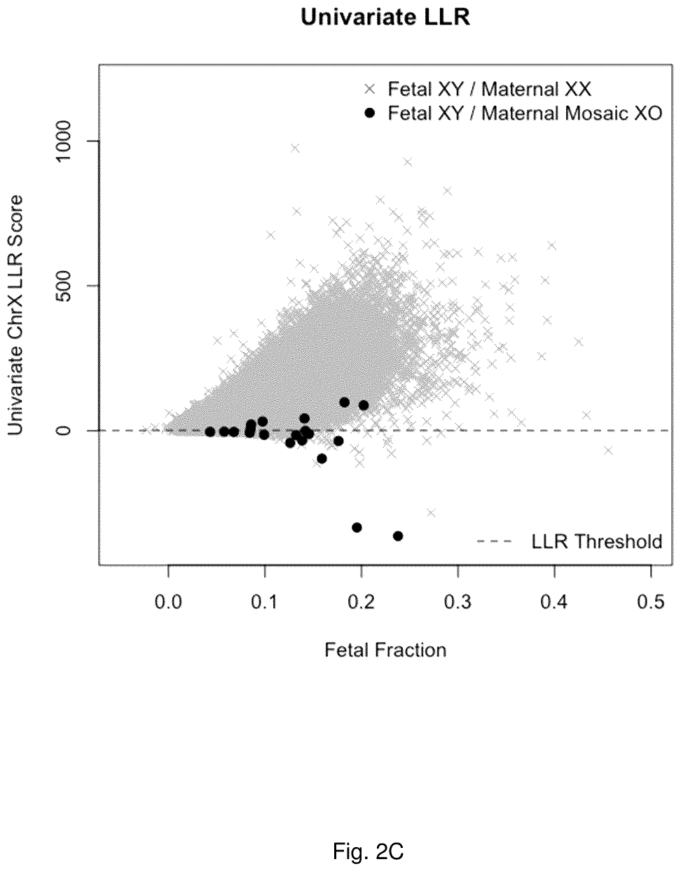

One limitation to the approach above is it does not identify instances where both the fetus and the mother contain an event in the same region. To provide an example of this, FIGS. 2A-2C summarize the same relationship as in FIGS. 1A-1C, but examining the reduction in chromosome X coverage in samples with fetal XY and maternal XX, or fetal XY and maternal mosaic XO (monosomy X). As seen in FIG. 1A, maternal mosaic events result in a z-score above the threshold (FIG. 2A), though this time both the fetus and mother are contributing to the signal. In FIG. 2B, because the event from the mother is mosaic, some of the scores trend similarly to the fetal fraction observed, suggesting the mosaic contribution from the mother is small. However, because the fetus is also contributing a signal, a subset of the univariate LLRs is still above the final threshold (FIG. 2C).

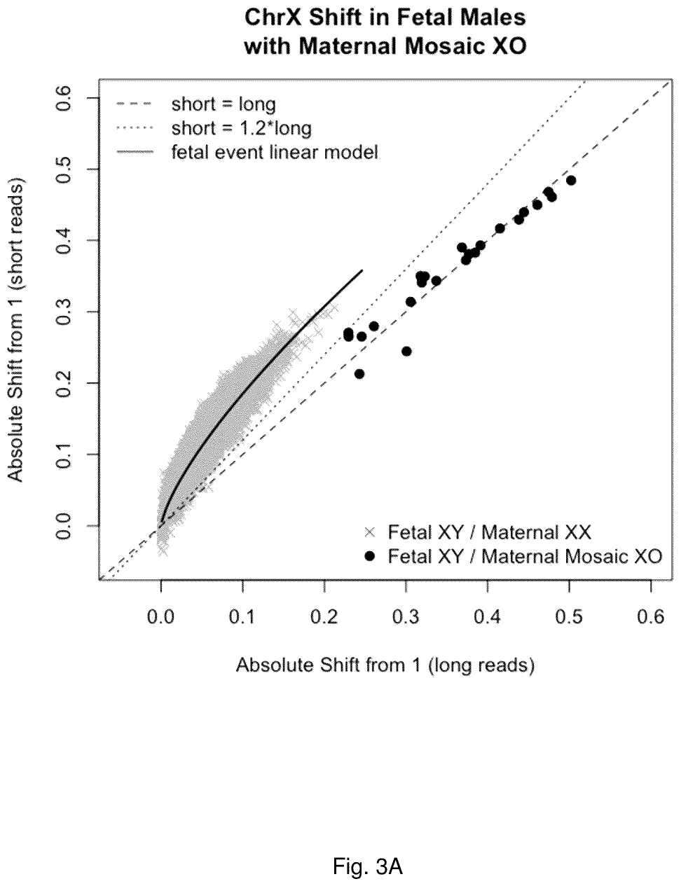

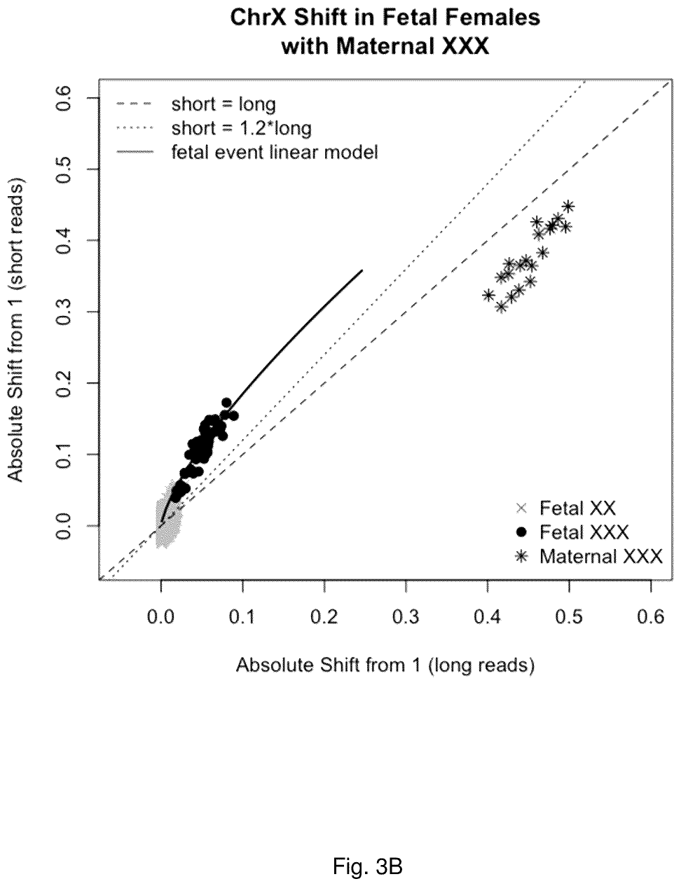

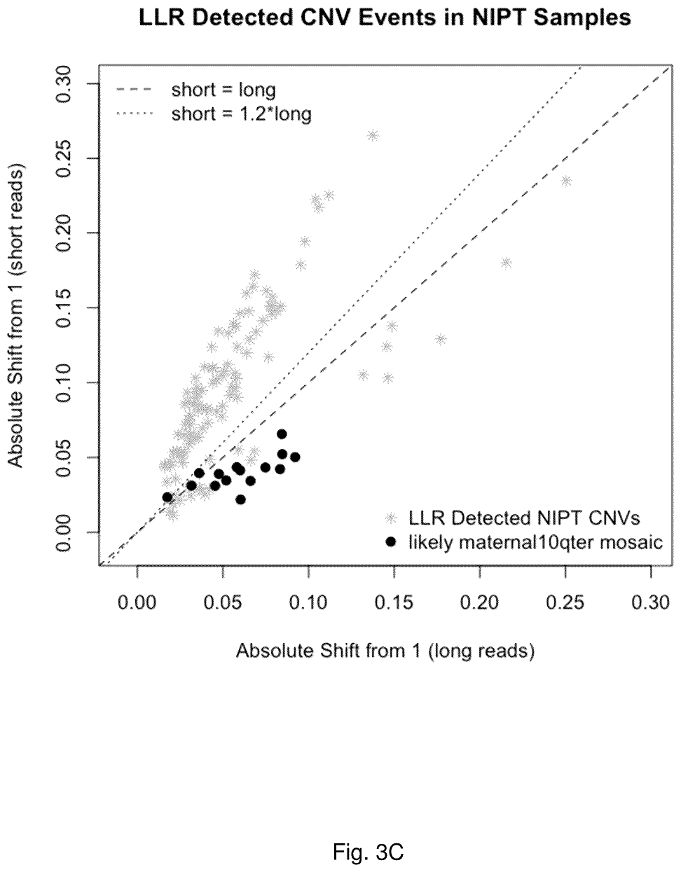

A solution is to examine the relationship between short fragment and long fragment coverage directly. In this example, short fragments are 80-150 bp in length and long fragments are 50-300 bp in length. FIG. 3A illustrates this approach, comparing the absolute shift in short and long read signals in the same dataset. Events of only fetal origin will have a noticeable enrichment in short read signal relative to long read signal, which trends with the linear model defined by the solid curve. Samples containing both maternal and fetal events in the same chromosome will result in similar levels of signal from both short and long reads, thereby trending around a 1/1 ratio (dashed line). Events only maternal in origin would have a lower signal in short reads relative to long, as illustrated in the fetal XX samples with maternal XXX events (FIG. 3B). A Variable Insert-Size Comparison QC (VIC-QC) metric computing the relative ratio can be computed for each sample, and a threshold is applied to separate events likely of maternal vs. fetal origin (threshold example depicted by the dotted line in FIGS. 3A-3C). An example VIC-QC calculation is provided below.

$$VIC\_QC = \frac{\vert 1 - {μ}_{short} \vert}{\vert 1 - {μ}_{long} \vert} \qquad Equation B$$

where μ=mean of normalized bin counts for a particular region (e.g., CNV, chromosome, etc). Fetal events will have VIC_QC˜2 (as the fetal DNA is enriched when considering short reads only relative to long reads). VIC_QC is computed from normalized short_reads and normalized long_reads, while the univariate LLR computes the ratio first on a bin level, and then normalizes those bin counts.

FIG. 3C illustrates the relationship between short and long coverage directly in the context of CNV events. NIPT samples were filtered to only include those with a single autosomal CNV event identified by existing NIPT software (the LLR was above the CNV threshold of 19, and the CNV size is >5 Mb). Each datapoint corresponds to a single sample, though events could originate from any autosomal chromosome and vary in size. Fifteen samples contained events in the 10qter region (10q23 to 10q26.3). Previous studies have suggested maternal mosaic 10qters are associated with NIPT false positive results (Huijsdens-van Amsterdam K, et al. Genet Med. 20: 1472-1476 [2018]). Here, it was observed that a majority of the samples lack short read signal enrichment and suggest these events are likely maternal in origin. Applying a ratio threshold of 1.2, these samples would be identified, reducing the number of false positives reported.

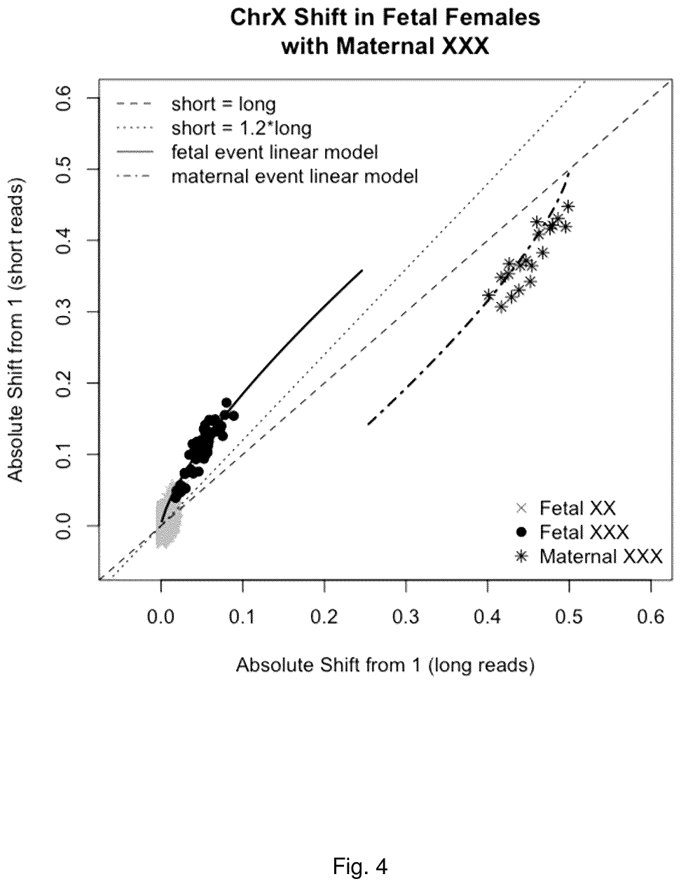

The relationship between short and long read coverage can be further leveraged to identify instances of CNV inheritance (where both the fetal and mother are impacted with the same event) and to quantify maternal mosaicism. As depicted in FIG. 3B, the relationship between short and long read coverage from a fetal event can be described by a linear model. The absolute shift from 1 in short reads corresponds to the fetal fraction estimate of the sample. If the event only originates from the mother and is not mosaic, a linear relationship is observed, which is the reverse complement of the fetal linear trend (FIG. 4). In the presence of a maternal mosaic event, the origin (the datapoint corresponding to a fetal fraction of 0%) of either the fetal or maternal linear model traverse across the identity line proportional to the level of maternal mosaicism. If the maternal event is mosaic, the level of mosaicism would dictate the origin of the linear trend on the identity line. For example, while a non-mosaic maternal XXX event origin would be located at 0.5 for short and long read shifts (as seen in FIG. 4), if the XXX event was only 50% mosaic, the origin of the maternal event linear trend would start at 0.25 instead. If an event is inherited, there should be no enrichment at all and thus samples would fall on the identify line.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. A method for determining presence, absence, or no call of a copy number variation (CNV) of a nucleic acid sequence of interest in a test sample comprising cell-free nucleic acid fragments originating from two or more genomes, the method comprising:
(a) receiving sequence reads obtained by sequencing the cell-free nucleic acid fragments in the test sample;
(b) aligning the sequence reads of the cell-free nucleic acid fragments or aligning fragments containing the sequence reads to bins of a reference genome comprising the sequence of interest, thereby providing test sequence tags, wherein the reference genome is divided into a plurality of bins;
(c) measuring fragment lengths of at least some of the cell-free nucleic acid fragments present in the test sample;
(d) for the sequence of interest:
(i) generating a first test sequence tag quantification for fragments within a first selected fragment length range; and
(ii) generating a second test sequence tag quantification for fragments within a second selected fragment length range, wherein the first selected fragment length range and the second selected fragment length range are different; and
(e) determining the presence, absence, or no call of a copy number variation in the sequence of interest according to the first test sequence tag quantification and the second test sequence tag quantification.

2. The method of claim 1, wherein the copy number variation (CNV) is a chromosome aneuploidy, a partial chromosome aneuploidy, microduplication, or microdeletion.

3. The method of claim 1, wherein the sequence of interest comprises a chromosome or part a chromosome.

4. The method of claim 1, wherein the first selected fragment length range is about 80 bases to about 150 bases.

5. The method of claim 1, wherein the second selected fragment length range is about 50 bases to about 300 bases.

6. The method claim 1, wherein:
generating the first test sequence tag quantification comprises determining a number of sequence tags aligning to each bin in the sequence of interest, wherein the sequence tags are from fragments within the first selected fragment length range; and
generating the second test sequence tag quantification comprises determining a number of sequence tags aligning to each bin in the sequence of interest, wherein the sequence tags are from fragments within the second selected fragment length range.

7. The method of claim 6, wherein:
the first test sequence tag quantification comprises a measure of central tendency for the numbers of sequence tags aligning to the bins in the sequence of interest; and
the second test sequence tag quantification comprises a measure of central tendency for the numbers of sequence tags aligning to the bins in the sequence of interest.

8. The method of claim 7, wherein the measure of central tendency is a mean.

9. The method of claim 1, wherein:
the first test sequence tag quantification comprises an absolute value of a shift from a fixed value, and
the second test sequence tag quantification comprises an absolute value of a shift from a fixed value.

10. The method of claim 1, further comprising determining a fraction of fragments originating from a first genome in the two or more genomes for the test sample.

11. The method of claim 10, wherein the fraction of fragments originating from the first genome is determined according to the first test sequence tag quantification for fragments within the first selected fragment length range.

12. The method of claim 11, wherein the fraction of fragments originating from the first genome corresponds to an absolute value of a shift of the first test sequence tag quantification from a fixed value.

13. The method of claim 1, further comprising determining whether one or both genomes in the two or more genomes for the test sample comprise the CNV.

14. The method of claim 13, wherein a determination that both genomes comprise the CNV, is based, in part, on a ratio of the first test sequence tag quantification to the second test sequence tag quantification, wherein the ratio value is about 1.

15. The method of claim 1, further comprising determining presence or absence of a mosaicism for a genome in the two or more genomes for the test sample or determining a level of a mosaicism for a genome in the two or more genomes for the test sample.

16. The method of claim 15, wherein the mosaicism is a maternal mosaicism.

17. The method of claim 16, wherein the presence, absence, and/or level of maternal mosaicism is determined, in part, according to the second test sequence tag quantification for fragments within the second selected fragment length range.

18. The method of claim 17, wherein the presence, absence, and/or level of maternal mosaicism is determined, in part, according to an absolute value of a shift of the second test sequence tag quantification from a fixed value.

19. The method of claim 1, further comprising generating a report for the presence, absence, or no call of a copy number variation in the sequence of interest determined in (e).

20. The method of claim 1, further comprising prior to (a), sequencing the cell-free nucleic acid fragments in the test sample by a non-targeted massively parallel sequencing process.
